(19) United States
(12) Patent Application Publication
(10) Pub. No.: US 2026/0094679 A1
(43) Pub. Date: Apr. 2, 2026

(54) ON DEMAND BIOSPECIMEN COLLECTION

(71) Applicant: Patricia Tawadros, Middletown, DE (US)

(72) Inventor: Patricia Tawadros, Middletown, DE (US)

(21) Appl. No.: 19/282,061

(22) Filed: Jul. 28, 2025

Related U.S. Application Data
(60) Provisional application No. 63/676,441, filed on Jul. 29, 2024.

(51) Int. Cl. G16H 10/40; G06Q 20/42

(52) U.S. Cl. CPC G16H 10/40; G06Q 20/42

(57) ABSTRACT

A computer-implemented system and method for administrating human biospecimen collection facilitates secure, de-identified coordination among donors, scientists, and administrators through a modular digital interface. The system enables administrators to manage project dashboards, assign donor identities to sample jobs, initiate and track collections, configure lab and location settings, and process payments using integrated financial services. Scientists can create, schedule, and monitor biospecimen requests, set donor inclusion criteria, and manage sample delivery. Donors can register, undergo identity verification, opt in to sample types, and track donation history. The platform supports automated notifications, real-time courier tracking, configurable appointment durations, and differential compensation models. Technical benefits include enhanced data integrity via role-based access control, improved efficiency through integrated scheduling and payment APIs, and reduced administrative burden via automated matching and logistics coordination. The system provides compliance with privacy standards while enabling scalable, institution-specific or marketplace-based biospecimen acquisition.

### CROSS-REFERENCE TO RELATED APPLICATION

This application is a U.S. Non-Provisional Utility Patent Application which claims priority co-pending U.S. Provisional Ser. No. 63/676,441 , filed on Jul. 29, 2024 entitled, contents of which are hereby fully incorporated by reference in its entirety.

### FIELD OF THE EMBODIMENTS

The present disclosure relates generally to systems and methods for managing human biospecimen procurement, and more particularly to computer-implemented platforms and interfaces configured to facilitate the secure, de-identified coordination, collection, and delivery of biospecimens from donors to researchers.

### BACKGROUND OF THE EMBODIMENTS

A biological specimen (also called a bio sample) is a biological laboratory specimen held by a biorepository for research. Biospecimens have been essential in biomarker discovery as far back as the 1980s. When biological specimens are stored, ideally, they remain equivalent to freshly-collected specimens for the purposes of research. Generally, human biological specimens are stored in a type of biorepository called a biobank. Typical types of biospecimens include, but are not limited to, cheek tissue, saliva, blood, tissue, urine, DNA, and the like. Biospecimens are extracted from human donors, at least a portion of which are selected based on their physiological profile. This profile is defined by medical information unique to the donor, which is sensitive information protected by numerous laws, regulations, and procedures.

Biospecimen procurement is the systematic process of acquiring, storing, and managing biospecimens for research for the advancement of personalized medicine and drug development. Patient-driven biospecimens are often ethically procured by public and private biobanks. These biobanks generally ensure biospecimen collection, processing, storage and shipment. Biospecimens are delivered to end user's, which include academic research institutes, government research programs, and Pharma/Biotech/Diagnostics companies. Researchers use human-derived biospecimens to develop novel drugs, therapies, and diagnostics.

Conventional procurement is dependent on the type of biospecimen needed (e.g., blood, tissues, cell products, custom-matched sets, etc.). Biological sample handling and storage procedures for biospecimen collection is typically standardized. The Standard PREanalytical Code (SPREC v2.0 and v3.0) is a coding system that reports how variations in biospecimen characteristics can impact downstream results. Many biospecimen collection and procurement processes have been fraught with difficulties, including issues with sample quality, collection, storage, and delivery, which also contribute to carbon emissions. Furthermore, the procurement process often entails substantial administrative overhead, including those related to Institutional Review Boards (IRBs), which are established to safeguard the rights, welfare, and privacy of donors. In short, conventional biospecimen procurement processes are costly, cumbersome, and fraught with difficulties. What is needed is a streamlined approach, that cuts through artificial barriers, manages biological viability concerns, and ensures donor rights and privacy are maintained.

### SUMMARY OF THE EMBODIMENTS

One aspect of the disclosure is for a system for on-demand biospecimens. Preferably, fresh biospecimens are directly acquired from employees of a company requesting the biospecimens. Other embodiments exist where donors are not necessarily employees. Because biospecimens are obtained on demand and often within a short radius of the requestor, there is no need to rely on a biobank or other intermediary storage center and there are minimal impacts on carbon emissions.

From a donor perspective (see FIG. 2C), a donor signs up to provide biospecimens through a computer, tablet, or smartphone with interacts with a server, referred to herein as the Donor X server. Signing up is part of registration, where the donor provides their consent and relevant data, where the relevant data includes general user information and user health information. The digital conveyance of relevant data occurs securely, such as over a secure communication channel requiring two factor authentication. Once a donor has successfully registered, they are assigned a unique donor identification number in order to de-identify them to researchers. A registered user establishes a profile, where the user specifies the type(s) of samples they are willing to provide and provides consent for the same. The user may be provided with one or more home kits, which include specimen bottles, containers, etc. A user can utilize a home kit to provide a requested biospecimen. Additionally, the user can be provided with a set of labs (e.g., lab diagnostic locations) or other locations where biospecimens are able to be extracted or dropped off by the user. In one embodiment, a nationwide lab company, such as any laboratory, can be a location to which a user reports, which acquires the biospecimen whether via a drop-off or through agent assisted extraction. Extraction in this context refers to a set of actions taken to obtain a biospecimen from a donor. For example, some biospecimens may require a technician/nurse take the sample from a donor.

Once registered with the Donor X server, a donor has access to a job board (FIGS. 2A and 2B). The job board indicates a set of biospecimens that are desired, requirements for the same, and payments provided. The donor may elect to provide the biospecimen(s) for a given post, which results in the associated payment being made when the job is satisfactorily completed.

From a scientist's perspective (See FIG. 2D). the scientists or group (e.g., Pharma) wanting biospecimens signs up through a computer, tablet, or smartphone with the Donor X server. The scientist completes legal paperwork and validation associated with biospecimen acquisition. Communications occur securely, such as via secure communication channels relying on two factor authentication. After successfully registering, the scientist is permitted to post new jobs for biospecimens. For a given job, the scientist may specify a sample type, donor criteria, dates that samples are needed, and acceptable pickup locations for biospecimens. Typically, a pickup location will be a lab, such as any lab location. In embodiments, a dispatch service is available to convey biospecimens from a lab to the scientist.

In some aspects, the techniques described herein relate to a system for on-demand biospecimens including. a server including at least a processor, a data store, a transceiver, and code stored on the data store, which is executable by the processor, wherein the transceiver connects the server to a network; at least one donor computing device communication linked to the server via the network; and at least one specimen requestor computing device community linked to the server via the network, wherein the server coordinates timing and delivery of biospecimens provided by a set of donors, each utilizing a corresponding one of the at least one donor computing device, to a set of biospecimen requestors, each utilizing a corresponding one of the at least one specimen requestor computing device, wherein the biospecimens are provided by the specimen donors within three days of a request from the specimen requestor without any of the biospecimens being stored in a biobank or equivalent intermediary.

In some aspects, the techniques described herein relate to a system, wherein the biospecimens as received by and requested from the specimen requestor are fresh and are never frozen between a period extracted from any of the specimen donors and received by the specimen requestor.

In some aspects, the techniques described herein relate to a system, wherein each of the specimen donors are employees of the specimen requestor, said specimen donors having privacy and medical information protected in an FDA and HIPAA compliant manner, facilitated by the server.

In some aspects, the techniques described herein relate to a system, wherein the specimens are collected and delivered in a same building, wherein specimen requestors receive the specimens within one hour of collection.

In some aspects, the techniques described herein relate to a system, wherein the specimen donors are volunteers who are paid for each specimen provided, wherein payment amounts are displayed within request messages prior to the specimen donors agreeing to provide a related biospecimen.

In some aspects, the techniques described herein relate to a computer-implemented method executed by a biospecimen procurement platform including one or more processors and memory storing instructions that, when executed, cause a system to perform operations including: receiving, via an authenticated user interface, a structured electronic request from a scientist client device, the request including one or more of a biospecimen type, collection instructions, a subject demographic, or health criteria, and a desired delivery timeframe; generating, using a scheduling engine, a biospecimen job including one or more digitally indexed appointment slots, each slot including metadata specifying a collection location, collection time, biospecimen type, and packaging specification; executing a donor eligibility determination using a matching engine configured to apply rule-based and machine-learned models to filter a donor dataset based on demographic, geographic, and physiological safety thresholds, including longitudinal blood draw volume analysis; transmitting, via a push-notification messaging module, a set of donor-specific job invitations to mobile devices of eligible donors, the invitations including appointment metadata and system-generated response options, while concealing an identity of the scientist; receiving, from at least one donor device, a secure acceptance of an appointment slot, subject to confirmation that a digitally signed consent form of the donor is current, a travel radius constraint is met, and the donor is within a safe donation threshold; reserving the accepted appointment slot and generating an appointment confirmation object including a cryptographic job token, appointment timestamp, and location metadata, which is transmitted to the donor device; prior to the appointment time, initiating a timed notification workflow that includes calendar sync, secure rescheduling tokens, and SMS/email reminders to the donor; in response to a cancellation, reinitiating a donor matching and an invitation process via an automated feedback loop until the appointment is reassigned; receiving, from a laboratory information management system or integrated collection terminal, a structured data packet indicating collection confirmation, the packet including scanned container label identifiers, job ID, timestamp, and donor ID; updating, using a backend processing module, a scientist dashboard associated with the job to reflect the collection confirmation, de-identified donor metadata, and specimen tracking status; initiating a donor payment transaction via a digitally integrated payment processor upon the confirmation of collection, wherein the transaction is linked to a verified payment method of the donor stored in encrypted form; and logging a job lifecycle including one or more of a consent, an eligibility match, an appointment assignment, a specimen collection, or a payment confirmation in a queryable system database configured for regulatory auditing and data export.

In some aspects, the techniques described herein relate to a method, wherein the donor eligibility determination includes calculating a safe blood draw volume using historical donation data stored in a time-indexed database and automatically disqualifying donors exceeding regulatory limits.

In some aspects, the techniques described herein relate to a method, wherein the cryptographic job token is a signed identifier generated using a private key stored on a hardware security module to verify integrity of the job metadata.

In some aspects, the techniques described herein relate to a method, wherein the push-notification module delivers invitations via a third-party API supporting multi-channel delivery and records delivery status and interaction metrics for adaptive donor engagement.

In some aspects, the techniques described herein relate to a method, wherein the collection confirmation includes scanning a machine-readable label affixed to each biospecimen container, the label encoding the donor ID, job ID, and timestamp using a QR or barcode format.

In some aspects, the techniques described herein relate to a method, wherein the payment processor executes transactions via an API call to a third-party platform and logs payment status in real time.

In some aspects, the techniques described herein relate to a method, further including assigning one or more backup donors to the job using a predictive model trained on historical no-show data and dynamically adjusting backup ratios based on region and sample type.

In some aspects, the techniques described herein relate to a method, wherein the scientist dashboard includes an export function configured to generate a digitally signed report file including donor health metadata and chain-of-custody timestamps.

In some aspects, the techniques described herein relate to a method, wherein the platform verifies user identity using a multi-factor authentication process that includes a biometric or knowledge-based identity check and an SMS-based one-time password.

In some aspects, the techniques described herein relate to a method, wherein the system executes a consent verification engine to confirm that the donor's digitally signed informed consent form is valid, current, and linked to the requested biospecimen type.

In some aspects, the techniques described herein relate to a computer system with a platform for coordinating biospecimen collection, including: one or more processors and a non-transitory memory storing executable instructions configured to: receive a biospecimen request from a scientist interface; create a job including appointment slots with metadata including biospecimen types, collection location, and timing constraints; execute a donor matching engine to evaluate donor eligibility using profile data, longitudinal health history, and spatial-temporal constraints; generate and transmit dynamic electronic invitations to eligible donors while maintaining de-identification of the scientist; receive donor responses and reserve accepted appointment slots; synchronize calendar and notification systems for donor reminders and rescheduling; interface with laboratory systems to receive structured collection confirmations; update a scientist-facing dashboard with collection status and de-identified donor information; initiate secure donor payment using verified financial data; and store job lifecycle data in a secure, queryable database for reporting and compliance.

In some aspects, the techniques described herein relate to a system, further including a container label generator configured to produce machine-readable specimen labels using specimen-specific and donor-specific encrypted metadata.

In some aspects, the techniques described herein relate to a system, wherein the donor matching engine applies a trained neural network model to predict donor acceptance likelihood based on historical participation, location, and compensation preferences.

In some aspects, the techniques described herein relate to a system, wherein the platform integrates with external laboratory systems using RESTful APIs and transmits real-time updates regarding appointment bookings, specimen readiness, and courier status.

In some aspects, the techniques described herein relate to a system, wherein the scientist dashboard supports one or more of real-time job filtering, longitudinal tracking of participating donors, or tagging of donors to curated study lists for future recruitment.

### BRIEF DESCRIPTION OF THE DRAWINGS

The present disclosure may be better understood, and its numerous features and advantages made apparent to those skilled in the art, by referencing the accompanying drawings. The use of the same reference symbols in different drawings indicates similar or identical items.

FIG. 1 shows an on-demand biospecimen system in accordance with embodiments of the disclosure.

FIG. 2A shows an operational sequence of the system performing donor sequence matching implemented using a GUI of a display of an electronic device such as, a donor SCREEN for a graphical user interface of a biospecimen system in accordance with embodiments of the disclosure.

FIG. 2B shows an operational sequence of the system performing donor sequence matching implemented using a GUI of a display of an electronic device such as, a donor job detail screen accordance with embodiments of the disclosure.

FIG. 2C shows an operational sequence of the system performing donor sequence matching implemented using a GUI of a display of an electronic device such as, a donor profile screen in accordance with embodiments of the disclosure.

FIG. 2D shows an operational sequence of the system performing donor sequence matching implemented using a GUI of a display of an electronic device such as, a requestor specimen screen in accordance with embodiments of the disclosure.

FIG. 3 shows a model computing device utilized with the biospecimen system in accordance with embodiments of the disclosure.

FIG. 4A is a flowchart illustrating an example computer-implemented method performed by a biospecimen procurement platform in accordance with embodiments of the disclosure.

FIG. 4B is a flowchart that continues from FIG. 4A and illustrates further steps in the biospecimen request and fulfillment workflow performed by the system, in accordance with embodiments of the disclosure.

FIG. 5 is a flowchart illustrating an example administrative workflow within the biospecimen procurement platform, in accordance with embodiments of the disclosure.

FIG. 6 illustrates an example user interface of a sample collection coordination module within the administrative platform, in accordance with embodiments of the disclosure.

FIG. 7 illustrates an example user interface of a sample collection coordination module within the administrative platform, in accordance with embodiments of the disclosure.

FIG. 8 illustrates an example user interface of a sample collection coordination module within the administrative platform, in accordance with embodiments of the disclosure.

FIG. 9 illustrates an example graphical user interface of the administrative project dashboard within the administrative platform, which provides real-time oversight of biospecimen sample requests, statuses, delivery schedules, lab and campus assignments, and associated requestors, in accordance with embodiments of the disclosure.

FIG. 10 illustrates an example graphical user interface illustrating the Donation Reports view within the administrative platform, in accordance with embodiments of the disclosure.

FIG. 11 illustrates an example graphical user interface showing the Pharma Management view within the administrative platform, in accordance with embodiments of the disclosure.

FIG. 12 illustrates an example donor profile settings interface within the donor platform, in accordance with embodiments of the disclosure.

FIG. 13 illustrates an example donor health questionnaire interface within the donor platform, in accordance with embodiments of the disclosure.

FIG. 14 illustrates an example upcoming donations interface within the donor platform, in accordance with embodiments of the disclosure.

FIG. 15 illustrates an example user interface within the donor platform for configuring email and SMS notification preferences associated with various donation-related events, in accordance with embodiments of the disclosure.

FIG. 16 illustrates an example user interface within the donor platform, depicting the Get Paid settings screen configured for a donor to select and configure a preferred method for receiving compensation for completed donations, in accordance with embodiments of the disclosure.

FIG. 17 illustrates an example user interface within the donor platform displaying job-specific donation details, in accordance with embodiments of the disclosure.

FIG. 18 illustrates an example user interface within the scientist platform displaying the sample request interface, in accordance with embodiments of the disclosure.

FIG. 19 illustrates an example user interface within the scientist platform showing requestor identity fields, project details, and donor inclusion/exclusion criteria for generating a new biospecimen request, in accordance with embodiments of the disclosure.

FIG. 20 illustrates an example user interface within the scientist platform allowing a scientist to configure matched or unmatched sample requirements and specify donor recruitment criteria by donor ID or demographic attributes, in accordance with embodiments of the disclosure.

FIG. 21 illustrates an example user interface within the scientist platform that enables a scientist to configure exclusion criteria for donors and define detailed blood and urine sample collection parameters, in accordance with embodiments of the disclosure.

FIG. 22 illustrates example user interface within the scientist platform depicting the “Parent Studies” module, which allows scientists to organize and manage long-term or multi-phase biospecimen collection projects by grouping related sample requests under a shared study header, in accordance with embodiments of the disclosure.

FIG. 23 illustrates an example user interface within the scientist platform enabling requestors or scientists to update personal, departmental, and sample delivery information for accurate system communication and biospecimen dispatch, in accordance with embodiments of the disclosure.

FIG. 24 illustrates an example user interface within the admin platform depicting the section for setting appointment durations and compensation values for various biospecimen types, in accordance with embodiments of the disclosure.

### DESCRIPTION OF THE PREFERRED EMBODIMENTS

The preferred embodiments of the present invention will now be described with reference to the drawings. Identical elements in the various figures may be identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

As used herein, the singular forms “a,” “an,” and “the,” are intended to include the plural forms as well, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Conventionally (see Background), biobanks are intermediaries that gather, receive, and store biological samples, which are thereafter requested by various requestors. Effectively, biobanks are the clearing house believed to be essential for compliance, security, anonymity, etc. within the process.

The system described herein supports two primary deployment configurations: an on-campus configuration and a marketplace configuration, each designed to facilitate the acquisition and coordination of biospecimen donations for research purposes. In the on-campus configuration, both the donor and the scientist (also referred to as the requestor) are affiliated with the same organization, such as a university, medical center, or research institute. This deployment mode typically leverages internal infrastructure, including institutional collection sites, administrative oversight by internal staff, and organization-approved compensation mechanisms. By contrast, the marketplace configuration enables external scientists, typically affiliated with research institutions, pharmaceutical companies, or biotechnology firms, to place biospecimen requests fulfilled by members of the general public who serve as donors. In this configuration, donors interact with third-party collection sites, mobile phlebotomy services, or direct-to-home testing kits, and compensation is generally issued via modern digital payment systems.

Both deployment modes share a unified technical infrastructure that includes common components such as identity verification tools, donor and scientist dashboards, real-time matching and scheduling algorithms, logistics coordination engines, and notification systems. Shared functions include profile management, eligibility filtering. sample-type selection, informed consent workflows, and administrative dashboards for viewing job statuses and donor activities. Additionally, a multi-stage algorithm governs eligibility filtering, donor-job scoring, appointment optimization, label generation, and logistics coordination across both systems. The algorithm incorporates real-time status updates and fallback logic to ensure continuity in the event of cancellations or no-shows, which are proactively mitigated through an overbooking strategy informed by historical platform data.

Despite the shared core, each mode includes distinctive features tailored to its use context. In the marketplace configuration, donors are sourced from the general public and typically visit national laboratories, imaging centers, or other commercial sites for sample collection. In some cases, self-test kits may be mailed to donors'homes, or mobile phlebotomists may collect specimens in person. Sample logistics, including delivery and tracking, are handled automatically through API-based courier integrations. Marketplace donors are compensated via direct deposit, peer-to-peer payment applications, or other digitally-enabled financial tools. In contrast, the on-campus configuration typically limits donors to internal staff, students, or affiliates. Sample collection may occur at designated campus labs or research units, and compensation may be administered through points-based internal reward systems or institutional payroll. Administrative users in the on-campus configuration may manage donation workflows manually, including confirming collections, entering punctuality notes, and determining when to notify scientists of sample readiness.

This bifurcated system architecture allows for flexible deployment across diverse research environments, offering tailored workflows while leveraging a scalable and cohesive technical foundation. The dual-mode platform not only enables efficient biospecimen matching and fulfillment but also provides practical technical improvements in scheduling, real-time logistics, and participant engagement across both institutional and public-facing scenarios.

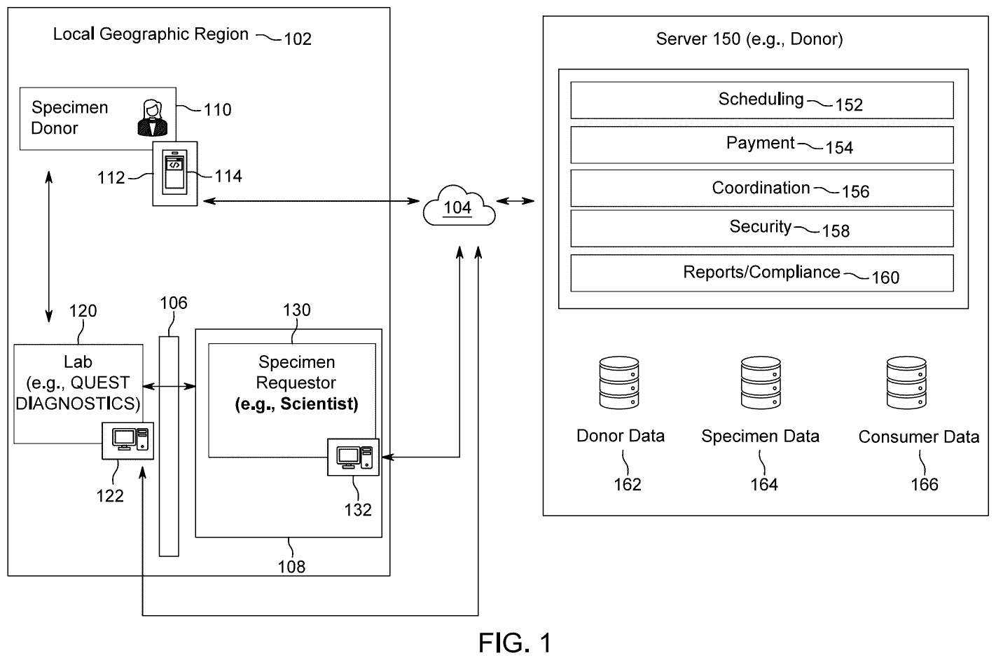

FIG. 1 shows a system for requesting, obtaining, and utilizing biospecimens in accordance with an embodiment of the disclosure. As shown, biospecimens (or bio samples) are obtained on-demand without requiring conventional intermediate storage and/or delivery, which significantly adds to the overall cost/overhead. System reimagines this process in a manner not previously believed viable/possible. That is, specimen donors 110 register with server 150 (e.g., Donor X server), which retains donor data 162. This data 162 can include Health Insurance Portability and Accountability Act (HIPAA) sensitive patient medical data. That is, biospecimens are needed for specific studies and conditions, which require extensive knowledge about a donor's (110) health. Similarly, specimen data 164 is often correlated to lab tests conducted by scientists (requestors 130). The specimens are heavily tracked in processes often overseen by the Food and Drug Administration (FDA). Server 150 is able to ethically gather data, ensure anonymity, ensure a revenue stream for the various donors 110 (via payment module 154), while minimizing overall costs and time. In one embodiment, each donor 110 and requestor 130 is an employee working for the same company and/or working within a single building. The proximity (102) ensures samples/specimens are easy to gather fresh. In a further embodiment, the devices 112, 132, 136 can be company owned ones used by employees (110 and 130), which have been extended/adapted to permit volunteers to become specimen donors 110. Firewalls 106 are established for information privacy. In embodiments, the server 150 is operated by a third party to ensure the company having employees (110, 130) in various donor-related roles are not negatively affected by the innovations presented herein and to ensure compliance with HIPAA and the FDA.

Server 150 is the intermediary that communicates with all stakeholders, retains confidentiality of information, and ensures an information/identity firewall 106 is appropriately established and maintained. Server 150 handles intake, interfaces, scheduling 152, payment 154, coordination 156, security 158, compliance and reports 160. Donor data 162, specimen data 164, and requestor data 156 are maintained by server 150. Bach stakeholder utilizes their own computing device 112, 122, 132 to communicate with server 150 via network 104.

Security module 158, which includes data segregation, anonymization, isolation, encryption, and other such features, ensures donor 110 privacy is protected, even from the underlying company for which the donor's work, and even in circumstances where the requestor 130 works in the same building as the donor 110.

The concept of establishing a ‘job’ for a biospecimen is handled by the coordination module 156 of the server 150. That is, a new “job” for a needed specimen is established, the server 150 matches a potential donor 110 for that job, the donor is scheduled (152) for a lab 120 (and/or provided a home kit and specimen drop off location). The requestor 130 is appraised (alerted by coordination module 156) of when the specimen is available for pickup. The requestor 130 may pick up the specimen at its location (e.g., lab 120) or may utilize a delivery/transport service. A set of drop off/pick-up locations (e.g., lab 120) such as those of QUEST DIAGNOSTICS can be established to ensure anonymity is maintained, in one embodiment.

In some embodiments, a set of potential donors 110 can register with server 150, indicating that each is willing to provide biospecimens in the future. There can be an intake/registration process, through which all relevant donor 110 data 162 is obtained. Because many specimens have very specific requirements, the donor data 162 can be quite extensive.

The donors 110 necessary resides within a proximal region, referred to as local geographic region 102, to the specimen requestor 130. In one embodiment, the donors 110 and requestor 130 may be employees of the same company. The donors 110 may be include a set of workers residing within the same building and/or building complex as the requestor 130. Donors 110 may work for or be located near the lab 120 as well. Alternatively, a set of donors 110 could periodically visit region 102 (whether they work there, or simply frequent for some reason). For example, region 102 could include a grocery/retail store (possibly with a pharmacy able to extract/provide kits for biospecimens and therefore function as lab 120), which is proximate to the requestor 130, which enables donors 110 (including shoppers) to provide biospecimens. In still another embodiment, the donors 110 can be students attending a research university, which is the requestor 130. The research university may have a student health clinic, which functions as lab 120. In another embodiment, the sample request is not available from a local donor. The donor may self-collect a sample using a home test-kit and take it to a delivery partner with a QR code to be scanned, packed, and shipped to the requestor while maintaining anonymity of both the donor and the requestor; or the donor visits a local lab to have the sample collected and the lab packages the sample and ships or couriers the sample to the requestor.

In one embodiment, the system can include a server 150 (e.g., the system server), a set of donor computing devices 112, a set of requestor computing devices 132, and a set devices 122 of a lab 120 (e.g., one or more laboratories or different entity/company responsible for specimen collection and/or extraction). The various computing devices 112, 122, 132, 150 are used to coordinate the disclosed process.

The server 150 may include at least a processor, a data store, a transceiver, and code stored on the data store, which is executable by the processor. The transceiver (e.g., WIFI, BLUETOOTH, etc.) connects server 150 to a network 104. The donor 110 and requestor 130 computing devices 112, 132 are communicatively linked to the server 150 via the network 104 as are the lab 120 (e.g., specimen collection/extraction) computing devices 122. The server 150 coordinates timing and delivery of biospecimens provided by a set of donors at a specimen collection location 120 to a set of biospecimen requestors 130. The biospecimens are provided by the specimen donors without any of the biospecimens being stored in a biobank or equivalent intermediary.

Another aspect of the disclosure includes a method for obtaining biospecimens that includes enrolling a set of potential specimen donors in a program where biospecimens are requested when needed by a specimen requestor. A request is received by the server from the specimen requestor. The request is converted into a project using the system algorithm. The project includes a set of jobs; each job being satisfied by a unique one of the potential specimen donors providing a biospecimen. Responsive to establishing the project, messages are conveyed to devices of the potential specimen donors offering money for providing biospecimens responsive to one of the jobs. Donors log into the Donor X server and view the job request details on their personal job board that displays only jobs they are qualified to participate in based on their profile data. The jobs are completed by receiving biospecimens from a subset of the potential specimen donors. Each of the donors being paid an amount consistent with the offering. The specimen requestor receives the biospecimens of the set of jobs. Each biospecimen of the job is conveyed fresh to the specimen requestor within hours of being obtained if collected locally or shipped overnight. When specimens are prescreened or processed, additional time will be required for these steps.

In some embodiments, the system employs a computer-implemented, multi-stage matching and logistics optimization algorithm executed by one or more processors within a secure computing environment. The algorithm begins by accessing a structured dataset stored in system memory that includes user-submitted and verified donor profile data, such as biological sex, age range, geographic location, sample-type preferences, travel tolerances, and medical history inputs gathered via interactive graphical user interfaces. Upon receipt of a biospecimen request via the scientist dashboard, the processor executes a filtering subroutine to compare the request parameters with donor attributes using indexed queries and constraint logic. The filtering subroutine produces a reduced donor pool comprising only those who meet all specified constraints.

Next, a scoring engine applies a donor-job match scoring function, which assigns weighted scores based on multiple technical parameters such as geohash proximity, temporal slot alignment with appointment windows, longitudinal medical data flags, sample-type matching, and past participation reliability metrics stored in a local cache or distributed database. These scores are calculated using configurable weighting vectors and may include matrix operations or ranking algorithms that produce an ordered list of candidate donors. This matching algorithm is configured to run within a defined execution time threshold to support real-time responsiveness in dynamic scheduling scenarios.

A scheduling engine then interacts with external system APIs via secure REST protocols or internal scheduling modules to reserve and confirm appointment times. The scheduler dynamically evaluates system-wide variables such as current load on collection sites, courier cutoff times, and lab processing queues to optimize logistics and minimize delays. Once appointments are confirmed, the system generates encoded labels containing anonymized donor IDs, collection timestamps, sample type indicators, and job identifiers, and stores this metadata within a tamper-evident log to ensure traceability and integrity.

Further, the algorithm includes a fault-tolerant backfilling module that is triggered upon detection of no-show or cancellation events. This module uses a rolling backup queue stored in memory, selected from high-scoring alternate donors, to reassign appointments in near real time without user intervention. This algorithm will increase the compensation to align with the urgency. In certain embodiments, the system incorporates feedback loops where actual outcome data (e.g., punctuality, donor eligibility compliance, courier pickup success) is stored and used to update internal donor reliability coefficients. These coefficients are fed into a learning model, such as a decision tree or a reinforcement learning agent, that adjusts future match scoring weights to improve predictive accuracy and operational performance.

By integrating data acquisition, constraint evaluation, intelligent ranking, schedule optimization, and adaptive feedback in a unified computing framework, the system enhances the operation of computing technology itself. Unlike mere mental processes or manual matching, the described algorithm executes in a distributed, automated, and time-sensitive environment, offering technical improvements in reliability, scalability, efficiency, and traceable data governance. As such, the invention represents significantly more than an abstract idea and is directed to a practical application that improves computer functionality and the technological field of biospecimen logistics and donor-scientist coordination.

In some embodiments, the system employs a multi-stage algorithmic engine configured to manage the complete biospecimen request and fulfillment lifecycle. This biospecimen matching and fulfillment engine includes six interlinked phases: eligibility filtering, donor-job scoring, appointment optimization, logistics coordination, real-time status handling, and adaptive learning.

The process begins with eligibility filtering and donor segmentation. The algorithm retrieves structured donor data, including demographic attributes, self-reported medical history, sample-type preferences, and geographic constraints. Boolean filters are applied to eliminate ineligible candidates based on parameters such as disease state (e.g., diabetes), travel radius (e.g., 15 miles), or opt-in sample types (e.g., blood only). These filters are dynamically reconfigurable by an authorized user, enabling the platform to support tailored inclusion and exclusion criteria for each biospecimen request.

Following this, eligible donors are scored against open requests using a configurable matching algorithm. The match score is computed using a weighted sum of key parameters: location proximity (L), availability alignment (A), health history compatibility (H), and a donor reliability index (P), which reflects factors such as punctuality and historical no-show rates. The formula takes the form M=(W₁×L)+(W₂×A)+(W₃×H)+(W₄×P), where W₁-W₄ are adjustable weights set through the system's administrative interface to fine-tune prioritization logic.

Upon establishing donor-job matches, the scheduling component of the algorithm identifies viable appointment slots by interfacing with third-party laboratory APIs or internal scheduling tools. Scheduling logic accounts for constraints including required sample processing times, throughput limitations of the collection site, donor availability windows, and courier cut-off deadlines. High-scoring matches are scheduled first, and a priority queue is generated in accordance with sample urgency or delivery deadlines.

Once appointments are confirmed, the system compiles necessary metadata for logistics and label coordination. The algorithm packages de-identified donor ID, job number, collection address, sample type, and tube specifications into structured data objects for label generation. When courier integration is available, delivery tasks are automatically initiated via secure APIs, with tracking information synchronized back to the platform. The algorithm continuously monitors real-time job statuses and incorporates failover logic. If a donor cancels or fails to attend their appointment, the system checks a pre-allocated backup donor pool, often set using a 2:1 redundancy model, and issues fallback alerts and updated courier routing instructions accordingly.

Lastly, a learning and optimization layer evaluates key performance indicators over time, including match success rates, donor compliance, average time-to-collection, and delivery efficiency. Using reinforcement learning or heuristic feedback methods, the system dynamically refines scoring weights, improves predictive models for donor reliability, and optimizes end-to-end routing performance. Collectively, these phases constitute a technical improvement to the biospecimen collection process, transforming what would otherwise be a manually coordinated activity into a scalable, automated, and adaptive software solution.

An aspect of the disclosure is for a method of obtaining biospecimens that includes receiving a request for a set of biospecimens. The request specifies unique characteristics of a set of people (e.g., donors) from which biospecimens are to be obtained. The unique characteristics are matched to corresponding ones of a set of enrolled donors, which are referred to as a matching donor subset. Electronic messages are conveyed to the matching donor subset via a network. The messages can require at least one biospecimen from each donor. Ideally, each donor in the subset is geographically located within a specified radius of a collection site (e.g., any lab 120) proximate to the biospecimen requestor 130 (e.g., scientist). In the event the biospecimen is unavailable within the specified radius, the job will be collected and shipped from a donor.

An aspect of the disclosure is for a method of obtaining biospecimens that includes a direct-to-donor marketplace. Registered donors have profiles on the Donor X server that are searchable by specimen requestors. Donors will have the capability to set their price for donation according to specimen type with standard pricing for healthy donors and customized pricing possible for disease states. Specimen Requestors will use filtering tools such as but not limited to gender, age range, race, disease states, medications, etc. Once the specimen requestor finds a subset of de-identified donors that fit their needs, that subset can be saved to a set of favorites/boards (example: anemia study). These subsets can be invited by the specimen requestor to donate samples for a specific request. Electronic messages are conveyed to the matching donor subset via a network. The messages can require at least one biospecimen from each donor. Ideally, each donor in the subset is geographically located within a specified radius of a collection site (e.g., one or more labs 120) proximate to the biospecimen requestor 130 (e.g., scientist). In the event the biospecimen is unavailable within the specified radius, the job will be collected and shipped from a donor.

One aspect of the disclosure is directed to an on-demand biospecimen collection method and system, which manages an Internal Donor program where employees of a pharmaceutical company/research location/CRO/ETC are incentivized financially to provide samples to their employer, while safeguards are instituted to maintain Health Insurance Portability and Accountability Act (HIPAA) compliance and the employees are de-identified. The internal program enabled by the platform ensures the highest quality, fresh samples often available within minutes of collection, which are superior to frozen samples obtained from bio banks.

Scientists benefit from the platform, which allows scientists to submit complex and donor-matched biospecimen requests in moments. Scientists have access to fresh samples within minutes of collection. Samples are received quickly with the highest possible viability. Donors often can be recalled easily, Employees utilize the platform as they are paid for biospecimen sample donations. Employees receive notifications, such as through SMS/email alerts of available jobs. Scheduling and reminders are automated. The platform and its interactions remain fully anonymous to requests through use of a unique donor ID. Donor health is protected by built in safeguards that prevents participants from exceeding donation limitations.

Program administrators benefit from automated donor scheduling that flexes to a typical workday. Automated appointment reminders are conveyed to donors, which maintains efficiency. Request and donor anonymity is maintained, where full identities are only visible to administrators. Records for donor payments and FDA compliance are automatically handled via exportable reports. Sample requests are made downloadable to insert into an electronic lab notebook (ELN). From a pharma perspective, FDA compliance is enabled for the use of human biospecimens. The platform speeds sample acquisition from weeks to days, enabling faster “go/no-go” decisions. Sample acquisition costs are reduced by up to 75% over conventional processes. Lab productivity is increased. Overall, a sustainable ecosystem for biospecimen sample acquisition is created by the platform.

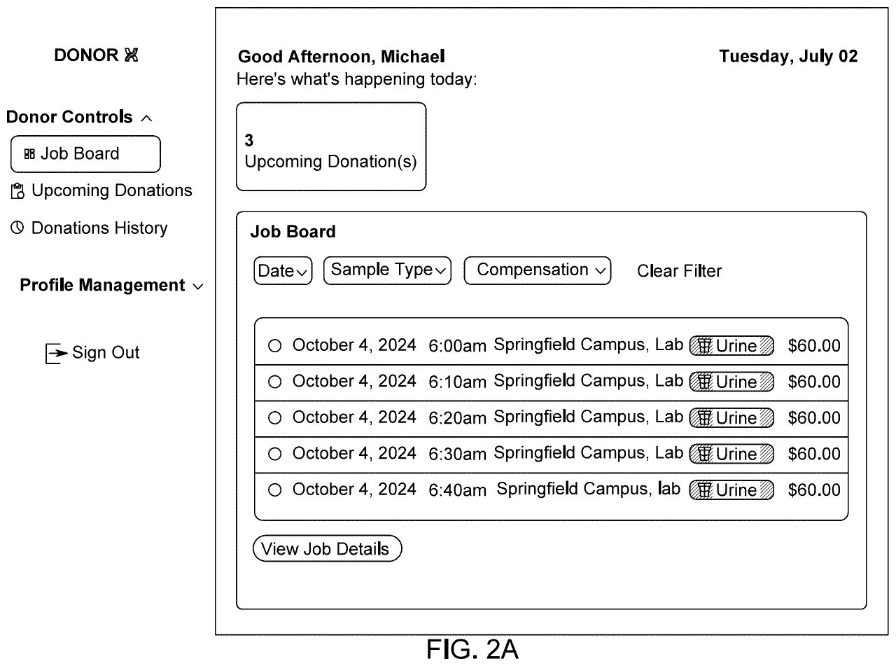

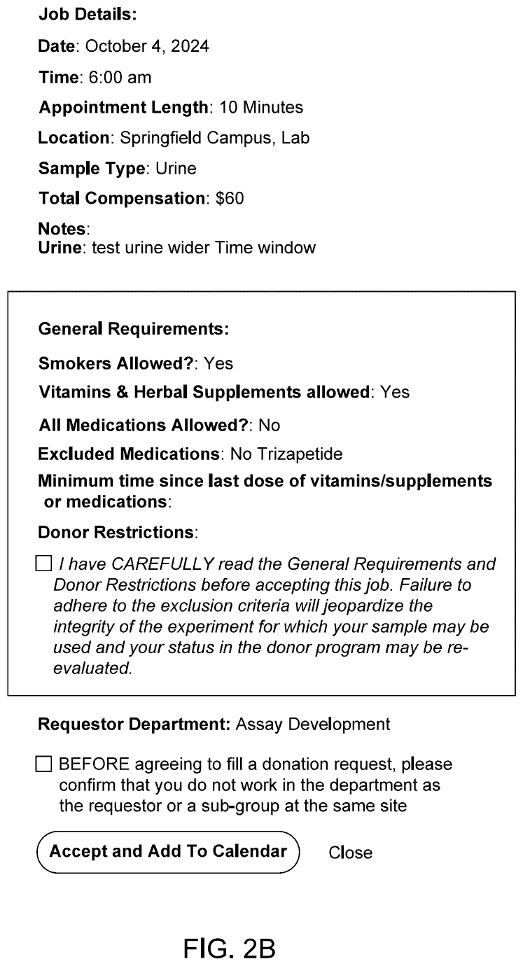

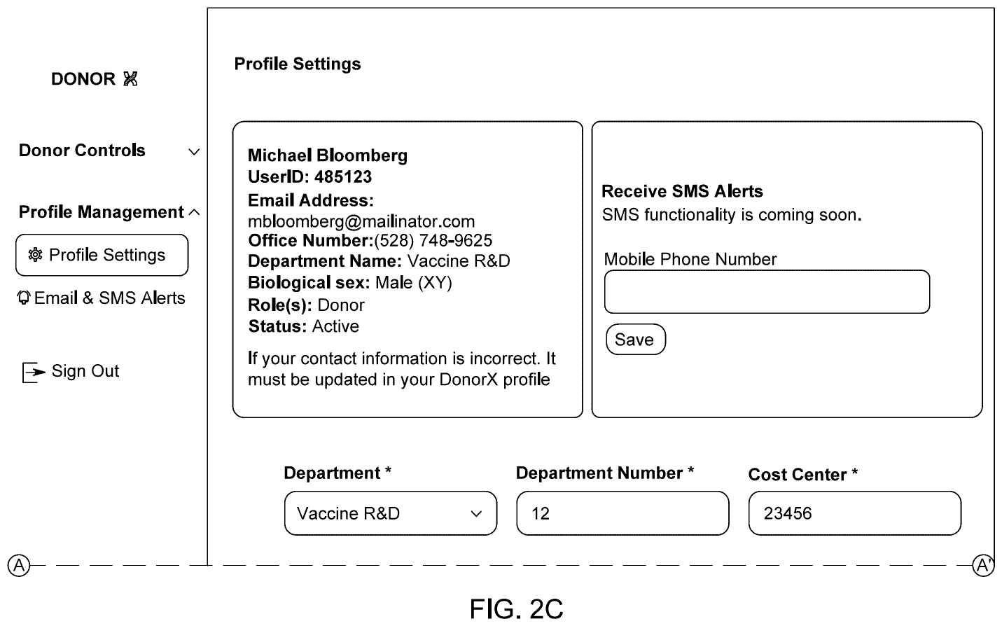

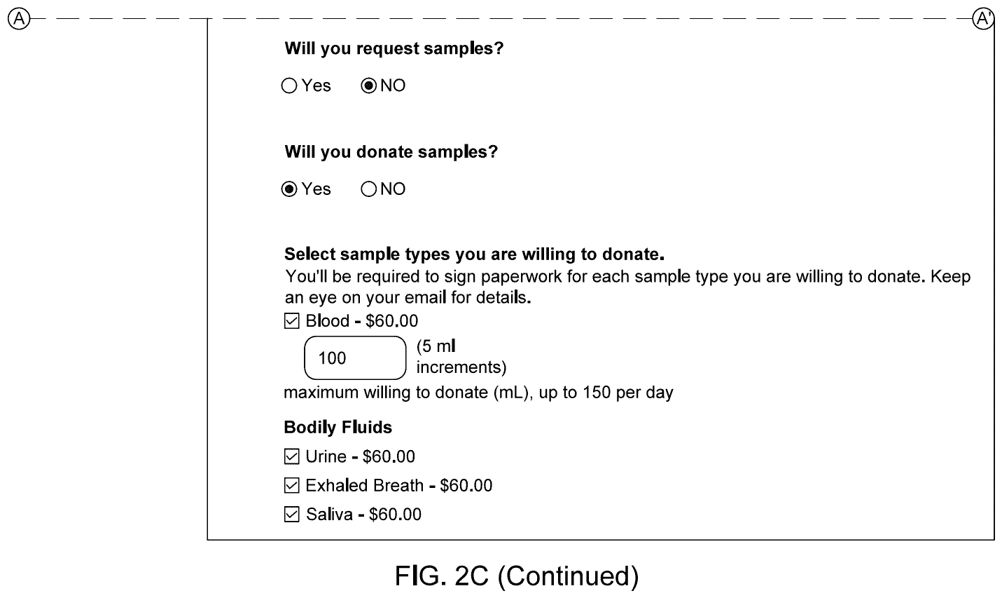

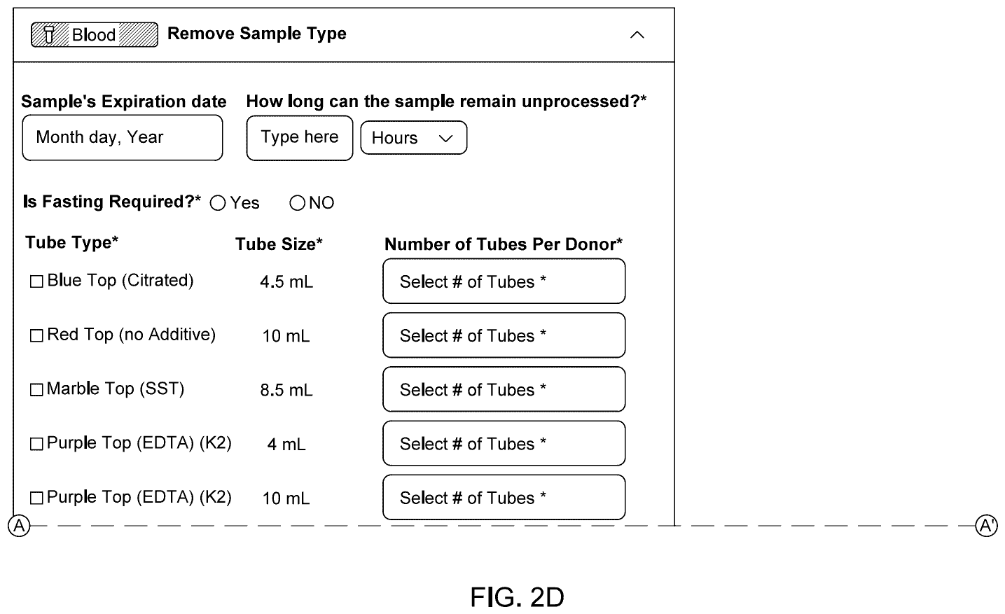

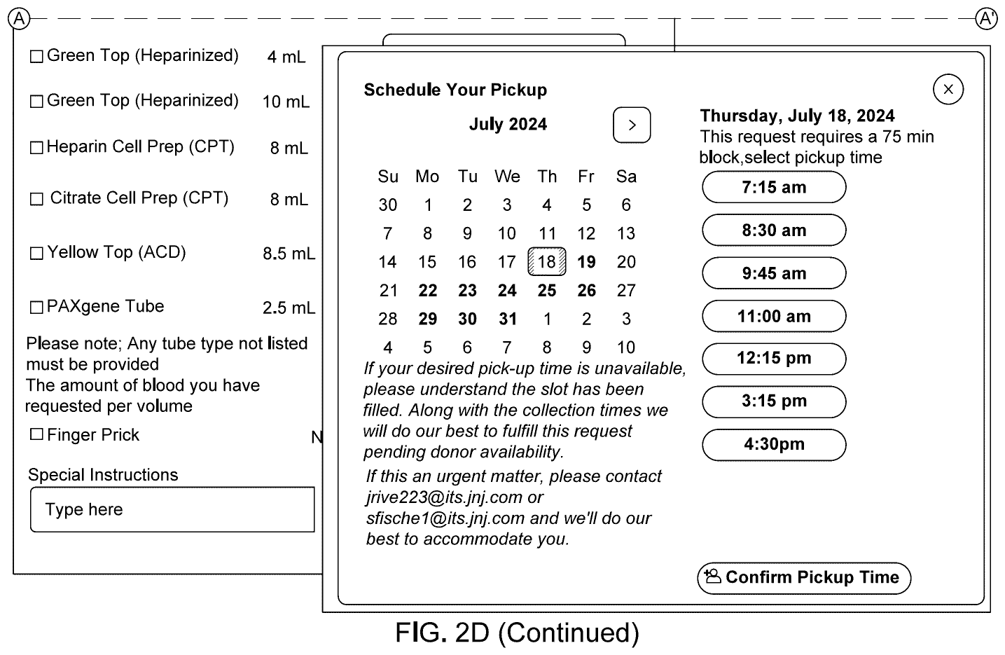

FIG. 2A shows an operational sequence 200 of the system performing donor sequence matching implemented using a GUI 202 of a display 204 of an electronic device 206 such as, a possible screen of a donor's device. As can be seen, controls exist for a job board, for upcoming donations (a calendar), and for a donation history. Management and setting screens are also included. As a screen is personalized for a specific donor, a login/logout option can be present. As shown, a daily reminder of commitments is shown such as three donations being needed. Each may have a date, time, drop off location, type, and amount associated. For example, a first appointment may exist for Oct. 4, 2024, at 6:00 Am, at the Springfield Campus Lab for a urine sample (biospecimen), for a compensation of sixty dollars.

FIG. 2B shows an operational sequence 210 of the system performing donor sequence matching implemented using a GUI 202 of a display 204 of an electronic device 206 such as, a job detail screen. This screen can show a date, time, appointment length, location, sample type, compensation, notes, and other information. The detail screen can list general requirements for participating (providing the biospecimen), as well as a confirmation button/option. For example, as general requirements can be for smokers taking vitamin and herbal supplements, but not taking TRIZAPETIDE. Additional restrictions may exist, such as not working within the department as a requestor or sub-group conducting the study. In one embodiment, an option to accept and add an appointment to a calendar is presented.

FIG. 2C shows an operational sequence 220 of the system performing donor sequence matching implemented using a GUI 202 of a display 204 of an electronic device 206 such as, a donor profile screen. Each donor can have a user ID, contact email, phone number, and other relevant information. SMS and other text alerts can be enabled or disabled. People with a profile can have distinct roles, such as being requestors 130 and donors 110.

Different types of samples can be satisfied by the system and a donor 110 can specify which types of samples he/she is willing to provide. In embodiments, maximum donation quantities of biospecimens can be established, as shown.

FIG. 2D shows an operational sequence 230 of the system performing donor sequence matching implemented using a GUI 202 of a display 204 of an electronic device 206 such as, sample information 164 available through a screen (GUD) of requestor 130. A requestor 130 can use a screen like FIG. 2D to specify a biospecimen. As shown, numerous containers (blue top 4.5 mL, redtop 10 ml) can be designated for different categories. Any container other than those listed may need to be provided specially. Any donor 110 participating receives the noted type of container for the biospecimen. Specimen viability limits and times can be specified, as can pick-up dates/times. These can be used to help coordinate scheduled lab actions, with sample/specimen acquisition.

### Systems, Devices, and Operating Environments

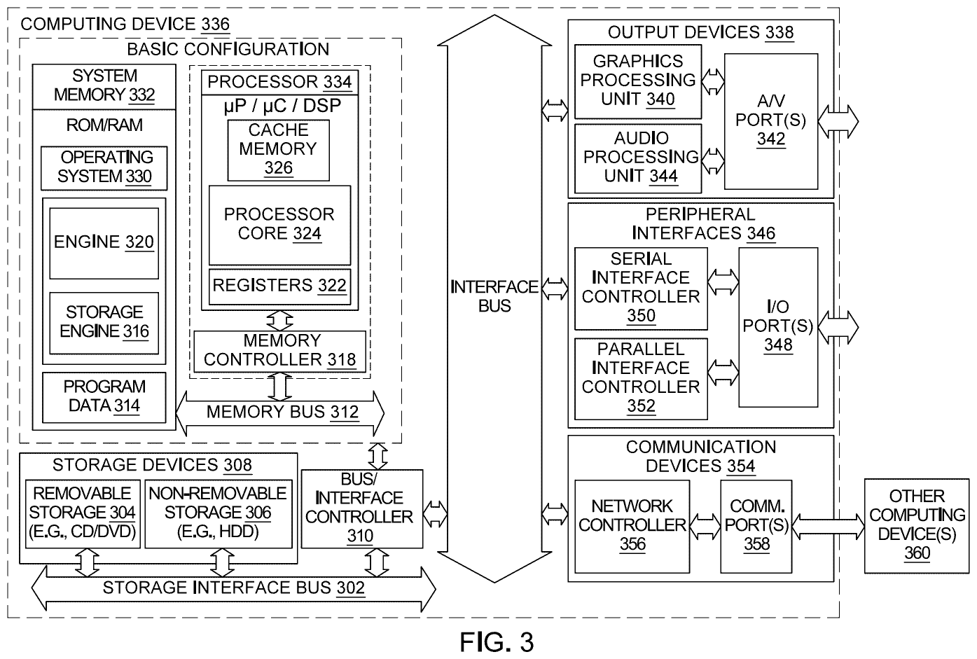

A basic configuration of a computing device is illustrated in FIG. 3, represented by the components enclosed within the inner dashed line. In this configuration, the computing device 336 includes at least one processor 334 and system memory 332. The term “processor” refers to any suitable computing unit capable of executing instructions, including, but not limited to, a microprocessor, microcontroller, digital signal processor (DSP), or any suitable combination thereof. A memory bus 312 facilitates communication between the processor(s) 334 and the system memory 332.

Referring again to FIG. 3, the processor 334 may include one or more levels of on-die memory such as cache memory 326, as well as a processor core 324 and one or more internal registers 322. The processor core 324 may include an arithmetic logic unit (ALU), a floating point unit (FPU), and optionally a digital signal processing core, among other subcomponents. In some embodiments, the memory controller 318 may be integrated with the processor, or it may be implemented as a discrete unit for interfacing with system memory.

The system memory 332 may comprise any type of volatile or non-volatile memory, including random-access memory (RAM), read-only memory (ROM), flash memory, or combinations thereof. The system memory 332 stores an operating system 330, one or more executable engines 320, and program data 314. The engine 320 may include application software, background services, data processing modules, or specialized software platforms. A dedicated storage engine 316 may be configured to manage and store data structures used by the platform described herein.

The operating system 330 may be a general-purpose or embedded system capable of multitasking, memory management, process isolation, and secure user access. Suitable operating environments may include Unix-like systems, POSIX-compliant platforms, real-time operating systems (RTOS), or other computing frameworks tailored for mobile, desktop, or server deployments. Operating system features may include modular kernel architecture, secure networking, and device driver abstraction layers, enabling efficient operation across diverse computing environments. A graphical user interface (GUI) may be used to render platform functionality to users. The GUI may include desktop environments, windowing systems, or web-based front-ends using markup languages, scripting languages, and dynamic rendering libraries. User interaction may be supported via conventional GUI frameworks or through web-based technologies such as extensible markup languages, asynchronous scripting, and modular interface toolkits. In certain configurations, the computing device may include a web browser component to retrieve and display content over a network. The browser may support secure web protocols such as HTTPS and employ encryption standards equal to or greater than 128-bit. The browser may interpret and execute client-side technologies such as script files, embedded objects, and APIs, enabling interactive features. In mobile or embedded deployments, lightweight browser engines may be used to balance responsiveness and memory efficiency.

The described configuration supports role-based interaction across distributed systems, enabling end users, whether donors, scientists, or administrators, to securely access platform functions from desktop or mobile environments. Technical benefits of this architecture include extensibility across device classes, isolation of critical execution functions, and compatibility with heterogeneous computing infrastructures, enabling reliable execution of the biospecimen collection and management workflows described in this disclosure.

A web browser may communicate to and/or with other components in a component collection, including itself, and/or facilities of the like. Most frequently, the web browser communicates with information servers, operating systems, integrated program components (e.g., plug-ins), and/or the like; e.g., it may contain, communicate, generate, obtain, and/or provide program component, system, user, and/or data communications, requests, and/or responses. Of course, in place of a web browser and an information server, a combined application may be developed to perform similar functions of both. The combined application would similarly affect the obtaining and the provision of information to users, user agents, and/or the like from the enabled nodes of the present invention.

Moreover, the computing device 336 may have additional features or functionality, and additional interfaces to facilitate communications between the basic configuration and any desired devices and interfaces. For example, a bus/interface controller is used to facilitate communications between the basic configuration and data storage devices via a storage interface bus 302. The data storage devices may be one or more removable storage devices, one or more non-removable storage devices, or a combination thereof. Examples of the one or more removable storage devices and the one or more non-removable storage devices include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus facilitates communication from various interface devices (e.g., one or more output devices 338, one or more peripheral interfaces 346, and one or more communication devices 354) to the basic configuration via the bus/interface controller 310. Some of the one or more output devices 338 include a graphics processing unit 340 and an audio processing unit 344, which are configured to communicate to various external devices, such as a display or speakers, via one or more A/V ports 342.

The one or more peripheral interfaces 346 may include a serial interface controller 350 or a parallel interface controller 352, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 348.

Further, the one or more communication devices 354 may include a network controller 356, which is arranged to facilitate communication with one or more other computing devices 360 over a network 104 communication link via one or more communication ports 358. The one or more other computing devices 360 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 332, the one or more removable storage devices 304, and the one or more non-removable storage devices 306 are examples of the computer-readable storage media. The computer-readable storage media is a tangible device that can retain and store instructions (e.g., program code) for use by an instruction execution device (e.g., the computing device 336). Any such, computer storage media is part of the computing device 336.

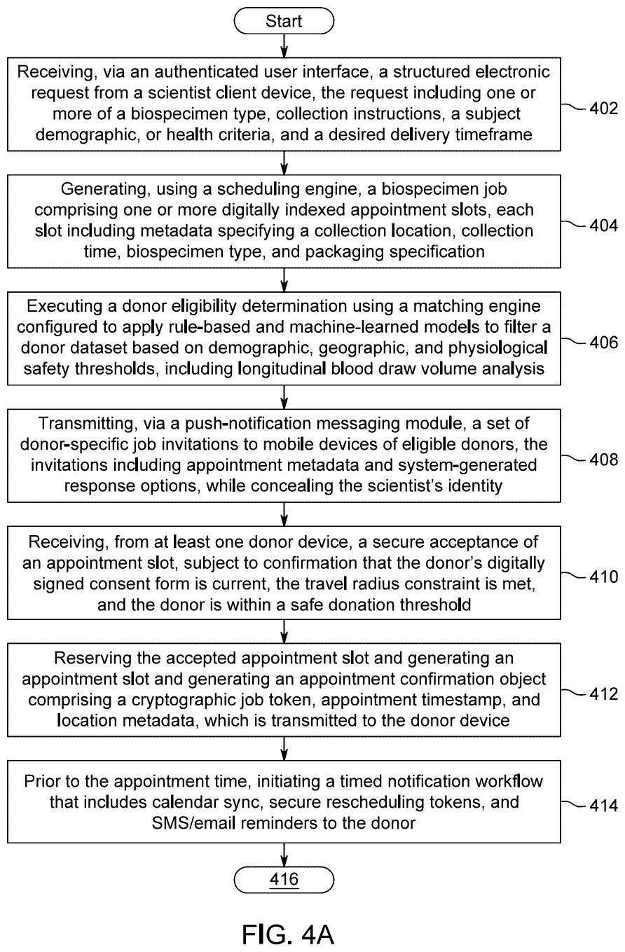

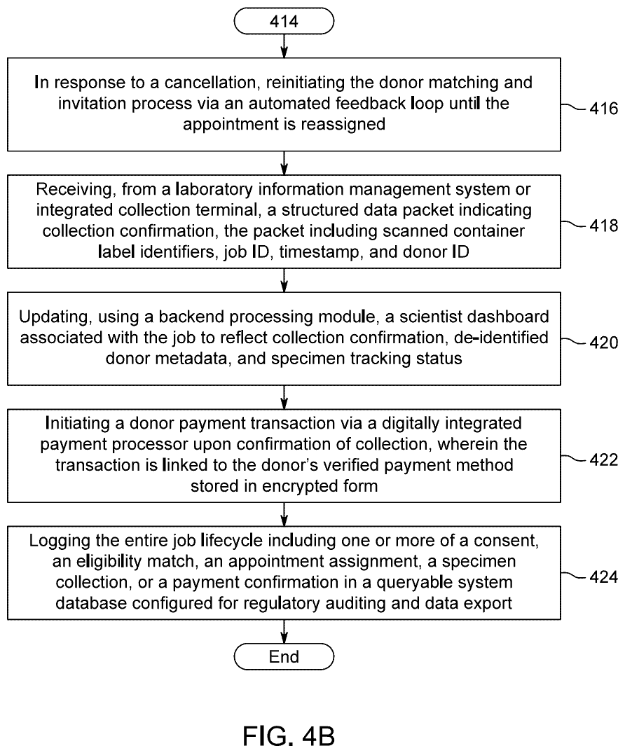

FIG. 4A illustrates a flowchart of an example computer-implemented method executed by a biospecimen procurement platform to coordinate the scheduling, matching, and confirmation of human biospecimen collection appointments. The method begins at a start operation and proceeds to block 402. In block 402, the system receives, via an authenticated user interface, a structured electronic request transmitted from a computing device operated by a scientist or researcher. The request includes one or more biospecimen types to be collected, such as whole blood, saliva, urine, stool, buccal swabs, skin samples, or other biological materials. The request further specifies collection parameters including handling instructions (e.g., temperature-controlled storage, type of collection tube), subject inclusion and exclusion criteria (e.g., individuals with or without a certain health condition), and a preferred delivery timeframe. The system is configured to ensure that the submitted request is securely stored and digitally linked to the requesting user's profile, which includes institutional affiliation and contact information for specimen delivery coordination.

In block 404, the platform generates a biospecimen job using a scheduling engine implemented by one or more processors. The job includes one or more appointment slots, each comprising structured metadata such as date and time of collection, collection site address or coordinates, the required biospecimen type, and additional processing details if applicable. In some implementations, the scheduling engine is further configured to communicate with external calendars or partner collection systems to check availability, ensure adequate staffing, and confirm courier pickup windows for time-sensitive specimens.

In block 406, the platform executes a donor eligibility determination using a matching engine. The matching engine applies a combination of rules-based filters and statistical or machine-learned classification models to compare the request criteria against a database of registered donor profiles. Each donor profile includes data such as age, biological sex, medical history, smoking and alcohol use, medication records, and geographic availability. The eligibility engine additionally calculates safe blood volume thresholds based on historical donation data, stored in time-indexed logs, to prevent donors from exceeding maximum volume limits within a predefined rolling window. For example, a donor who has already donated 400 ml of blood within the previous six weeks may only be eligible for a minimal additional volume in compliance with safety guidelines.

In block 408, the system transmits personalized job invitations to one or more eligible donor computing devices, such as mobile phones or web-based dashboards. The invitations include relevant job metadata, including location, date and time, biospecimen type, compensation, and any special instructions. These messages are delivered via one or more communication protocols, such as SMS, email, or in-app notifications, using a secure messaging interface. Donor invitations are anonymized with respect to the requesting scientist or organization to ensure participant privacy and compliance with human subjects research protections.

In block 410, the platform receives a secure acceptance of an appointment slot from at least one eligible donor. Before acceptance is finalized, the system confirms that the donor's digital consent form is on file and valid for the requested sample type, that the donor has affirmed availability for the specified location and time, and that participation would not exceed safe cumulative donation limits. The consent form may be completed through an integrated digital signature interface that verifies the donor's identity and records a timestamp of agreement. The platform may also display a checklist of conditions the donor must acknowledge (e.g., no recent illness or blood donation) before continuing.

In block 412, the system reserves the accepted appointment slot and generates an appointment confirmation object comprising a digitally signed identifier (e.g., a cryptographic token), the scheduled appointment time, and location metadata. This confirmation object is transmitted to the donor's user account or device and may be linked to a calendar application or displayed in a dashboard of upcoming appointments. The confirmation includes structured data fields that downstream components, such as label printers or check-in kiosks, can use to ensure correct specimen tracking and chain-of-custody.

In block 414, the platform initiates a timed notification workflow before the scheduled collection event. Notifications may include calendar synchronization, reminder messages, and secure links that allow donors to cancel or reschedule the appointment. These alerts are automatically generated based on the appointment metadata and may be sent at defined intervals (e.g., 24 hours and 1 hour before the appointment). If a donor cancels or does not confirm participation, the platform may automatically repeat the matching and invitation process described in earlier blocks to identify a replacement donor. Following block 414, the method proceeds to block 416, which transitions to specimen collection, labeling, delivery, and compensation processes, described in subsequent figures and sections of this specification.

FIG. 4B continues from the operations illustrated in FIG. 4A and describes subsequent stages of the computer-implemented method for coordinating biospecimen procurement and tracking within a digital matching platform. The method resumes after the donor has been scheduled and notified and proceeds to block 416. In block 416, in response to a donor cancellation or failure to confirm participation, the system reinitiates the donor matching and invitation process via an automated feedback loop. This loop uses previously stored eligibility results and job metadata to continuously search for alternative candidates until the vacant appointment slot is reassigned. In some embodiments, the platform may reference a dynamic backup pool of prequalified donors, ranked by predicted response probability, to accelerate reassignment. For example, if a donor cancels two hours before a scheduled 3:00 PM appointment, the system may automatically invite the next three highest-ranking backup donors in the same geographic area and sample eligibility class.

In block 418, the system receives confirmation of specimen collection from a laboratory information management system (LIMS) or a digitally connected collection terminal. The confirmation is transmitted as a structured data packet that includes one or more machine-readable container label identifiers, a unique job identifier, a timestamp indicating the moment of collection, and a donor ID associated with the collection. The container label identifiers may be scanned at the point of collection using a barcode or QR reader and are linked to the donor and appointment metadata stored in the platform's database. This block ensures closed-loop documentation of chain-of-custody from collection to delivery.

In block 420, a backend processing module updates a scientist-facing dashboard with the collection confirmation. The update includes de-identified donor metadata, such as donor ID, sample type, and collection time, as well as tracking status of the specimen (e.g., “collected”, “in transit”, or “delivered”). The dashboard may further display quality indicators or logistical notes, such as whether additional phlebotomy attempts were required. The backend module ensures that these updates are synchronized with the requesting scientist's user interface without revealing any personally identifying donor information.

In block 422, the system initiates a donor payment transaction through a digitally integrated payment processor, triggered upon receipt of confirmed collection data. The payment is linked to the donor's preselected payment method, such as a bank transfer, prepaid debit card, or third-party payment service, which has been previously verified and stored in encrypted form. For example, if the donor selected electronic bank deposit, the system performs an authenticated transaction through a secure payment APL In certain implementations, donors may receive a notification or receipt once the transaction is successfully posted.

In block 424, the platform logs the complete job lifecycle in a secure, queryable system database. The stored data includes, but is not limited to: the original consent document (digitally signed), donor eligibility screening results, appointment slot assignment records, collection timestamps, and payment confirmation. Each record is time-stamped and indexed using cryptographically secure identifiers. The database is designed to support regulatory auditability, data integrity validation, and reporting functions, and may be queried by authorized administrative users or compliance officers to export summaries for research oversight, reimbursement reconciliation, or IRB documentation. Upon completion of block 424, the method terminates, marking the end of the full biospecimen request and fulfillment lifecycle.

The disclosed system is a computer-implemented biospecimen procurement platform configured to coordinate the matching, scheduling, collection, and routing of human biological samples between registered researchers and qualified donors. The system utilizes non-transitory memory storing executable instructions and one or more processors configured to perform a series of data-driven and rule-based operations to enable real-time matching of donor criteria with scientist-generated biospecimen requests. The platform facilitates operational automation, dynamic logistics optimization, regulatory compliance, and end-to-end lifecycle tracking of each biospecimen request through integrated modules and APIs.

In one implementation, the platform includes a secure authenticated interface that enables a scientist to create a new biospecimen request via a structured data input form. The request includes one or more specimen types (e.g., blood, saliva, urine, stool, buccal or nasal swabs, skin tape, leukopak), metadata describing special handling or collection requirements (e.g., fasting status, volume, matched versus unmatched samples), and inclusion/exclusion parameters for donor recruitment (e.g., gender, age range, race or ethnicity, disease state, medication history, smoking status). The request also includes a desired delivery window and geographic constraints.

Upon receiving the structured request, the system executes a delivery feasibility engine that estimates the sample collection and routing timeframe by accounting for real-time donor availability, lab capacity, courier schedules, and backup recruitment logic. The engine generates a time-calibrated list of candidate delivery windows and presents these options to the scientist via the interface. Once the scientist confirms the desired timing, the system generates one or more biospecimen jobs. Each job corresponds to a unique combination of a donor, specimen types, and appointment logistics. If a matched sample set is requested (e.g., blood and saliva from the same individual), the system ensures both specimen types are collected within a synchronized collection window, Each job is automatically recorded in the system's scheduling engine and associated with appointment metadata including location, time, required sample types, equipment needs, and expected volume.

The platform then invokes a donor-matching engine configured to retrieve and evaluate a pool of potential donors whose stored profile data matches the criteria in the biospecimen job. Donor profiles include de-identified data points such as age, gender, geographic radius, sample preferences, health status, prior donation history, and donation cooldown periods. The platform may also evaluate historical donation volumes to compute whether the donor remains within a safe cumulative volume threshold, The matching engine applies deterministic filters and optionally a machine-learned ranking algorithm to score donor suitability and likelihood of participation.

Qualified donors are then invited to participate in the job via a secure messaging module that sends real-time notifications through email, SMS, or in-app channels. The job invitation includes appointment metadata such as time, location, compensation, and biospecimen types. When a donor accepts an invitation, the system reserves the appointment slot and removes it from future eligibility matching. The donor receives a calendar event confirmation that is digitally synchronized with external scheduling tools and includes embedded location metadata and routing instructions.

Leading up to the appointment time, the platform initiates an automated reminder workflow that transmits notifications to the donor at predefined intervals (e.g., 24 hours and 2 hours before the appointment). Donors may cancel or reschedule via a secure link embedded in the message, and such changes are processed by a rescheduling module that triggers an automated re-matching operation to fill the vacated slot. The system ensures continuity of recruitment by maintaining a real-time list of backup donors who can be automatically re-invited in the event of a cancellation.

At the time of the appointment, the donor presents at the collection site, which may include a laboratory, mobile phlebotomy unit, university clinic, or remote collection kiosk. Donor check-in is verified via manual ID confirmation or digital check-in terminals. The platform retrieves the job record and prints specimen container labels encoded with a job identifier, anonymized donor ID, collection timestamp, and sample type. These labels are affixed to each container, and the specimen is collected and marked as complete in the system using a digital input terminal or scanning station.

Once the sample is collected, a laboratory technician or site administrator confirms collection status in the platform, which triggers an update to the requesting scientist's dashboard. The update includes de-identified donor metadata, such as ID number, time of collection, and optionally, non-identifying health profile information as permitted by IRB protocol. The scientist receives an alert, via the preferred communication method, indicating whether the sample is en route (for external delivery) or available for pickup (for on-campus programs). The system supports integration with courier services to dynamically initiate pickup and delivery orders.

In some embodiments, following confirmation of specimen collection, the platform queues the donor for payment. A payment processor module initiates a transaction to the donor's verified and encrypted payment method. Acceptable payment methods may include bank transfer, reloadable debit card, or digital wallet. The system may enforce thresholds and verify delivery status before releasing funds. The platform logs the complete lifecycle of the job in a secure, queryable system database. Lifecycle records include the initial biospecimen request, matching results, appointment assignment, donor consent, collection confirmation, delivery metadata, and payment status. These records are time-stamped, digitally signed, and stored in a structured format to support audit trails, research reproducibility, and regulatory compliance. Reports can be generated dynamically by filtering dashboard data using structured queries. The reporting engine exports formatted reports in standardized file formats, including CSV, JSON, or Excel, enabling cross-system ingestion or research reporting.

The technical implementation described herein enables automated, rule-driven coordination of biospecimen logistics using structured computing operations, secure user authentication, dynamic calendar scheduling, and integrated data capture. The system improves over conventional manual specimen matching and collection methods by reducing response time, improving collection integrity, and enabling sample-specific routing and documentation workflows that scale with multiple concurrent users and job types.

The biospecimen procurement platform employs a role-based architecture comprising at least three primary user types: donor users, scientist users, and system administrators. Each user type interacts with the platform through authenticated, permission-controlled interfaces, which expose functionalities specific to their role. The platform is configured to enforce strict data partitioning and access control rules to preserve privacy and compliance with ethical and regulatory frameworks such as Institutional Review Board (IRB) guidelines.

Upon successful registration, a donor user is provisioned with a system-generated, de-identified donor ID composed of a randomly assigned alphanumeric string. This identifier is stored in association with the donor's internal system record but is not displayed to the donor at any point. The donor accesses a secure interface through which they may populate and maintain a structured donor profile. The donor profile includes age, self-reported gender, race, ethnicity, drinking and smoking history, and detailed health information collected through a structured health questionnaire. The donor may optionally grant access to third-party health data sources (e.g., insurance portals, laboratory systems, wearable health devices), subject to platform-supported data integration protocols.

The donor profile also includes sample eligibility preferences. For example, the donor may indicate willingness to provide blood, saliva, urine, stool, buccal swabs, or other biospecimen types. Each sample type is linked to donation-specific criteria (e.g., fasting status, volume requirements) that are enforced by the platform when matching donor eligibility to incoming specimen requests. The system further enables donors to digitally sign electronic informed consent forms through a secure module prior to participation in any collection appointment.

Donors may also input and manage availability constraints, including times of day, days of the week, and physical distance from which they are willing to travel to a collection location. Geographic availability is calculated using geofencing logic based on one or more stored address locations provided by the donor (e.g., home, work, school). These availability parameters are incorporated into the real-time donor-matching engine.

To ensure donor safety, the platform maintains a longitudinal log of donation activity. Each time a donor logs into their account, the system presents a user interface prompt querying whether they have donated or undergone a blood draw outside the platform since their last login. If the donor selects “yes,” they are prompted to enter the date, location, and nature of the procedure, such as a physician-ordered test, whole blood donation, plasma donation, or therapeutic phlebotomy. The platform automatically estimates blood volume based on the type selected (e.g., 10 ml for clinical blood test, 500 mL for blood bank donation) and subtracts that value from the donor's rolling eight-week volume threshold. If the donor exceeds the safe threshold, the platform programmatically suppresses biospecimen job invitations that would require further blood draws until the donor re-qualifies.

Donors may be eligible for at-home collection workflows depending on the sample type and job configuration. If so, the system generates a shipping kit request and dispatches the appropriate biospecimen kit to the donor. The donor may then collect the sample at home and either return it via designated carrier or present it at a drop-off location. Kit identifiers, collection instructions, and digital shipping labels are generated automatically through the platform and linked to the donor's job ID.

The donor-facing dashboard displays real-time updates on current and past jobs, each identified by date, sample type, location, and compensation. Donors can review upcoming appointments, manage preferences, cancel participation, or respond to new invitations. A dedicated job board displays opportunities matched to the donor's profile, ranked by relevance. For example, a donor with a rare disease profile may receive invitations filtered by study criteria and higher compensation levels. In some embodiments, donors with specific health conditions may enter a preferred compensation amount per sample type, which is stored and used in matching algorithms.

In some embodiments, scientist users interact with the platform via a secure, authenticated interface that includes a structured user profile, institutional affiliation, notification preferences (e.g., email or SMS), and payment configuration (e.g., purchase order, credit card). Scientists may associate individual biospecimen jobs with parent studies, enabling them to organize related collections under a unified research umbrella. Each scientist account includes a job history panel showing all current, completed, canceled, or duplicated specimen requests.

When placing a new biospecimen request, the scientist may input criteria such as sample type(s), matched or unmatched collection requirements, donor demographic and health filters, exclusion criteria, and desired collection and delivery timelines, and donor sample rating. After the job is processed and scheduled by the platform, the scientist receives real-time updates through their dashboard. Scientists may rate received samples on multiple criteria, including Sample Quality, Sample Handling, and Timeliness. These ratings are stored as sample-specific feedback and used to compute a composite donor rating that is visible to other scientists (in de-identified form) when filtering donor results. The rating system facilitates peer-based evaluation and helps maintain quality control over biological materials entering research pipelines.

The scientist's dashboard also includes a searchable, sortable donor table of all individuals who have participated in prior requests. This dashboard supports tagging and list management operations. For example, the scientist may tag certain donors as “favorites,” “exclude,” or “disease-specific cohorts” such as “type 1 diabetes,” “healthy controls,” or “post-COVID.” These tags may then be used to automate donor invitations in future jobs.

While the current system version restricts unsolicited donor browsing, the next iteration will include a secure search interface allowing scientists to query a national database of de-identified donors by age, race, health condition, medication use, donor sample rating, and location. Scientists can then tag promising profiles to a recruitment list, which may be activated by an administrator upon a future job submission.

Direct contact between scientists and donors is prohibited by system design. Donor identity, contact information, and job history are masked. In cases where a donor is to be recalled for longitudinal sample collection or special request, the scientist submits a request via the interface, which triggers a private outreach protocol handled by a system administrator. The administrator contacts the donor through approved channels (e.g., SMS, email, phone) and logs the outcome. If the donor accepts, the administrator schedules a new appointment and updates the original scientist job interface accordingly. From that point forward, the collection proceeds according to the standard platform workflow.

The biospecimen procurement platform includes a configurable role-based access control (RBAC) framework that defines a privileged administrative user type—referred to as an administrator or “admin.” Administrators interface with the platform via a secure, authenticated dashboard and are authorized to perform system-level operations beyond those accessible to scientist and donor users. The administrator role supports end-to-end platform configuration, user oversight, logistics management, and exception handling.

Upon login, an administrator is presented with an administrative control panel that includes modules for project management, donor and scientist account management, collection logistics, payment configuration, and audit reporting. Each module is backed by backend services that enforce access rules, log all changes, and trigger real-time platform updates.

Administrators can view and manage all biospecimen requests submitted by scientist users. Each request record includes metadata such as job number, sample types, donor filters, requested delivery dates, and current status (“draft,” “scheduled,” “in-progress,” or “completed”). The administrator can edit request parameters prior to scheduling, cancel requests, or update job routing logic as needed. For example, if a mobile collection unit becomes unavailable on a given day, the admin may reassign the job to a fixed-site location and notify the impacted donor(s) and scientist(s).

Administrators can also oversee the onboarding, verification, and deactivation of donor, pharma, individual pharma/research locations/campuses, and scientist accounts. For donors, this includes reviewing signed consent documents, verifying questionnaire completion, and activating eligibility based on medical safety thresholds. For pharma/research locations and campuses this includes creating a company and adding locations/campuses. For scientists, this includes verifying institutional affiliation and linking the account to a payment method, such as institutional billing, a credit card, or an external procurement platform. If a scientist attempts to place a request that violates system constraints (e.g., exclusion filters or delivery window overlaps), the platform may notify the administrator for manual review and override approval.

The admin interface also provides sample collection management tools. When a biospecimen job is in progress, the administrator can monitor the status of upcoming or completed collections. For on-site collections, the administrator may access a collection console that lists all scheduled jobs for a given day, sorted by location, time, and donor ID, The console provides real-time controls for marking specimens as “collected,” recording deviations (e.g., missed appointment, additional venipuncture attempts), and printing machine-readable labels linked to each sample container. If a specimen is collected manually, the administrator may scan the label and upload collection confirmation to update the scientist's dashboard.

Administrators are also responsible for managing special request workflows. For example, if a scientist wishes to recall a specific donor for longitudinal sampling, or if a job requires donors with highly specific profiles (e.g., “male, age 50+, former smoker, Parkinson's diagnosis”), the administrator may initiate targeted outreach. The administrator selects one or more pre-screened donors, sends communications via approved channels (e.g., SMS, email, or automated voice message), and schedules a customized appointment slot upon donor acceptance. The administrator can then reassign this slot to the existing job or create a new one with updated metadata.

The system provides tools for configuring the platform's operational parameters. Administrators can add new biospecimen types (e.g., synovial fluid, dried blood spots), associate them with handling protocols, and define allowable collection methods (e.g., venipuncture, self-collection, skin tape). The administrator can add or edit collection locations, whether fixed (e.g., a hospital lab) or dynamic (e.g., mobile phlebotomist route stops). Each location is configured with operating hours, staffing capabilities, and equipment availability.

Payment configuration is also managed by the administrator. The admin can define compensation rules for each specimen type based on volume, rarity, or special handling. For instance, whole blood donations may be compensated at $50, while stool samples from donors with irritable bowel syndrome may be set at $100. These values are entered into the system's compensation matrix, which dynamically calculates donor payouts based on job metadata and payment preferences. Payment rules may also include bonuses for repeat participation or rapid scheduling.

Administrators can control the user-facing messaging displayed throughout the platform. This includes modifying invitation text, email templates, reminder notifications, and dashboard tooltips. Each message template can be updated using a WYSIWYG editor or JSON configuration file and may be customized by role, event type, or collection context. For example, a reminder email sent 24 hours before a saliva sample may include specific hydration instructions, while a post-collection message may include compensation and tracking updates.

To support compliance and scalability, administrators can delegate tasks by adding additional admins with tiered permission levels. For example, a regional admin may be restricted to managing donors and collections in a specific state, while a super-admin has global access across the platform. Each admin account is logged, and all actions are traceable in an audit trail module, which provides immutable logs of edits, user messaging, consent updates, payment transactions, and sample handling events. In some embodiments, the administrator role is implemented through a combination of access-controlled modules, event-driven workflows, data validation rules, and integration layers that enable operational continuity, compliance, and customization across all facets of the biospecimen procurement process. The system architecture ensures that all administrator actions result in real-time platform updates and are captured in a secure, queryable database for later reporting and review.

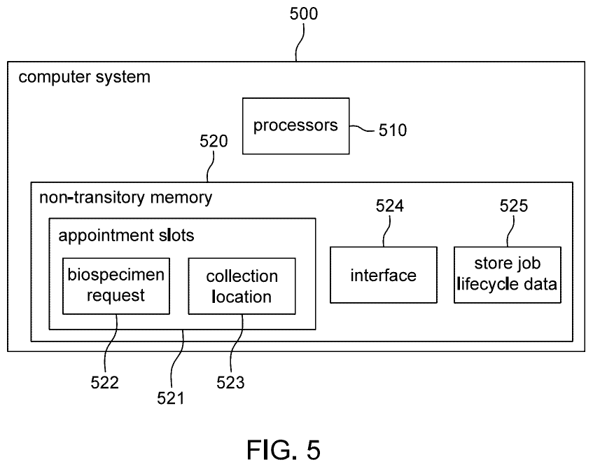

FIG. 5 illustrates an example system architecture of a computer system 500 configured to support the biospecimen procurement platform described in the present disclosure. The computer system 500 includes one or more processors 510 operably coupled to a non-transitory memory 520. The computer system 500 may be implemented in a server environment, a distributed cloud infrastructure, or a virtualized instance accessible over a secure network by scientists, donors, and administrators.

The one or more processors 510 may include central processing units (CPUS), graphics processing units (GPUs), digital signal processors (DSPs), application-specific integrated circuits (ASICs), or any combination thereof. The processors 510 are configured to execute software modules and machine-executable instructions stored in the non-transitory memory 520, including instructions for managing specimen requests, donor matching, appointment scheduling, and job lifecycle tracking.

The non-transitory memory 520 stores multiple functional components, including a data structure 521 for managing appointment slots. Each appointment slot represents a discrete opportunity for biospecimen collection and includes associated metadata fields. For instance, a biospecimen request 522 may specify the desired sample type (e.g., blood, saliva, urine), processing details (e.g., centrifuge within 30 minutes, cold-chain transport), demographic or health eligibility filters (e.g., healthy individuals aged 18-45), and compensation parameters. A corresponding collection location 523 may define a physical address, geographic coordinates, or facility identifier for the collection event, such as a third-party laboratory site, university clinic, or mobile phlebotomy point-of-care station.

An interface module 524 provides the functionality to receive and process inputs from various user roles. For scientists, the interface enables submission of new specimen requests, review of appointment confirmations, access to dashboards showing de-identified donor metrics, exportable de-identified donor health information, and exportable reports to be used in an Electronic Lab Notebooks. For donors, the interface provides a secure login, displays matched job invitations, and allows for consent, acceptance, rescheduling, and payment preference management. The interface may support delivery through a web portal, native mobile application, or hybrid cross-platform framework, and may incorporate accessibility and localization features.

The memory 520 also includes a job lifecycle storage module 525, which is configured to store structured records for each stage of the biospecimen collection process. These records may include digitally signed informed consent forms, donor eligibility assessments, appointment confirmations, collection timestamps, sample verification metadata, delivery status, and payment transaction logs. Each record is stored in a queryable format, allowing the system to support data export, regulatory audit generation, and longitudinal study tracking. In some embodiments, the lifecycle data may be stored in a relational database, document-oriented store, or append-only log structure depending on performance and compliance requirements.

In certain configurations, the computer system 500 communicates with external systems, including laboratory information systems, calendar synchronization services, courier dispatch platforms, and payment gateways, via secure application programming interfaces (APIs). These integrations may support real-time updates of specimen status, electronic delivery confirmations, and automated compensation disbursements to donors upon verified collection, The architecture shown in FIG. 5 provides a modular and scalable foundation for enabling secure, real-time coordination between multiple stakeholders involved in human biospecimen research while enforcing privacy, safety, and compliance protocols.

In some embodiments, the marketplace configuration of the biospecimen procurement platform supports decentralized, high-throughput sample acquisition through intelligent scheduling, automated donor overbooking, and direct system integrations with external collection and logistics providers. By leveraging third-party infrastructure and structured data exchange, the platform enables scientists to access fresh, high-quality human biospecimens while ensuring safety, traceability, and privacy for all participants.

In another embodiment, the biospecimen procurement platform is implemented as a distributed marketplace system designed to connect scientists and research institutions with qualified public donors through a secure, cloud-hosted infrastructure. The platform operates across a network of third-party collection sites and mobile service providers, dynamically allocating jobs and managing biospecimen logistics using programmable APIs, rules-based donor matching, and real-time system monitoring. Key technical features are parameterized using defined ranges to ensure operational reliability, data integrity, and biological sample viability.

Identity verification for both donors and scientists is completed through multi-step authentication workflows, with verification latency maintained within a range of approximately 1.5 to 4.0 seconds. This range enables rapid onboarding while accommodating variation in third-party verification response times and ensuring compliance with security protocols. Each registered user is assigned a unique system ID, and donors receive a de-identified hashed alphanumeric identifier of between 12 and 36 characters to protect privacy.

Donor recruitment is radius-constrained to improve collection efficiency and donor compliance. The platform allows the system administrator or job-routing algorithm to define a donor radius ranging from 5 to 100 miles from an active collection site. For example, a default range of 25 to 50 miles can strike a balance between donor participation and sample timeliness, while ranges narrower than 10 miles may be preferred in urban centers with dense donor populations. Larger radii, such as 60 to 100 miles, may be selectively invoked when matching rare phenotypes or when urgent study timelines require broader reach.

To mitigate the impact of donor no-shows, the platform applies a configurable overbooking factor ranging from 1.1× to 2.5× depending on sample type, historical attendance rates, and time sensitivity. For common, non-invasive samples such as saliva or urine, a lower overbooking threshold may be used. However, for high-urgency or multi-sample collections, such as matched stool and blood from a specific demographic, the system can apply an overbooking ratio closer to 2.0×. This strategy reduces missed delivery windows and helps ensure biospecimen supply chain integrity without human intervention.

Sample viability is maintained through enforceable buffer times and temperature-aware packaging logic. For ambient-stable samples such as skin swabs or saliva, the platform supports delivery timelines ranging from 12 to 72 hours post-collection. For temperature-sensitive samples such as whole blood or plasma, the system requires delivery within 2 to 12 hours. In such cases, thermal packaging is dispatched with cold-chain buffer capacity of 1 to 3 hours to account for transit variability. These timing controls ensure the collected biospecimens remain usable for downstream molecular, proteomic, or microbiome analyses.

When donor matching is initiated, the rules-based engine applies filtering logic across 10 to 50 health and lifestyle fields, including BMI, smoking history, drinking frequency, medical conditions, and medication use. Donor age ranges can be specified from 18 to 85 years, and health constraints may include minimum lab values (e.g., hemoglobin >13.0 g/dL), recent medication exclusions (e.g., no immunosuppressants in the past 30 days), or positive disease status (e.g., confirmed autoimmune diagnosis). A matching score threshold of 75% to 90% is applied to determine eligibility. A higher threshold (290%) may be used for rare disease studies or specialty biomarker trials to maximize data quality, while lower thresholds (75-80%) allow broader access for control group studies or time-sensitive jobs.

Compensation offered to donors varies by sample type and complexity, ranging from $15 to $500. For example, routine swabs or saliva collections may be compensated in the range of $15 to $30, whereas multi-sample matched draws or collections involving rare disease patients may reach compensation levels between $100 and $500. These incentives are dynamically managed by the platform and tied to donor compliance and show rate metrics. Payment disbursement occurs through digital channels such as direct bank transfer, electronic payment methods, or mailed checks, and is released within a latency window of 1 to 3 business days post-verification. This timing window is optimized to encourage future donor engagement while maintaining fraud prevention safeguards,

At-home collection is offered via mobile phlebotomy when in-clinic collection is not feasible. The dispatch system assigns collectors based on travel radius (up to 30 miles), daily appointment volume (typically 4 to 10 jobs per collector), and donor scheduling availability, which can range from tight 15-minute windows to flexible 2-hour blocks. Each mobile phlebotomist uses a handheld interface to receive job information, scan pre-printed sample labels, and confirm collection through barcode-based verification. Sample pickup is coordinated with courier dispatch or local lab drop-off within a 1- to 3-hour post-collection window to maintain compliance with sample integrity requirements.

Real-time system operations are monitored across the platform's web-based dashboards for donors, scientists, and administrators. Status transitions such as job creation, donor match, appointment acceptance, sample collection, and delivery are timestamped with latency goals under 10 seconds. For high-volume integrations (e.g., national labs), the system supports throughput of 20 or more API transactions per second with data payloads under 512 kilobytes, while boutique labs and mobile fleets operate on lower-throughput endpoints (1 to 5 TPS). Each transaction is recorded with an immutable audit trail that includes timestamps, IP addresses, user session identifiers, and cryptographic hashes.

All data transfers, donor health record access, and sample lifecycle logs are retained for a minimum of 7 years, extendable to 15 years based on customer or regulatory requirements. These parameters support compliance with HIPAA, CLIA, and GDPR, ensuring that specimen sourcing is conducted ethically, securely, and traceably. This level of parameterization and range specificity provides technical benefits including improved fulfillment reliability, donor safety, operational scalability, and biospecimen quality control, thereby supporting the platform's utility for translational and clinical research at scale.

In an embodiment, the system provides a web-based donor interface configured to securely onboard, verify, and engage participants for human biospecimen donation using a range of programmable thresholds and eligibility constraints. Identity verification is conducted using a third-party authentication service supporting variable verification depths. For example, identity resolution is accomplished using a minimum of two and up to five data points, including but not limited to full name, date of birth, last four digits of a government-issued identification number, address, and mobile number. The identity verification process is designed to complete in a latency window ranging from approximately 1.2 seconds to 4.8 seconds, balancing fraud detection sensitivity with user onboarding efficiency. The system is further configured to support tiered identity checks with escalating verification based on user risk scoring or donation value thresholds.

Upon successful onboarding, each donor is issued a persistent, non-sequential alphanumeric donor ID ranging in length from 10 to 36 characters, with checksum validation for error detection. Subsequent logins require multi-factor authentication (MFA), with options including one-time passcodes via SMS, app-based authentication, and biometric options on supported devices. The system enforces a session expiration time between 10 and 60 minutes of inactivity, with session token rotation every 3 to 15 minutes to enhance data security and prevent token reuse in case of interception.

The donor profile interface displays editable personal fields and system-locked verified fields. Editable ranges include contact methods (1-3 registered email addresses and phone numbers), up to 5 donation locations with radii configurable from 1 to 100 miles, and time-based location scheduling allowing donors to designate specific weekdays or time windows (e.g., 8:00 AM to 12:00 PM) for each location. Biological sex is selected from predefined options, and optional gender identity attributes allow for user specification with inclusive options. Ethnic and racial backgrounds may be selected using a dual-heritage model supporting between 1 and 4 selections per donor.

The health questionnaire includes 20 to 150 structured fields covering diagnosed conditions, medications, allergies, family history, recent exposures, and behavioral health attributes. To ensure longitudinal tracking, the questionnaire may be updated at intervals ranging from every 30 days to every 180 days. Donors who fail to update their health history within the configured interval are automatically excluded from matching algorithms until compliance is restored.

Donors indicate which biospecimen types they are willing to donate by selecting from a checklist of up to 25 distinct types. These may include, but are not limited to, blood (whole blood, plasma, PBMC), leukopaks, saliva, urine, stool, nasal swabs, buccal swabs, skin tape, and breath condensate. The system supports opt-in/opt-out toggles for each sample type with real-time eligibility calculations, and may enforce upper limits per type, such as no more than 500 mL of blood every 8 weeks or a maximum of 5 stool donations in a rolling 30-day window. Each biospecimen type has a defined recovery window and safety margin, both of which are enforceable system parameters and may be configured by an administrator per IRB guidance or medical advisories.

Payment preferences are configured in the donor's profile, with the system supporting up to four linked payment options per user, including direct deposit (ACH), electronic payments, and/or physical check. Payment disbursement latency is configurable between 1 to 5 business days. For high-value collections or recurring donors, fast-track payment options (e.g., under 24 hours) may be selectively enabled, subject to account verification and transaction limits (e.g., <$250 per job). Donors may also define a preferred minimum compensation threshold for each biospecimen type (e.g., a donor may specify that they will only accept blood jobs paying $50 or more), thereby filtering visible job opportunities.

Donors access a job opportunity dashboard filtered by criteria including job date, sample type, location, and compensation. Sorting may be applied across 2 to 10 fields, with filters allowing for radius-based location preferences (1-100 miles), date range windows (e.g., next 3 to 30 days), and compensation minimums ($10 to $500). Each job includes a structured job card with time, location, sample type, compensation, and collection method. Job acceptance includes a digital acknowledgement process requiring the donor to check off between 3 and 10 eligibility affirmations before finalizing participation.

The “Upcoming Donations” view displays a paginated list of all future accepted appointments. Each entry includes a job number (up to 16 characters), scheduled date and time, location address, expected sample type(s), and compensation. The system allows appointment modifications up to 6 hours prior to collection time, subject to administrative override rules. Donors may reschedule or cancel appointments via a dropdown action menu, and the system automatically requeues declined slots for backfill using active donor matching logic.

Donation history is retained and visible across a rolling 24-month period, and includes status indicators such as “complete,” “canceled,” “no-show,” and “paid.” A visual blood donation tracker displays a running total of blood donated over the last 8 weeks, with a hard safety threshold of 500 mL enforced by the platform for healthy donors. Donors with chronic conditions may be flagged by administrative rules to limit donation volume further—for example, to 300 mL over 8 weeks for individuals with diabetes or low hemoglobin.

Each time a user logs into the system, the interface may present a recall check prompt asking whether they have donated blood elsewhere since their last session. A donor answering “yes” is presented with a short intake form capturing the type (doctor's test, plasma donation, blood bank), amount (range of 10 mL to 1000 mL), and date of donation. The system then recalculates safe eligibility based on this self-reported data and updates the donor's profile with an estimated deferral window, which may range from 1 to 90 days depending on volume, sample type, and condition.

The data displayed and modified via the donor interface is logged using an auditable, immutable backend log. All donor profile edits, job acceptances, cancellations, and health disclosures are timestamped to the nearest second, and linked to the donor's session ID and originating IP address. This log is retained for a configurable duration of 7 to 15 years to meet compliance with HIPAA, GDPR, and IRB auditability. Through these structured fields, variable ranges, and tiered eligibility logic, the donor interface provides a robust technical framework for managing diverse user populations, protecting participant health, and enabling secure, IRB-compliant biospecimen procurement workflows at scale.

In one embodiment, the platform includes a dedicated scientist-facing interface for managing sample procurement workflows, communication preferences, and donor tracking. The scientist profile settings module enables the scientist to configure core account information including their name, organizational title, mobile number (used for SMS alerts), delivery address, direct office phone number, group affiliation name, and an internal group identifier. These profile values are stored in secure, editable fields and are validated before sample orders can be submitted.

The alerts configuration module allows scientists to customize their communication preferences. Scientists may select which categories of system-generated notifications they wish to receive—such as sample readiness, delivery confirmations, or schedule changes—and can define preferred delivery channels including SMS, email, or platform notifications. Alert frequency can be set at immediate, hourly, or daily intervals.

The sample request dashboard serves as the central command module for managing biospecimen procurement. This interface displays all active and historical sample requests associated with the scientist's account Each row in the dashboard includes a job number, project name, delivery date, biospecimen types requested, and current job status (e.g., scheduled, draft, canceled, completed). The dashboard is sortable by column headers and supports data export in Excel or CSV format for reporting and compliance use. The interface also includes three-dot controls enabling the scientist to view job details, edit unsubmitted jobs, cancel pending jobs, or duplicate a prior job. A “New Request” button at the top of the page links to the sample request creation module. A summary snapshot is also presented at the top of the page, showing real-time counts of upcoming scheduled requests and confirmed appointments.

To initiate a new job, the request form interface prompts the scientist to validate their profile information to ensure accurate matching and delivery. In the project details section, the scientist may associate the request with a previously created Parent Study. If applicable, the system allows configuration of repeat donor participation. The scientist then inputs a project name, number of volunteers needed (typically ranging from 1 to 100), and an institutional purchase order number if required. The general donor requirements module allows the scientist to specify eligibility criteria for the requested biospecimens. Input fields may include donor age range, biological sex, race or ethnicity, smoking or alcohol history, medication usage, and general health status. In the current system, donor eligibility is based on self-reported information; future versions will support integration with lab test data and longitudinal health profiles.

In the sample specifications module, scientists define the biospecimen type (e.g., blood, urine, stool, saliva), collection volume, processing method, and collection container type. Optional inputs include collection instructions such as centrifuge protocols, refrigeration, or chemical stabilization. Scientists may optionally request or exclude specific donor IDs based on prior study participation. When list management functionality is implemented, scientists will be able to select donors from curated lists such as “Favorites,”“Healthy Donors,”or disease-specific cohorts.

Prior to scheduling, the scientist is prompted with a sample conditions acknowledgment screen. Here, they certify understanding of the sample handling constraints and compliance policies by checking a digital signature box. From this screen, the scientist may proceed to schedule the request or save it as a draft. The scheduling interface displays a calendar of available delivery dates and times, dynamically generated based on sample type, donor availability, processing time, and transportation constraints. In special request scenarios, where individual donors are targeted for participation, a platform administrator may initiate a separate scheduling workflow. The administrator will coordinate directly with the donor to determine a mutually acceptable time and location, such as a collection site, mobile phlebotomy visit, or at-home test kit, Once confirmed, the system will update the scientist's dashboard with the finalized sample delivery details.

The Parent Studies dashboard provides a summary of all umbrella studies associated with the scientist. This view displays parent study titles, dates created, the number of linked projects, owner name, and status (active, paused, or archived). Scientists may create, edit, pause, or delete parent studies from this dashboard. The interface also enables the scientist to drill down into individual sample requests associated with each parent study. The list management module (to be released in a future version) will allow scientists to build, name, and organize donor lists such as “Exclude from All Studies,” “Type 2 Diabetics,” or “Repeat Donors.” These lists will be available as selectable filters during the sample request creation process.

The donor dashboard provides a structured view of all de-identified donors who have participated in the scientist's sample requests. Scientists may sort by donor ID, gender, race, sample type, and donation date, and may export health histories where authorized. Donors can also be tagged for list assignment or recall. The payment configuration module offers scientists several options depending on the institutional setup, including Purchase Order, credit card, or third-party marketplace billing through one or more third party integrated services, Payment terms and pricing structures are defined during scientist onboarding and enforced throughout the job lifecycle. In some embodiments, the system includes a session management module that permits scientists to sign out manually or automatically after a configurable period of inactivity. All scientist activities are audit-logged with timestamps to ensure compliance with regulatory frameworks and internal research governance policies.

In some embodiments, the administrator interface of the Donor X platform enables secure and efficient management of all operational activities associated with biospecimen request fulfillment and donor coordination. The administrator maintains a profile record that includes a verified mobile number for SMS notifications, with authentication protocols in place to ensure secure access. As a control mechanism to preserve data integrity and system segregation, the platform prohibits administrators from simultaneously registering as donors.

The administrator's main interaction point is the project dashboard, which presents a real-time, dynamically updated overview of all biospecimen jobs. Each record includes structured fields such as expected delivery date, number and type of required samples, number of participating donors, the pharmaceutical entity name (in the marketplace configuration), institutional campus (in both on-campus and marketplace configurations), lab location, and the identity of the requesting scientist. This structured interface supports advanced filtering, column-based sorting, and on-demand export in machine-readable formats such as CSV or Excel, facilitating downstream reporting and compliance tracking. Each job is also associated with a persistent and unique job identifier, enabling traceability and eliminating the need for ad hoc manual coordination. From this interface, the administrator can trigger additional actions using an integrated control element (e.g., a three-dot menu) to access functions such as job detail viewing, sample collection initiation, cancellation of unfulfilled requests, and marking a job as “picked up.” This embedded control structure reduces the number of interface transitions, enhancing system efficiency and minimizing human error in data management.

When initiating a sample collection, the administrator is guided through a workflow that ensures regulatory compliance and sample integrity. Upon selecting the “collect samples” action, the platform routes the administrator to a secure job detail page. In marketplace implementations where automatic donor-to-job linkage is not feasible due to third-party lab constraints, the platform allows for manual donor selection from a verified dropdown list. In the marketplace implementation where automatic donor-to-job linkage is feasible, upon collection of samples by a phlebotomist/tech, the sample collection is automatically updated via an encrypted message returned from the third-party lab. This list contains real-name identifiers only visible to administrators, ensuring privacy of donor identities from scientists. Once a donor is selected, the platform enables the administrator to annotate punctuality (on-time or delayed), initiate label printing routines (once enabled), and view specific collection protocols tailored to the job. The administrator can mark a sample as collected and optionally input metadata such as the number of venipuncture attempts or procedural deviations. These annotations are stored in a secure audit log accessible only by authorized administrators, ensuring accountability while preserving researcher blinding. In on-campus configurations, donors may receive variable compensation based on the complexity or difficulty of collection (e.g., multiple needle sticks), which is managed through this interface,

Notification workflows differ across implementations to reflect the nature of sample logistics. In the on-campus version, the administrator retains manual control over when a scientist is notified that samples are ready for pickup, allowing coordination based on internal lab availability. In contrast, the marketplace version utilizes automated notifications integrated with external courier APIs. When the courier scans the sample at pickup, the system programmatically generates a timestamped alert to the scientist along with a trackable shipment link. This automation reduces manual coordination overhead and improves timeliness and reliability of delivery.

The donation reports module allows administrators to view all completed biospecimen collections for reconciliation and payment. The interface supports data sorting and export, enabling downstream integration with financial management systems. In one implementation, the platform includes Plaid integration, allowing donors to securely link their bank accounts. Upon marking a sample as collected, the platform schedules a direct payment to the donor's linked account at the close of the business day This real-time payment scheduling represents a technological improvement over traditional manual reimbursement workflows, reducing administrative overhead and accelerating donor engagement. In the marketplace model, if automated payment is not enabled, the administrator can export donor reports for processing through external payroll systems.

The donor management module allows an administrator to access, sort, filter, and export records of all enrolled donors. In the on-campus configuration, this list is restricted to institution-affiliated donors; in the marketplace configuration, it includes public donors who have completed identity verification and informed consent. Each donor profile includes metadata such as activity status, eligible sample types, payment history, and job participation. Administrators may also invite donors to participate in specific jobs via the integrated action menu, or deactivate profiles that no longer meet eligibility criteria. Access to this module is gated behind administrator roles to ensure compliance with data handling regulations and maintain system security.

Pharmaceutical and campus-level management modules allow the system to be configured hierarchically, On-campus administrators can define individual campuses, appoint campus-level managers, and onboard scientists to request specimens. Marketplace administrators perform analogous actions at the pharma account level. During setup, administrators can configure pricing models, volume discount tiers, and acceptable payment methods. For example, if credit card transactions are enabled for a pharma entity, the associated payment APIs are activated to permit checkout during job submission. This dynamic configuration of transactional pathways enhances the platform's flexibility and enterprise readiness.

Appointment durations and compensation amounts are also fully configurable through the administrative panel. In the on-campus configuration, an administrator defines the expected appointment length and donor compensation for each sample type. These values may reflect monetary amounts, redeemable points, or other incentive models aligned with organizational policies. In the marketplace, sample appointment durations are constrained by third-party lab schedules, while compensation amounts are tiered based on sample type, disease-state relevance, or whether a donor is being recalled. These settings are tied to sample logistics optimization algorithms that maximize donor availability while avoiding over-sampling.

The platform includes an admin management module through which super administrators may provision additional admin accounts. Each admin account may be granted permission-based access to specific modules such as donation reports, donor management, lab configuration, and email/SMS messaging. This role-based access control framework enforces separation of duties, enhances operational security, and ensures compliance with institutional policy and regulatory requirements. Messaging configuration is handled through a dedicated module that enables editing of all email and SMS templates used throughout the platform. Administrators may update the subject line, body text, tokens, and delivery logic of each message to match organizational tone or regulatory messaging standards.

In some embodiments, lab settings are maintained through a geographic and metadata-based interface. On-campus lab locations are linked to their associated campuses. In the marketplace implementation, lab partners such as hospitals, diagnostic labs, or outpatient centers are registered with address and geolocation data, enabling donors to receive automated navigation links at the time of appointment. These details improve real-time appointment adherence, reduce missed collections, and enhance the efficiency of sample delivery routing.

From a technical standpoint, the administrator interface described herein offers tangible improvements to biospecimen procurement workflows through dynamic record generation, automated donor-scientist separation, real-time courier integration, secure banking integration, and hierarchical role-based access. These improvements are not abstract ideas but specific technological enhancements that transform how biospecimen collection is managed across disparate systems, yielding improvements in scalability, reliability, and data integrity.

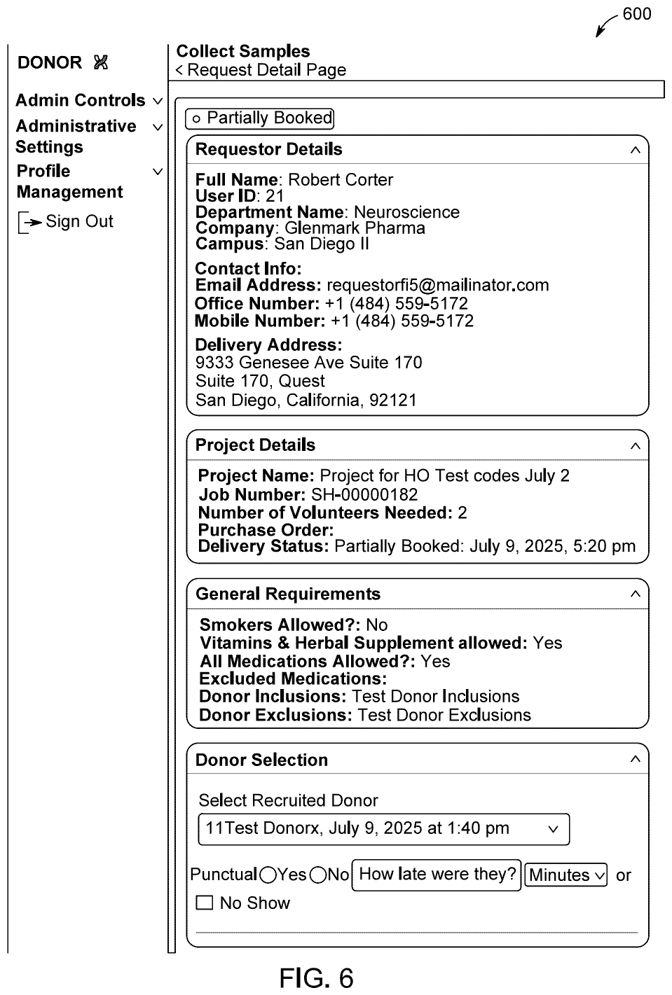

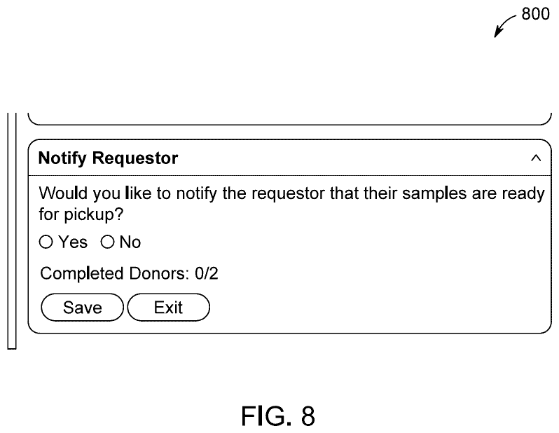

FIGS. 6-24 illustrate various example user interfaces of a multi-platform biospecimen collection and fulfillment system. The system facilitates the recruitment and scheduling of qualified biospecimen donors based on criteria set by scientists or research organizations. The interfaces depict both the administrative and participant-facing functions of the platform, including order creation, sample-type selection, appointment scheduling, donor engagement, sample collection, delivery, and post-collection data reporting.

FIGS. 6 through 24 collectively illustrate components of a computer-implemented system designed to optimize the collection, routing, and delivery of human biospecimens for research purposes. The system supports use cases in both on-campus implementations and a public-facing marketplace environment. A primary feature of the platform is the ability to match donors with biospecimen requests placed by scientists, ensuring sample freshness and timely delivery through real-time scheduling, smart logistics, and identity-secured workflows.

In operation, a scientist initiates a specimen request through a graphical user interface (GUI), specifying one or more parameters including desired biospecimen types (e.g., blood, saliva, urine, stool, swabs, skin tape, leukopak), donor demographic filters (e.g., age, gender, race), health history criteria (e.g., diagnosed conditions, medication use), and special instructions (e.g., fasting, matched sampling across timepoints). Upon request submission, the system executes a timing algorithm that calculates feasible sample delivery windows, factoring in parameters such as donor pool availability. location-based logistics, sample collection times, and transit times.

Each approved request generates a discrete job object in the system, which is scheduled for fulfillment through automated calendar integration. For instance, a request requiring four sets of matched blood and saliva samples from four unique donors would generate four independent jobs, each pre-scheduled and presented as an available opportunity to eligible donors. The platform includes matching logic to identify and invite eligible donors based on compliance with predefined filters. Donors may view job opportunities, review terms, and confirm availability. Upon acceptance, the system locks the selected appointment and issues a calendar event containing the scheduled location, time, and sample type.

Leading up to the collection window, the system automatically issues reminders and provides a donor-controlled interface to reschedule or cancel appointments. If a cancellation occurs, the system executes a recursive invitation logic to fill the vacant slot by re-engaging other qualified donors. Donor identity is verified at the point of collection, which may involve manual or digital check-in mechanisms. The system then generates collection-specific labels encoding metadata such as donor ID (de-identified), job number, date, and time. Upon successful collection, the samples are registered in the system and marked accordingly.

Post-collection, the system updates the scientist's dashboard with real-time status and de-identified donor metadata, including relevant health history attributes. Depending on the implementation (on-campus vs. marketplace), the samples are either delivered to the scientist's location via courier integration or made available for on-site pickup. Notification delivery methods (e.g., SMS, email) are configurable by user preference.

After sample processing, donors are added to a compensation queue. Payment is executed using preferred digital disbursement methods (e.g., direct deposit, peer-to-peer platforms), while institutional clients are billed based on preconfigured payment terms and pricing structures. Additionally, the system provides dynamic reporting capabilities that allow for exportable analytics based on job metrics, donor engagement, and fulfillment statistics, supporting compliance documentation and operational transparency.

### Scientist Places Order

A scientist authenticated through the system submits a biospecimen request using a graphical user interface that allows selection of required sample types, including but not limited to blood, saliva, urine, stool, buccal swabs, nasal swabs, skin tapes, and leukopaks. The interface permits input of demographic filters (e.g., gender, age, race), health conditions, medications, and inclusion/exclusion criteria. Additionally, matched sample logic may be applied, where multiple sample types are collected from the same donor within a designated collection window.

### Sample Times Are Calculated

Upon submission of a biospecimen request, the system performs time estimation based on donor availability, lab throughput, and courier logistics. An optimization algorithm computes the estimated time required for sample acquisition and delivery, and generates a list of valid fulfillment windows, which are presented to the scientist as selectable delivery time options.

### Job is Created

The request is automatically parsed into one or more biospecimen collection jobs. For each donor required, the system generates a discrete job with its own appointment time. These jobs are integrated with phlebotomist calendars and lab capacity constraints to ensure efficient sample collection. Jobs are linked to a tracking ID and include metadata such as donor eligibility criteria, requested sample types, and collection timing.

### Donors Invited

The matching engine queries the donor database for individuals whose profile attributes satisfy the criteria specified in the job request. Qualified donors receive invitation notifications through their preferred communication channel. If a donor accepts a job, the system removes that time slot from the available pool and issues a confirmation with time, location, and sample instructions.

### Donor Cancel/Change Appointment

Leading up to the scheduled collection, donors may cancel or reschedule appointments via the donor-facing dashboard. The system monitors cancellations and automatically invites alternate qualified donors from the waitlist or backfill pool to maintain appointment integrity.

### Sample Collection

At the scheduled time, donors present at the designated collection site where their identity is verified through manual or electronic means. The administrator accesses the job interface, selects the donating individual, and triggers label generation, which includes donor ID, job number, collection date and time, and sample metadata. Collected samples are labeled and confirmed in the system as “collected.”

### Scientist Dashboard Updated

Upon confirmation of sample collection, the scientist's dashboard is updated with a de-identified donor identifier and relevant health information as permitted by the original consent. The job status is updated, and the delivery pipeline is initiated.

### Samples Delivered/Available for Pickup

The system determines the delivery method based on deployment configuration. In marketplace environments, courier integration is triggered via API, and tracking information is transmitted to the scientist. In on-campus implementations, the system notifies scientists that samples are ready for in-person retrieval.

### Donors Paid

Upon collection confirmation, donors are queued for compensation. Payments are processed using preferred disbursement methods, including direct deposit, peer-to-peer payment tools, or paper check, depending on system configuration and user selection.

### Donor Samples Rated

Upon receipt of samples, the requestor/scientist has the ability to rate the samples they have received against a set of criteria that will contribute to a donor's sample rating that is used in the matching algorithm.

### Sample Payment

Sample payment workflows follow institution-specific billing logic. The platform generates invoices based on the per-sample pricing associated with the ordering scientist or department.

### Dynamic Reporting

Administrative and user dashboards support real-time data filtering and export of biospecimen collection metrics. Reports may include donor activity, sample throughput, punctuality, and fulfillment timing. Output formats include structured spreadsheet files for external analysis and compliance reporting.

FIG. 6 illustrates an example user interface 600 of a sample collection coordination module within the administrative platform. The GUI is configured to enable an authorized user (e.g., an administrator or coordinator) to view, manage, and finalize details associated with an active sample collection request. The interface comprises a vertically structured layout including multiple collapsible sections for efficient information display and task execution. The left-side navigation pane provides selectable options including Admin Controls, Administrative Settings, and Profile Management, along with a sign-out control, allowing navigation across different areas of the platform.

The primary content pane displays a page titled “Collect Samples”, linked to a previous interface titled “Request Detail Page”, as indicated by the back-navigation breadcrumb. The interface includes a status indicator labeled “Partially Booked”, suggesting that the request has not yet been fully fulfilled. A first expandable panel labeled “Requester Details” presents metadata associated with the individual who submitted the biospecimen request. This includes fields such as the full name (“Robert Carter”), user ID, department, company affiliation (“Glenmark Pharma”), campus location, contact information (email address, office and mobile numbers), and the delivery address of the facility receiving the biospecimens (e.g., “9333 Genesee Ave Suite 170, Quest, San Diego, California, 92121”).

A second section labeled “Project Details” includes a job-specific identifier (“Project for HD Test codes July 2”), a job number (e.g., “SH-00000182”), the number of volunteers needed (2), a purchase order field, and the current delivery status. As shown, the delivery is indicated as “Partially Booked,” with the timestamp of the most recent booking (“July 9, 2025, 5:20 pm”). A third panel labeled “General Requirements” includes various screening criteria for donor eligibility, such as whether smokers are allowed (“No”), whether vitamins and herbal supplements are permitted (“Yes”), and whether all medications are allowed (“Yes”). Additional placeholders for listing excluded medications, donor inclusion criteria, and donor exclusion criteria are also provided.

A fourth section titled “Donor Selection” includes a dropdown interface to select a recruited donor, populated here with an example value “11Test Donors, Jul. 9, 2025 at 1:40 pm.” A punctuality interface allows the coordinator to log donor arrival behavior by selecting whether the donor was punctual (“Yes” or “No”), and if late, quantifying the tardiness in either minutes or hours. An additional “No Show” checkbox enables quick logging of missed appointments. This interface enables real-time tracking and management of specimen collection workflows, allowing administrative users to coordinate logistics, enforce eligibility requirements, and document compliance and participation. The GUI is implemented in a modular and scalable manner, supporting integration with external laboratory systems and logistics platforms, thereby facilitating operational efficiency in donor-based biomedical research environments.

### Role of an Administrator

An administrator within the Donor X platform has access to a comprehensive suite of controls enabling full operational oversight of biospecimen request workflows, donor engagement, and system configuration. Administrators can view all sample requests submitted by scientists, including draft, active, and completed jobs, and can access detailed metadata for each job such as project identifiers, sample types, donor counts, delivery timelines, and collection logistics. The administrator manages both the scientist and donor user populations. This includes onboarding new users, assigning user roles and permissions, and monitoring platform activity to ensure compliance with organizational protocols. Administrators may also be responsible for manual or semi-automated collection confirmation workflows, including donor check-in, label printing, sample status updates, and incident notation (e.g., delayed arrivals or collection complications).

In addition to managing routine sample requests, administrators have authority to process special requests, such as longitudinal studies, disease-specific recruitment, or matched multi-sample workflows. They can add and configure collection devices and sample types within the system, and associate these with predefined compensation values and appointment durations based on organizational or marketplace standards. Collection locations can also be added and updated by the administrator. For on-campus implementations, this includes internal labs and mobile units. For marketplace implementations, this includes external partners such as national laboratory chains, hospitals, or at-home phlebotomy providers. Admins input and manage address information, geolocation data, and site-specific scheduling rules.

Payment parameters can be defined for each biospecimen type, allowing administrators to differentiate compensation based on sample complexity, urgency, or donor condition. Messaging workflows, including automated email and SMS notifications, are also editable by administrators to reflect institutional branding, consent reminders, and logistics alerts. To support scalability, administrators can provision additional admin accounts and assign tiered permissions to distribute workload and enforce access controls. This delegation structure allows for modular administration by department, site, or functional role while preserving system integrity and auditability.

### Profile Management: Profile Settings

In some embodiments, the system provides administrative users with a profile management interface. Within the profile settings panel, administrators may update their mobile phone numbers for receiving SMS or push notifications related to job status updates and system alerts. System permissions restrict administrators from enrolling as donors, thereby ensuring separation of roles and compliance with confidentiality protocols.

### Admin Controls: Project Dashboard

The project dashboard available to administrators provides a centralized view of all biospecimen jobs, including those in draft, scheduled, or collected states. Each job entry includes metadata such as the expected delivery date, number of participating donors, sample types and quantities, pharmaceutical company name (in the marketplace instance), associated campus (applicable in both marketplace and on-campus systems), lab location, and the assigned scientist or requestor. A control menu, accessed through a three-dot interface, enables the administrator to view job details, initiate sample collection, cancel the job, or mark the job as picked up.

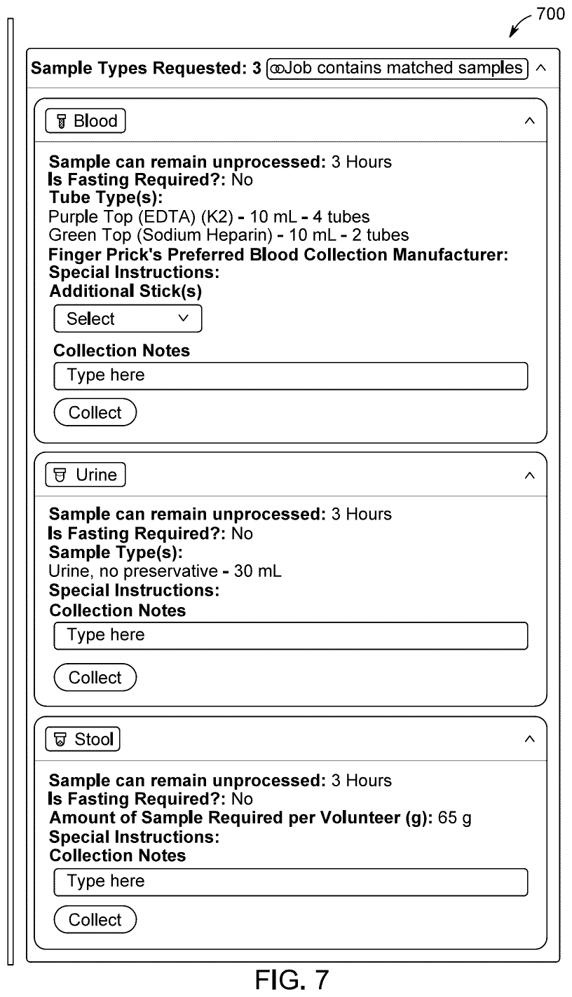

Referring to FIG. 7, the system provides a sample collection interface 700 configured to support the administration and recording of biospecimen collection activities. The interface is segmented by sample type and includes expandable and collapsible sections corresponding to blood, urine, and stool. Each section displays parameters relevant to the associated biospecimen.

For the blood sample block, the interface displays metadata such as the acceptable duration the sample can remain unprocessed (3 hours), whether fasting is required (No), and the tube types and quantities required. In the depicted example, the collection includes four 10 mL purple-top tubes containing EDTA (K2) and two 10 mL green-top tubes containing sodium heparin. The block also includes a field to specify a preferred finger-prick blood collection manufacturer, a dropdown menu for documenting the number of additional venipuncture attempts (“sticks”), a text entry field for special collection notes, and a “Collect”button to log completion of the blood draw.

For the urine sample block, the metadata includes the unprocessed time window (3 hours), fasting requirement (No), and sample specification (30 mL of urine with no preservative). A text field allows entry of special instructions or observations, and a “Collect”button logs the collection event.

For the stool sample block, the interface records the same 3-hour processing window and absence of fasting requirement, and specifies the required sample amount per volunteer (e.g., 65 grams), Similar to the other blocks, a text field and a “Collect” button are presented for entry of collection notes and confirmation.

The graphical layout enables administrative users or phlebotomists to document, track, and finalize the collection process for multiple sample types within a unified interface. Each section is individually expandable, allowing for streamlined data entry, visual feedback on matched sample types, and compliance with standardized biospecimen handling protocols.

### Admin Controls: Collect Samples

To initiate the collection process, the administrator selects the collect samples option from the control menu of a given job. This action directs the administrator to the job detail interface. For marketplace requests that do not automatically update donor status, the administrator must manually record the donation event. Within the job detail view, the administrator navigates to the donor selection block and selects the donor's real name from a dropdown list. This is distinct from the scientist-facing interface, which only displays de-identified donor IDs. After selecting the donor, the administrator indicates punctuality by selecting yes or noting the lateness.

Administrators may then print collection labels (a feature in development) and access specific collection instructions for the sample types involved. Once the collection is complete, the administrator selects the collected option to update the job status. Internal notes not visible to scientists may be entered at this stage. The interface also allows the administrator to document if multiple venipuncture attempts were needed; in the on-campus system, this may result in increased donor compensation.

Referring to FIG. &, the figure shows a screenshot of an administrative notification interface 800 that enables communication with the requestor following biospecimen collection The user interface comprises a dialog box labeled “Notify Requestor,” which provides the administrator with the option to initiate a notification event. The interface presents a binary selection—“Yes” or “No”—in response to the prompt “Would you like to notify the requestor that their samples are ready for pickup?”

Additionally, the system displays the number of completed donors relative to the total number expected (e.g., “Completed Donors: 0/2”), providing a visual reference for collection progress. Two actionable buttons labeled “Save” and “Exit” allow the administrator to either confirm and store the notification action or exit the module without saving. This interface ensures that communications regarding sample availability are handled in a timely and auditable manner, reducing the likelihood of miscommunication or missed pickups while enabling operational flexibility based on completion status.

### Notify Requestor

Within the on-campus platform, administrators may notify the scientist or requestor that samples are ready for pickup either after each collection or after all samples in a job are collected. This step is executed manually. In the marketplace configuration, however, this process is automated. When a courier service confirms pickup of the biospecimens, the system automatically notifies the scientist and provides a tracking link for real-time delivery monitoring.

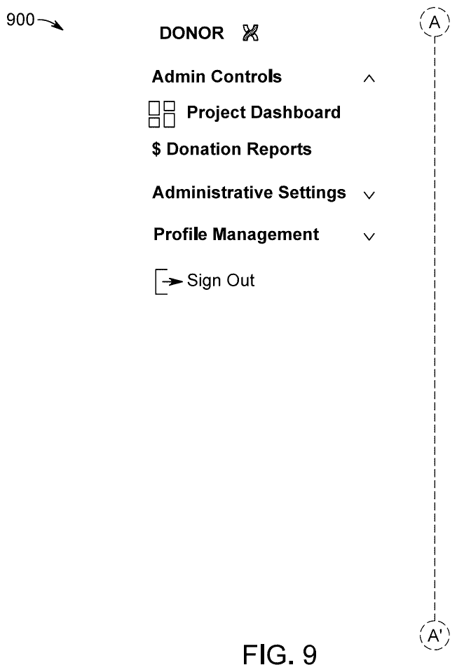

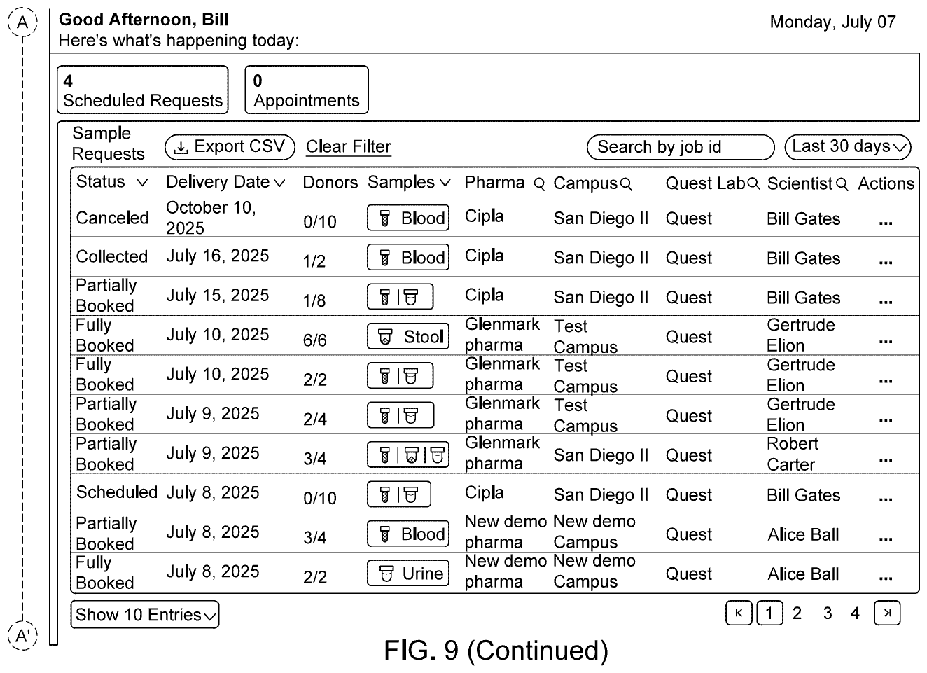

Referring to FIG. 9, the figure depicts a GUI 900 representing the administrative view of a project dashboard for biospecimen collection management. The interface displays a centralized summary of scheduled requests and appointments in prominent numeric tiles, which provide quick status awareness for the administrator. Below the summary tiles, a sortable and filterable table is presented, labeled “Sample Requests.”

Each row of the sample request table includes multiple attributes such as job status (e.g., Canceled, Collected, Partially Booked, Fully Booked, Scheduled), delivery date, donor fulfillment metrics, sample types (e.g., blood, urine, stool), pharmaceutical company or requesting organization, associated campus, designated lab for processing (e.g., Quest), and the name of the scientist linked to the job. The table is also equipped with an action column denoted by an ellipsis icon, enabling additional operations such as editing, viewing, or canceling the job. A filter panel above the table allows the administrator to export data in CSV format, clear applied filters, search by job identifier, or filter based on a time window (e.g., “Last 30 days”). The system includes pagination functionality at the bottom right, with navigation controls to access additional entries beyond the first page of results. This dashboard interface enhances transparency and operational control, providing administrators with a scalable and user-friendly way to manage multiple concurrent biospecimen projects across internal and external stakeholders.

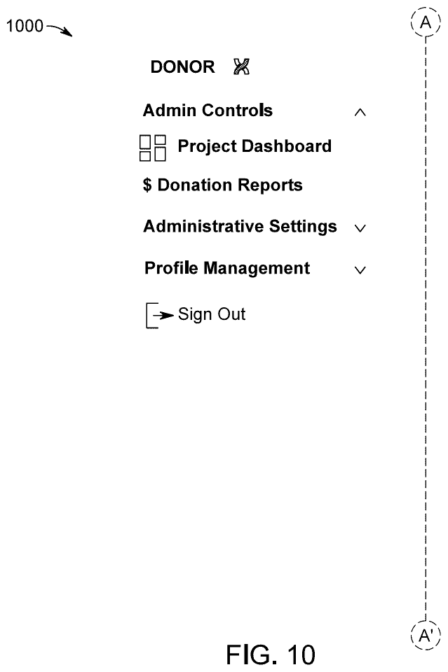

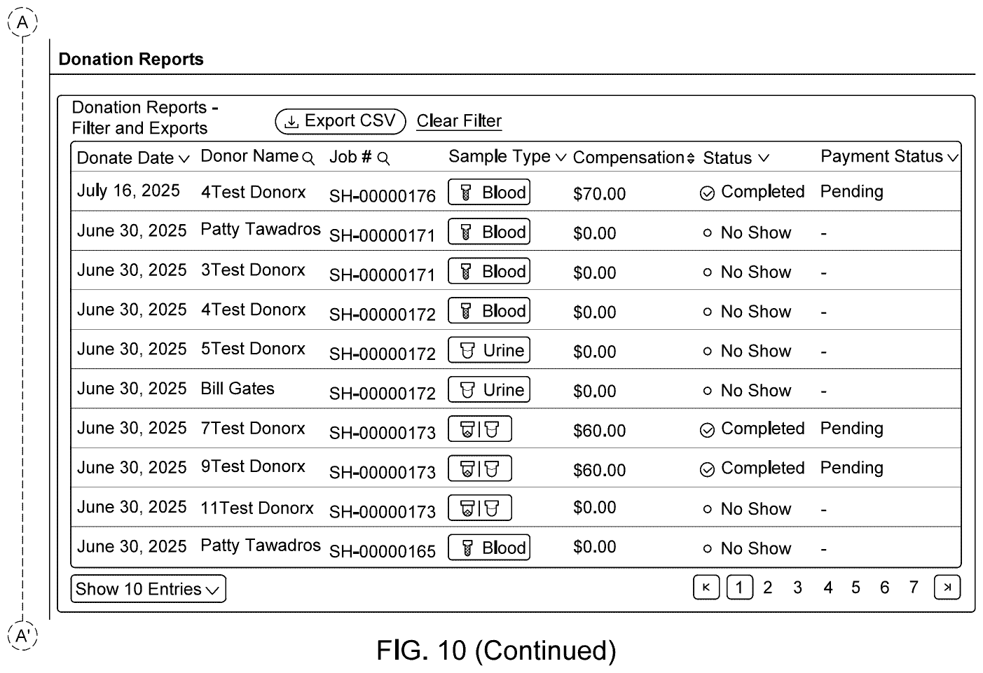

Referring to FIG. 10, the QUI 1000 depicts a Donation Reports dashboard configured for use by administrators. The interface enables access to a structured, tabular report of biospecimen donation events. The interface includes filtering and export capabilities to support administrative oversight and compliance tracking. The report displays key columns including donation date, donor name, job ID, sample type, compensation amount, attendance status (e.g., completed, no show), and payment status (e.g., pending, completed). Each row corresponds to an individual donation record. Sample types include blood, urine, and stool, indicated via distinct icons and color-coded labels. A compensation field captures the monetary amount associated with the donation, if applicable. Status indicators allow quick assessment of whether the donor completed their appointment, failed to attend, or is awaiting payment.

The user interface includes controls to filter data based on time range, donor name, or job ID. An “Export CSV” button is provided for generating offline reports. Pagination controls are displayed at the bottom of the report to navigate across multiple pages of historical donation data. This reporting module supports operational transparency and facilitates audit readiness, enabling administrators to track participation trends, donor engagement, and financial obligations. The design ensures that donation history can be easily reviewed and acted upon, whether for compliance, quality assurance, or logistics planning.

### Admin Controls: Donation Reports

In some embodiments, the system includes a donation reporting interface configured for use by administrative personnel. This interface enables administrators to access a consolidated record of all biospecimen collections for the purpose of initiating, tracking, and confirming donor payments. The donation reports interface may include sortable columns and filterable parameters such as donation date, donor name, sample type, compensation amount, job number, and payment status. In one configuration, administrators are provided with export functionality to generate comma-separated values (CSV) reports for internal tracking or submission to external payroll systems. In future implementations, integration to popular ERPs will be completed for automated reporting.

In further embodiments, the system supports an integrated automated payment module through a third-party financial data aggregator and payment facilitator. This module enables donors to securely link their bank accounts. Upon successful sample collection and verification, the system initiates a funds disbursement process. At the close of each business day, the payment engine automatically executes the transfer of compensation to the linked donor accounts. In the context of a marketplace deployment, administrators may access the same reporting interface to manually verify and export donation data to finance departments or external payment processors responsible for issuing compensation. The automated functionality may be implemented using secure APIs that handle identity verification, account authentication, transaction logging, and error reporting to ensure compliance, auditability, and traceability.

### Administrative Settings: Donor Management

In some embodiments, the system includes a donor management interface within the administrative settings module, enabling administrators to access, monitor, and manage donor profiles enrolled in the platform. Depending on the deployment configuration, such as an on-campus implementation or a public-facing marketplace, the donor population displayed to the administrator will vary. In the on-campus configuration, the administrator associated with a specific organization or institution may only view internal donors affiliated with that organization. In contrast, within the marketplace configuration, the administrator may access profiles of external, general-population donors registered through the broader platform.

The donor management interface allows the administrator to perform operations such as viewing individual donor profiles, initiating invitations to open biospecimen collection jobs, deactivating donors, or initiating payment actions. Donors may be directly invited to participate in a sample collection job by selecting the corresponding job listing, accessing additional actions via a dropdown or contextual menu, and selecting an “Invite” command. Donor records are sortable and filterable by various attributes including name, activation status, sample type preference, or enrollment date, and the dataset may be exported in a structured file format such as CSV for reporting or compliance purposes. The donor activation status is contingent upon completion of required regulatory documentation, including the signing of informed consent forms. Only upon proper execution of these forms will a donor be marked as “active” within either the on-campus or marketplace system configuration. This ensures compliance with ethical and legal standards for human biospecimen collection and handling.

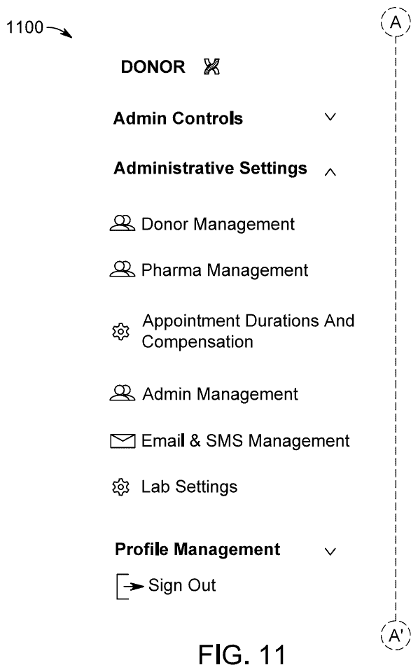

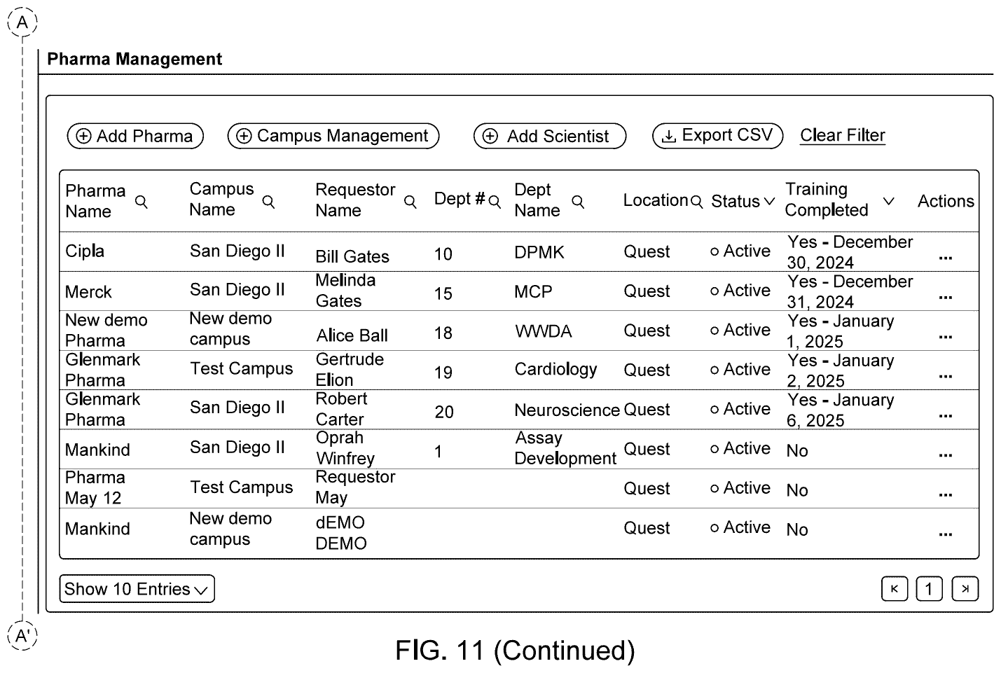

Referring to FIG. 11, the QUI 1100 provides an interface for managing pharmaceutical and research organization accounts (collectively referred to as “pharma profiles”) within the biospecimen fulfillment platform. This Pharma Management module enables administrators to view and control a list of registered organizations and their associated metadata. The left-side navigation menu includes links to Donor Management, Administrative Settings, Profile Management, and other system modules. The main panel contains a sortable table with columns displaying information for each pharma entity: Pharma Name, Campus Name, Requestor Name, Department Number, Department Name, Location, Status, Training Completion, and available Actions. The admin can initiate actions via buttons at the top of the interface, including “Add Pharma,” “Campus Management,” “Add Scientist,” and “Export CSV.” These actions facilitate the addition of new organizational profiles, campuses, or personnel, and allow for data export for auditing or backup purposes.

Each pharma entry in the table lists the associated scientist or requestor, the designated campus, department codes, collection site location (e.g., Quest), and account activity status (e.g., active or inactive). The training completion column reflects whether the associated personnel have completed system training and the corresponding date, which supports regulatory compliance and readiness tracking. The GUI 1100 streamlines the process of associating internal stakeholders to biospecimen projects and ensures that only trained and validated personnel are authorized to request and manage sample collection jobs. This system structure improves organizational transparency, enhances administrative oversight, and ensures scalable expansion across research institutions and pharma clients.

### Administrative Settings: Pharma Management

Within the administrative settings interface, the pharma management section enables authorized administrators to configure and oversee institutional and organizational entities that are eligible to initiate biospecimen collection requests. In an on-campus deployment, an administrator may create a new campus entry, designate a campus manager, and invite associated scientists or staff to enroll as approved requestors. Each campus entry can include identifying attributes such as the campus name, department affiliation, laboratory location, and authorized compensation policies.

In a marketplace deployment, the central platform administrator first creates a new pharma entity, then associates one or more campus entries with that entity, and subsequently invites individual scientists to participate under the designated campus. Each pharma or campus entry includes configurable payment terms, such as permissible collection volumes, price-per-sample rates, and accepted payment methods. If a credit card is selected as the preferred payment method, the system automatically activates secure payment functionality at the time of checkout, enabling requestors to fund their orders directly.

### Administrative Settings: Appointment Durations and Compensation

Within the on-campus software configuration, administrators define the default appointment duration and assign a corresponding compensation value for each sample type. Compensation may take the form of monetary payment or alternative, company. approved methods such as loyalty points or internal rewards. In contrast, within the marketplace configuration, appointment durations are predefined based on third-party laboratory capabilities and constraints. Marketplace administrators configure the dollar amount compensation for each sample type, with flexibility to implement dynamic pricing rules. These rules may include added bonuses for donors recalled for multiple appointments or differentiated compensation rates based on the presence of a documented disease state.

### Administrative Settings: Admin Management

This section provides a hierarchical access control structure wherein a super administrator may create and manage additional administrator accounts. The super administrator holds unrestricted permissions across the platform, while created administrators receive access based on configurable permission sets. These permission sets may grant access to system components such as Admin Controls, Project Dashboard, Donation Reports, Donor Management, and additional operational tools, allowing granular control over administrative responsibilities.

### Administrative Settings: Email and SMS Management

The communication interface enables platform administrators to manage all outbound email and SMS communications directed to donors, requestors, or fellow administrators. Message templates are accessible for both email and SMS and may be modified directly by selecting the message and editing its content. This provides flexibility in tailoring communications for appointment confirmations, sample collection reminders, compensation notices, and system alerts.

### Administrative Settings: Lab Settings

In the lab management section, administrators may configure laboratory details used for sample collection. For on-campus deployments, labs are tied to individual campuses and internally operated. In the marketplace configuration, laboratories are typically external facilities not owned or managed by the platform operator. The administrator inputs relevant lab information, including lab name, address, and other location identifiers. This information is incorporated into donor notifications, which include a hyperlink to navigation assistance using either the physical address or embedded GPS coordinates, enabling donors to locate their designated lab site efficiently.

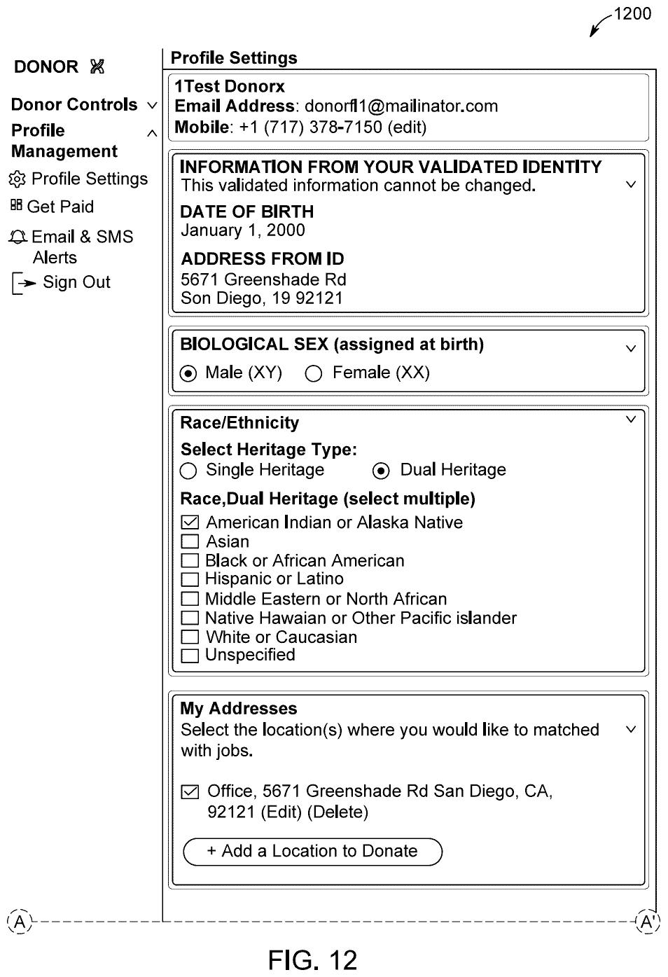

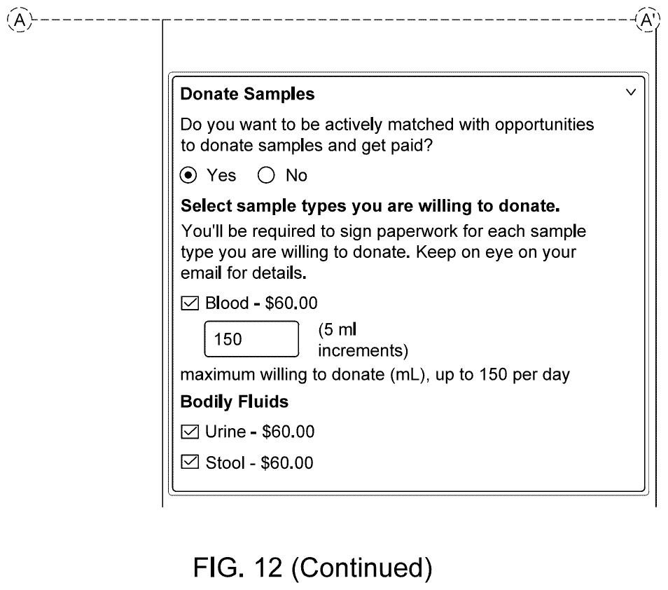

FIG. 12 illustrates an example profile settings interface 1200 presented within the donor platform. In some embodiments, the profile settings panel allows a donor to manage personal information, address details, and donation preferences, which are used by the system's matching engine to generate job recommendations. Verified identity information such as date of birth and government-issued address is displayed in a non-editable block, ensuring consistency with previously validated identification.

Below the identity block, the user may self-report biological sex as assigned at birth, followed by optional input fields for race and ethnicity. Donors may select either single heritage or dual heritage, with multiple race/ethnic background options available via checkboxes. These data points may be used in aggregate to support diversity tracking and filtering in studies where inclusion criteria require demographic representation. Address management enables donors to specify one or more geographic locations for donation eligibility. Addresses may be edited or removed and are used by the matching engine to determine logistical feasibility for job assignments. The final portion of the settings panel allows donors to opt into active matching and specify the types of biological samples they are willing to donate, such as blood, urine, and stool. Each sample type may be associated with a compensation amount and adjustable donation volume, as shown by the blood donation volume input control (e.g., 150 mL in 25 mL increments). This structured donor preference data enables automated eligibility filtering and supports dynamic job matching across both marketplace and on-campus configurations.

### The Role of a Donor

In the Donor X platform, each donor is assigned a unique, system-generated de-identified Donor ID composed of alphanumeric characters. This identifier is not visible to the donor, and is used solely for internal matching and privacy purposes. Donors interact with the platform through a secure profile management interface that allows them to maintain and update personal and health-related data. This includes demographic attributes such as age and race, lifestyle indicators such as alcohol or tobacco use, and responses to a structured health questionnaire. Donors may grant permission to link external health data sources, and may be asked to verify their health status through diagnostic testing, including phlebotomy-based blood tests. Within their profile, donors also specify the biospecimen types they are willing to provide, such as blood, saliva, stool, skin tape, or other materials. Donors review and execute digital consent forms associated with sample collection, and designate their preferred payment method from among supported options (e.g., Plaid, Venmo, PayPal). They also define logistical constraints such as geographic travel radius and appointment availability windows, enabling precise eligibility screening for job matching.

To ensure donor safety, especially for blood-derived samples, the system continuously monitors each donor's rolling blood donation volume against a predefined safe threshold (e.g., 500 mL per 8-week period for healthy donors). Donors are prompted at each login to confirm whether they have had blood drawn since their last session. If the response is affirmative, the donor is asked to identify the type of draw (e.g., routine medical lab test, blood bank donation, or plasma donation), the volume collected (if known), and the date. The system then calculates a dynamic eligibility score and restricts blood-related opportunities accordingly. Donors may also receive at-home biospecimen collection kits depending on their selected sample types. These kits may be returned via mail or delivered in person to a designated lab or shipping partner, enabling participation without the need for on-site visits.

Each donor has access to a personalized dashboard that includes historical data for previously completed jobs and an active job board displaying real-time opportunities for which the donor qualifies. The platform supports sorting, filtering, and job detail inspection within the dashboard. Donors who have a diagnosed disease or medical condition of interest may opt to set preferred compensation levels for specific biospecimen types. This allows for demand-sensitive pricing when their medical profile meets certain research criteria, and promotes donor agency in compensation negotiation.

### Donor Experience

To maintain the security of the platform and ensure compliance with research protocols, all users registering as donors must complete an identity verification process. Donor X uses a third-party service such as Plaid to authenticate donor identity based on full name, the last four digits of the social security number, and responses to dynamic security questions. Upon successful identity verification, the user account is activated and protected by modern authentication protocols, including two-factor authentication and one-time password (OTP) delivery to a verified mobile number.

### Informed Consent

Once identity verification is complete, the donor receives a digital informed consent form via email. This form is completed using a certified e-signature platform. The consent form must be reviewed and approved by an administrator, who may address any donor questions before granting full access to the Donor X platform.

### Profile Management: Profile Settings

The Profile Settings page displays the donor's name, email address, and phone number. Donors must provide a mobile number to enable 2FA login. Below this, the interface presents validated identity data imported from the verification provider, such as date of birth and address.

The donor then selects their biological sex (assigned at birth). Future platform versions will permit additional selections for gender identity and will allow donors to indicate hormone therapy or gender-confirmation surgery status.

Donors select their racial and ethnic background through a dual heritage selector, allowing either single or multiple selections from predefined categories such as American Indian or Alaska Native, Asian, Black or African American, Hispanic or Latino, and others.

### Donation Address Preferences

Donors may input multiple physical addresses where they are available to donate (e.g., work, school, home). Each address may be paired with a configurable radius to define a geographic match zone. Future iterations will allow donors to specify days of the week they are available at each location.

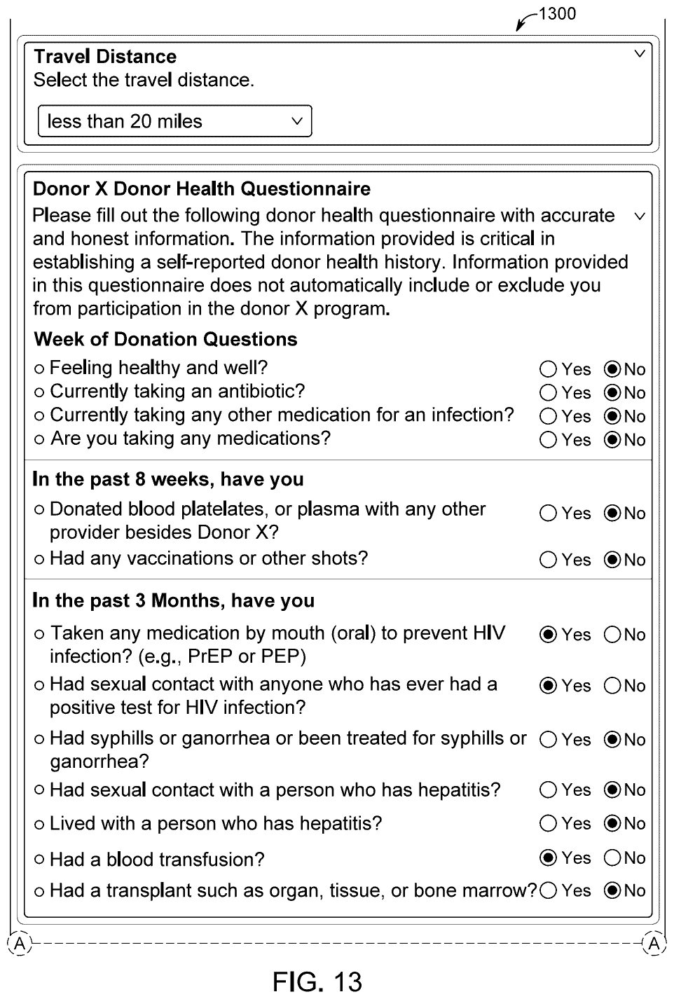

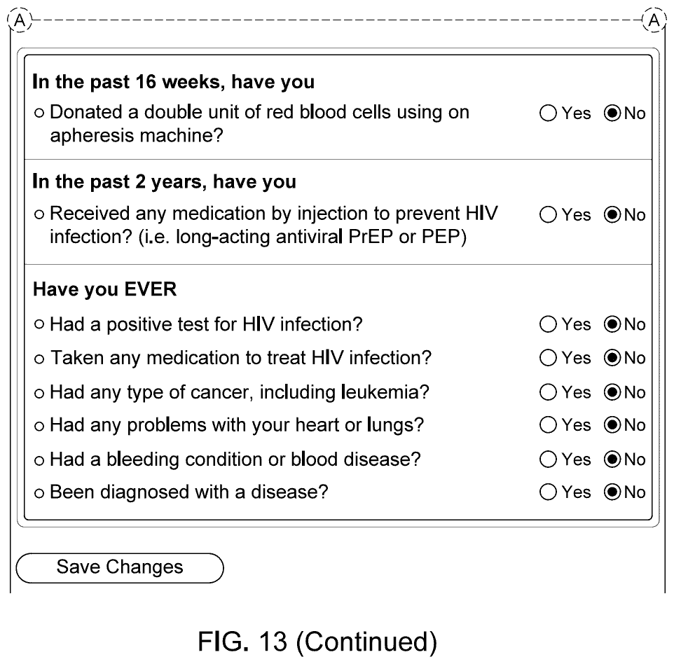

FIG. 13 illustrates an example donor health interface 1300 within the donor platform, which includes a travel distance selection module and a structured Donor Health Questionnaire. The interface is designed to support compliance with regulatory guidelines and institutional screening protocols for biological specimen donation. At the top of the interface, a travel distance selector enables a donor to indicate their preferred range of travel for donation appointments, allowing the system to filter location-based job matches accordingly.

Beneath this, the Donor X Donor Health Questionnaire enables dynamic entry of self-reported health information that may influence donation eligibility. The questionnaire is divided into temporal categories that inquire about recent health behaviors and exposures, including week-of-donation questions, recent vaccinations, infectious disease exposure, and medical treatments. Specific attention is given to factors such as HIV prevention/treatment medications, sexually transmitted infections, hepatitis exposure, and blood transfusion history. These questions are essential for automated eligibility scoring and real-time risk assessment.

The structured yes/no response format allows the platform to parse donor input using algorithmic logic to enable or disable certain sample type offerings, restrict specific jobs, or flag records for manual review. Information provided in this interface does not automatically disqualify a donor but is stored securely to ensure safety and traceability within the biological materials procurement pipeline. The system may also issue reminders for re-screening after a predefined time period, enhancing operational compliance and data integrity for both on-campus and marketplace models.

### Donate Samples

Within the Donate Samples section, donors can toggle whether they are actively matched for donation opportunities. They may opt in or out of specific sample types, such as blood, urine, or stool, and define limits on sample volume (e.g., maximum milliliters per day). Each sample type displays compensation information for transparency.

### Health Questionnaire

The donor health profile is maintained through a structured self-reported health questionnaire. The questionnaire captures data such as medical diagnoses, medication history, lifestyle factors, and other health-related variables relevant to eligibility criteria for sample-matching. Although this version is self-reported, future versions will allow lab test integration, document upload, and access to longitudinal health data from clinical lab systems or electronic health records (EHRs).

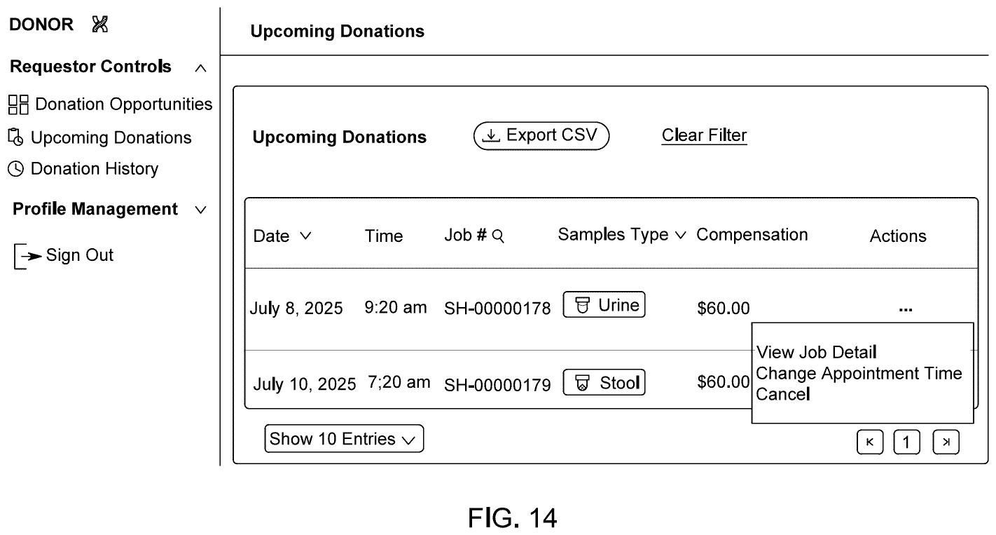

FIG. 14 illustrates an example graphical user interface 1400 for managing upcoming donations within the donor platform. The interface is accessed by a registered donor through a secure profile management system and is displayed upon selecting the “Upcoming Donations”tab from the left-side navigation menu.

The interface comprises a table listing one or more scheduled donation appointments. Each row of the table corresponds to a specific appointment and includes a plurality of data fields such as the donation date, time, job number, sample type, and compensation amount. In the example shown, two donations are scheduled: one for Jul. 8, 2025 at 9:20 am and another for Jul. 10, 2025 at 7:20 am. Each appointment includes a job identifier (e.g., SH-00000178 and SH-00000179), a sample type (e.g., urine or stool), and an associated compensation value (e.g., $60.00). The sample type is visually distinguished using a color-coded tag to improve legibility and reduce error during review. On the right-hand side of each row, an ellipsis icon is provided which, when selected, displays a contextual dropdown menu. This menu presents the donor with actionable options including “View Job Details,” “Change Appointment Time,” and “Cancel.” These options enable the donor to interactively manage their upcoming appointments through the user interface without needing to contact support staff, thereby improving efficiency and donor autonomy.

Above the table, user interface controls include an “Export CSV” button allowing donors to export a list of their upcoming appointments in a spreadsheet-compatible format, and a “Clear Filter” button to reset any applied filters to the default view. A dropdown menu labeled “Show Entries” allows for pagination by adjusting the number of displayed appointments on a single page. The interface is optimized for both desktop and mobile environments and is integrated with backend systems for real-time synchronization of appointment data, cancellation tracking, and rescheduling workflows. The functionality illustrated in FIG. 14 improves donor engagement, facilitates self-service appointment management, and supports operational scalability in donor scheduling systems.

### Donor Controls: Upcoming Donations

Within the donor platform, the Upcoming Donations screen provides each donor with a consolidated view of all accepted donation jobs. For each scheduled appointment, the interface displays key details including the date, time, job number, sample type (e.g., urine, blood, stool), associated compensation, location, and other relevant job-specific information. Donors are able to sort the entries in this view by selecting any of the column headers, such as Date, Time, Job Number, Sample Type, or Compensation. This functionality allows for easy navigation and prioritization based on the donor's availability or preference. Each donation entry includes an actions menu, accessible via a three-dot icon located in the “Actions” column. When selected, this menu presents the donor with options to view the detailed job information, change their appointment time, or cancel the scheduled appointment. While not depicted in the current user interface, a notification inbox is under development. This future feature will enable donors to receive and view system messages, job requests, and other alerts directly within the platform.

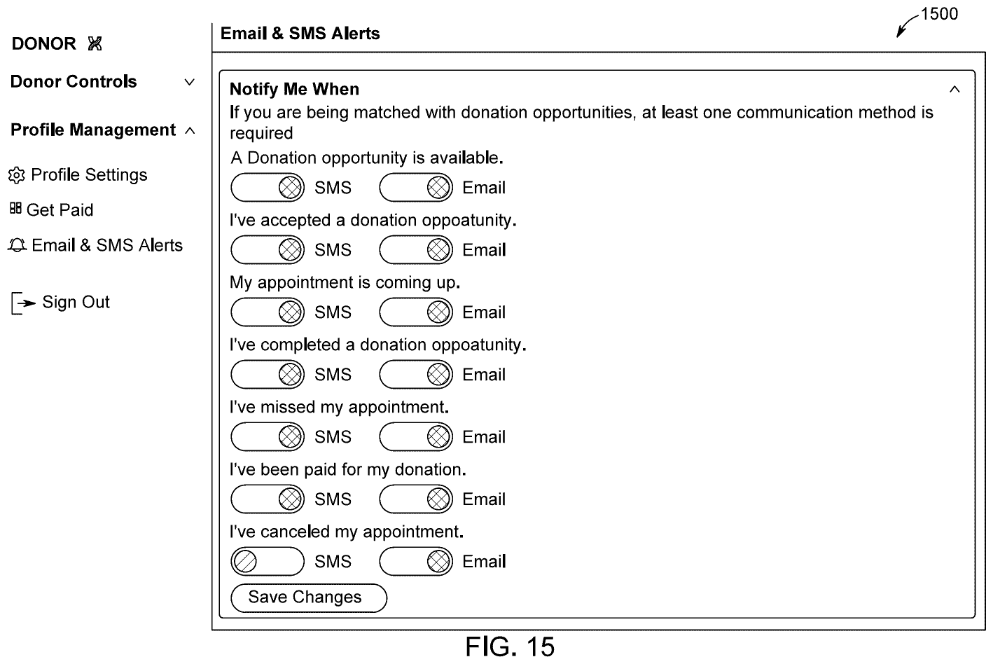

Referring to FIG. 15, donor interface 1500 presents a notification management module configured to allow a donor to select communication preferences for multiple types of donation-related events. The interface comprises a user-configurable panel with toggle inputs to activate or deactivate SMS and email notifications. The available events include, but are not limited to, when a donation opportunity becomes available, when a donation opportunity has been accepted, when an upcoming appointment is approaching, when a donation has been completed, when an appointment has been missed, when payment has been issued for a donation, and when an appointment has been canceled. For each event, the donor may independently toggle SMS and email notifications on or off, with at least one method required to be active for system compliance. The system stores the donor's preferences and transmits automated alerts based on these selections using backend messaging services integrated with the donor's registered contact information. This configurable interface improves donor engagement, reduces missed appointments, and enhances transparency in donation logistics by offering timely, user-directed communication.

### Profile Management: Email & SMS Alerts

Within the donor platform, the Email & SMS Alerts section allows donors to configure communication preferences for various donation-related events. Donors may opt-in to receive SMS notifications and may also select email as an alternative or additional method of contact. For each system-generated event, such as the availability of a donation opportunity, appointment reminders, completion of a donation, missed appointments, payment confirmations, and cancellations, the donor can individually specify whether they wish to receive notifications via SMS, email, or both. At least one communication method must be selected to ensure timely delivery of critical updates. This functionality provides granular control over how donors are engaged, allowing for an optimized and user-preferred communication experience.

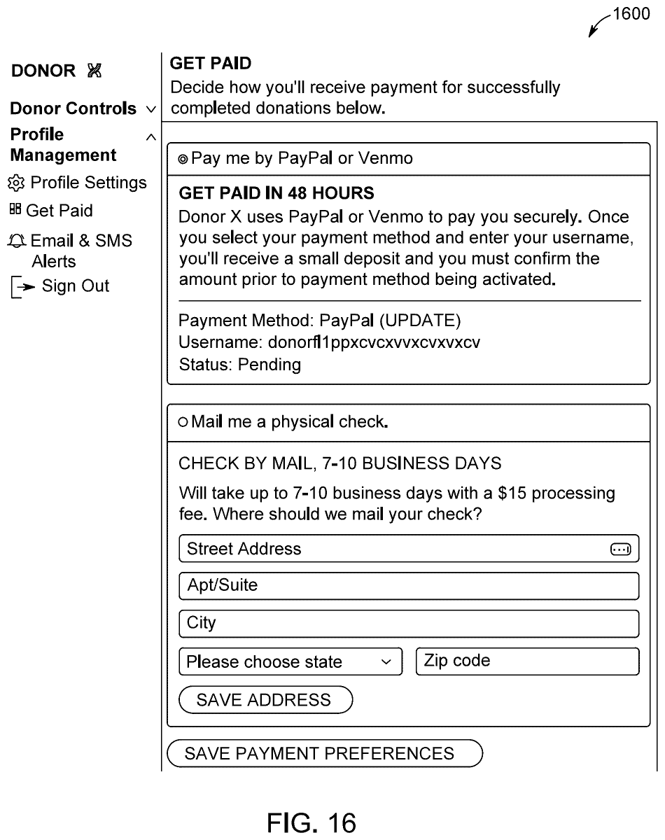

Referring to FIG. 16, interface 1600 illustrates an example donor compensation configuration interface that forms part of a graphical user interface (GUD) presented to a registered donor user of a biological specimen collection and management platform. Interface 1600 is accessible through the “Get Paid” module, which is invoked upon successful login and authentication of the donor through the platform's donor-facing dashboard.

The interface 1600 enables the donor to configure a payment method for receiving monetary compensation associated with previously completed and verified biological sample donations. The interface includes logic to detect and present selectable compensation options, including a first option for electronic disbursement via a third-party payment network and a second option for disbursement via a physical check transmitted through postal mail.

Upon selection of the first option, the system activates a data entry module that prompts the donor to input a payment network identifier (e.g., a username or handle associated with the electronic payment account). The interface is programmatically linked to a backend payment verification engine configured to initiate a micro-deposit of a predefined value (e.g., $0.01-$0.99) to the entered account. The donor is then required to verify the deposited amount by accessing their account and inputting the amount in a follow-up interface window. Only upon successful verification will the system associate the electronic payment account with the donor profile and activate the electronic disbursement method. Until such verification occurs, the status of the selected payment method is displayed as “Pending.”

If the donor instead selects the second payment option for physical checks, the system activates a mailing address capture interface configured to collect structured data inputs including street address, apartment or suite identifier, city, state (selected from a dropdown menu), and ZIP code. The interface may enforce formatting and validation routines to ensure data completeness and delivery eligibility. The interface further displays an informational prompt indicating that check disbursement may require 7 to 10 business days for processing and delivery, and may incur an additional transaction fee (e.g., $15). The system stores donor preferences upon activation of an interface element labeled “SAVE PAYMENT PREFERENCES.” Additional save logic is also implemented within the “SAVE ADDRESS” element, enabling separate storage of postal mailing address data for use in logistics, audit trails, or disbursement exception handling.

From a technical standpoint, interface 1600 improves donor workflow efficiency by enabling dynamic switching between disbursement methods, integrating asynchronous payment verification logic, and minimizing administrative overhead through user-directed configuration. Additionally, by modularizing the address input components and associating payment types with conditional UI activation, the system enables improved scalability across payment channels and geographic territories while maintaining robust compliance with user authentication, financial transaction verification, and privacy standards.

### Profile Management—Get Paid

The “Get Paid” interface within the Profile Management module enables donors to configure and manage their preferred compensation disbursement method for successfully completed biological sample donations. This interface allows donors to directly link a personal financial account using an integrated financial data API, such as Plaid or a similar secure banking authentication service, thereby facilitating real-time, direct-to-bank payments.

Alternatively, donors may select digital wallet options including PayPal, Venmo, or other third-party mobile payment platforms. The system is configured to authenticate the user-provided payment credential, such as a username or wallet ID, by initiating a verification sequence. In some embodiments, a micro-deposit is initiated to the designated payment account, and the donor must verify receipt of the deposit by submitting the precise transaction amount through the platform. Only upon successful verification will the selected payment channel be activated for use.

For donors opting not to use digital payment methods, the interface further provides an option to receive physical check disbursements via standard mail. This includes address input fields for street address, apartment or suite, city, state, and postal code, with appropriate formatting and validation routines. A notification is displayed to inform users of associated processing delays and administrative fees applicable to mailed checks.

The interface allows users to toggle between different payment options and persist selected preferences using a “Save Payment Preferences” action, which triggers backend logic to update the donor's profile record with the validated payment method and routing details. This modular configuration enables seamless financial disbursement workflows while preserving data security and regulatory compliance across jurisdictions.

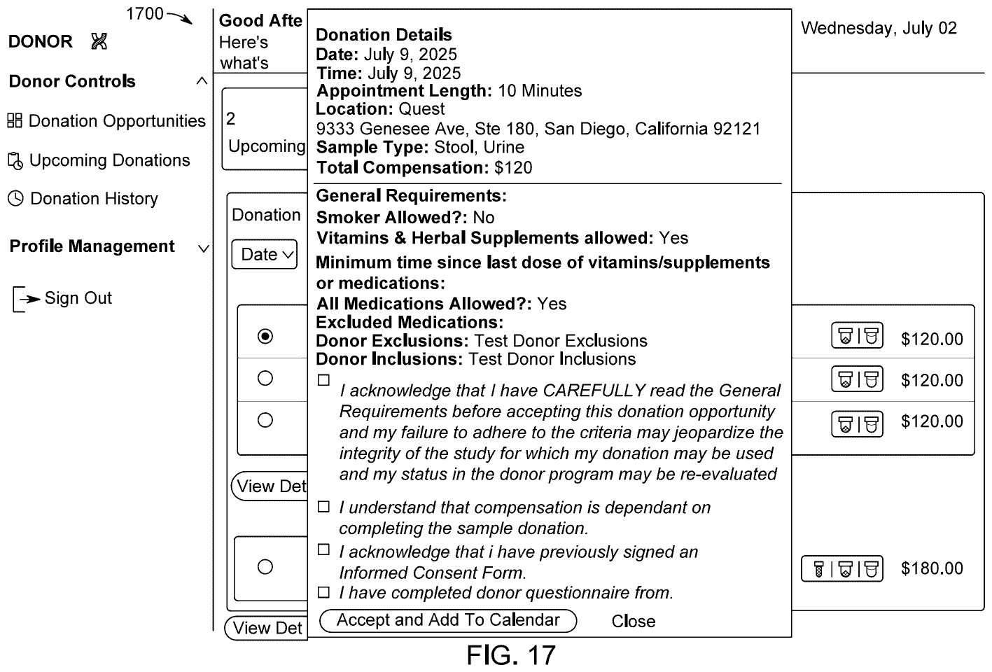

FIG. 17 illustrates a graphical user interface 1700 that is rendered within the donor-facing portion of the platform to display comprehensive information regarding an available biospecimen collection opportunity. The interface includes a structured layout that presents the date and time of the appointment, the total estimated duration for the collection, and the collection location, which may be a third-party laboratory such as Quest Diagnostics, including the full address. Additionally, the sample type requested for the job (e.g., stool and urine) is displayed along with the total compensation to be awarded upon successful completion of the donation.

Below the primary job details, interface 1700 displays a list of general requirements associated with the opportunity. These requirements may include conditions such as whether smokers are allowed, whether vitamins or herbal supplements are permitted, the minimum time that must have elapsed since the donor's last use of such supplements or medications, whether all medications are permitted, any medications that are specifically excluded, and textual identifiers for donor exclusion or inclusion criteria associated with the job.

Interface 1700 further includes a series of user-selectable checkboxes that the donor must affirmatively acknowledge prior to accepting the job. These checkboxes indicate that the donor has read and understood the general requirements, acknowledges that compensation is dependent on successful sample donation, confirms that an informed consent form has been previously executed, and verifies that a donor health questionnaire has been completed. Upon selection of the required acknowledgments, the donor may interact with an action control labeled “Accept and Add To Calendar,” which confirms their participation and integrates the appointment with their personal scheduling system. The interface supports verification logic to ensure mandatory confirmations are completed before submission and may include back-end validation through the Donor X platform database.

### Donor Controls: Donation Opportunities Dashboard

The Donation Opportunities Dashboard interface displays a filtered listing of biospecimen collection assignments for which the donor has been algorithmically matched based on pre-validated eligibility parameters. Each entry within the dashboard corresponds to a discrete donation opportunity and includes structured metadata such as job identifier, scheduled date and time, designated collection site location, requested biospecimen type(s), and total donor compensation. The donor-facing interface provides dynamic sorting functionality that enables the user to reorder job entries based on selectable attributes, including scheduled date, sample type, compensation amount, and geographic location.

Upon identifying an available opportunity of interest, the donor may initiate a detailed view operation by selecting a desired time slot and activating the “View Details” control. This triggers the rendering of a modal interface window populated with the job's predefined eligibility criteria and procedural requirements, including behavioral restrictions (e.g., medication and supplement usage) and donor inclusion/exclusion criteria. The system enforces compliance validation by requiring the donor to affirmatively check off each acknowledgement Geld, which includes confirmation of informed consent, review of job-specific requirements, and prior completion of a donor screening questionnaire. Once all required acknowledgments are selected, the donor can complete the reservation workflow by selecting the “Accept and Add To Calendar” control, thereby committing to the biospecimen collection event and triggering calendar integration. Alternatively, the donor may dismiss the modal interface by selecting “Close,”which exits the job preview without confirming participation.

### Donor Controls: Donation History

The Donation History interface provides a comprehensive, longitudinal record of all biospecimen donation jobs accepted by the donor. For each completed or pending engagement, the interface displays structured metadata including job identifier, scheduled date and time, associated biospecimen type(s), compensation amount, attendance status (e.g., punctual, no-show), and payment status. This interface functions as a centralized dashboard for donors to review their full engagement history within the Donor X platform.

The interface further includes a real-time blood donation volume tracker, which calculates and displays the cumulative volume of whole blood donated within a rolling 8-week interval. The system enforces a safety threshold of 500 milliliters over this interval for healthy donors.

An additional feature under development includes a login-triggered modal interface prompting the donor to self-report any external phlebotomy procedures that have occurred since their last login session. If the donor affirms such an occurrence, the system renders a structured form requesting the type of draw (e.g., clinical bloodwork, whole blood donation, plasma donation), the volume donated (if applicable), and the date of the draw. If the donor selects “doctor's visit” as the type, the system will apply a predefined estimate (e.g., standard diagnostic draw plus 50 ml, buffer) to update the donor's cumulative blood volume. Donors with disclosed medical conditions, such as diabetes or cancer, will be subject to an adjusted, more conservative threshold for allowable donation volume.

The Donation History page also allows donors to update their sample type preferences. This is achieved via the “Change Sample Types” control, which redirects the donor to the relevant section of their profile management interface for real-time preference editing.

### Sign out

The system supports both manual and automatic sign-out mechanisms. Donors may actively terminate their session via the “Sign Out” control, or they will be automatically logged out after a predetermined period of user inactivity in accordance with session timeout policies.

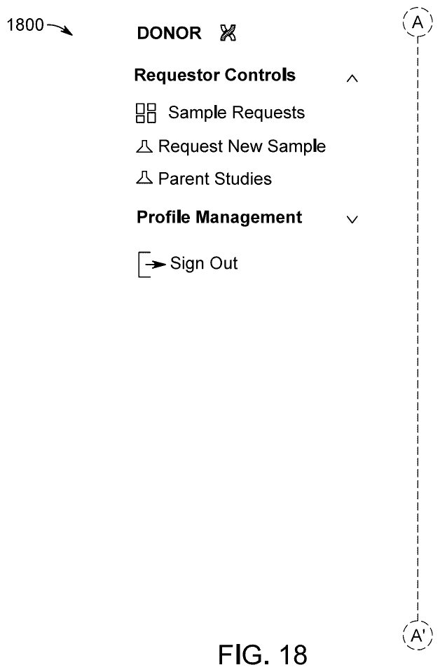

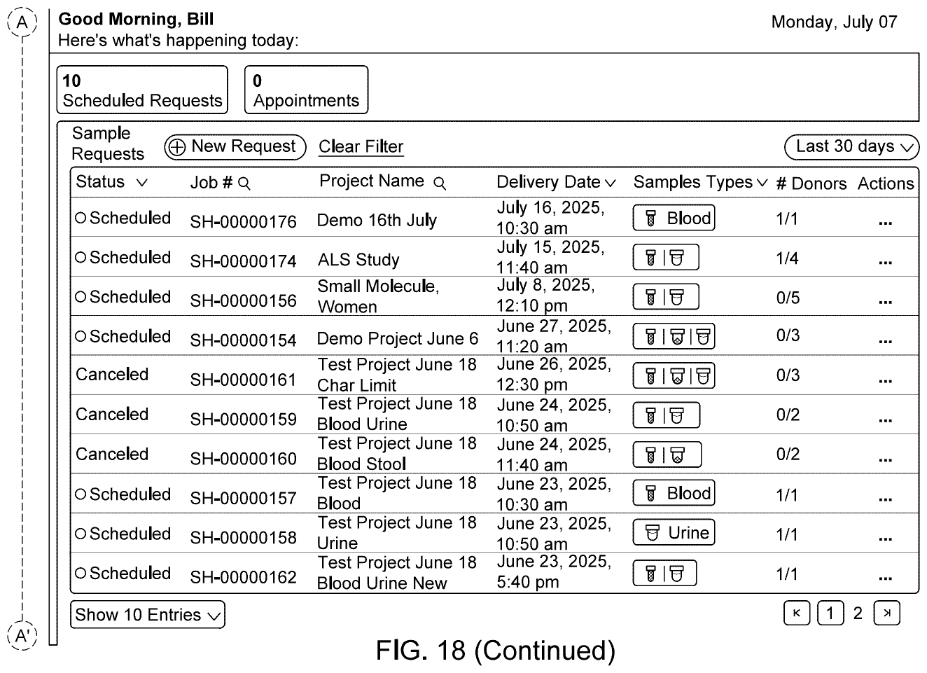

Referring to FIG. 18, user interface 1800 is an example dashboard displayed to a user operating in the role of a scientist or research requester within the Donor X platform. The dashboard interface is accessible via the “Sample Requests” tab under the Requestor Controls menu. The main dashboard provides a tabular overview of active and historical biospecimen requests initiated by the user, offering interactive sorting and filtering functionality. The upper portion of the interface includes visual summary counters displaying the total number of scheduled requests and upcoming appointments. These counters are dynamically updated based on system data.

The central sample request table contains multiple columns corresponding to individual request attributes. The “Status” column reflects the current lifecycle state of the request (e.g., Scheduled, Canceled), the “Job #” column displays a unique alphanumeric identifier associated with the request, and the “Project Name” field links each request to a designated study. The “Delivery Date” column shows the expected delivery timestamp for the collected samples, and the “Sample Types” column visually indicates the types of biospecimens required using labeled icons (e.g., blood, urine, stool). The “# Donors” column shows donor recruitment progress by indicating the number of donors matched to the total required (e.g., ¼). The “Actions” column, represented by an ellipsis icon, permits access to request-specific controls, including editing, duplication, cancellation, or exporting request data.

The scientist platform interface 1800 enables scientists to monitor the status of their sample recruitment efforts in real time and provides transparency into donor engagement metrics, request fulfillment, and delivery scheduling, Scientists may also create new requests using the “New Request” button, which triggers a multi-step input workflow to define sample specifications, inclusion criteria, and delivery constraints, All data on this interface are backed by system-generated updates originating from the Donor X scheduling, collection, and logistics engines.

### The Role of a Scientist

Scientists within the Donor X platform are provided with a dedicated portal to manage their profile, submit biospecimen requests, and track engagement with de-identified donors. Each scientist profile includes contact information such as institutional address, email address, and mobile phone number to enable SMS alerts and communication preferences. The profile also encompasses metadata such as affiliated organization, parent study identifiers, and researcher-specific donor filters including favorites lists, donor exclusions, and disease-specific wish lists.

Scientists are granted access to a dashboard that reflects all current and historical sample requests initiated through their account. For each sample request, the scientist can duplicate existing requests, cancel open requests, and initiate a recall for specific donors who have previously participated in prior jobs. Upon receipt of a biospecimen, the scientist may provide a sample quality rating and additional feedback pertaining to the donor's participation. The platform generates donor ratings both per specimen type and as an aggregate, which can be accessed by other approved scientists and used to refine donor selection criteria.

Following donor participation in a scientist's sample request, the system provides access to the de-identified health history and demographic profile of the matched donor. The full request and job history is stored within the scientist's portal, enabling exportable reports suitable for electronic lab notebook integration, grant documentation, or compliance audits. Participating donor data is accessible in a sortable dashboard view that allows filtering by demographics, disease state, donation date, and other attributes. Scientists can label or tag donors based on research classification (e.g., “healthy controls,” “type 2 diabetes,” “high-performing donors”) to streamline future donor recruitment workflows.

While the current version of the system does not support passive searching of the national donor registry, the next software iteration will enable scientists to search the donor population using attribute-based filters, such as age, race, smoking or alcohol use, medication history, diagnosis, zip code, and donor/sample performance ratings. Once identified, the scientist can tag desirable donor profiles to initiate customized recruitment campaigns.

To preserve donor privacy, no direct communication occurs between scientists and donors. Donor names, phone numbers, and email addresses are not disclosed. If a scientist wishes to recall a donor or issue a special request, the system triggers an administrative workflow. A Donor X admin is responsible for reaching out to the donor via approved communication methods and verifying their availability and willingness to participate. If the donor accepts, the appointment is scheduled, and the sample collection is initiated using any supported method—lab visit, in-home collection, shipping of a test kit, or mobile phlebotomy. The confirmed job details are updated in the scientist's portal, and the job progresses through the standard collection and fulfillment pipeline.

Referring to FIG. 18, user interface 1800 is an example dashboard displayed to a user operating in the role of a scientist or research requester within the Donor X platform. The dashboard interface is accessible via the “Sample Requests” tab under the Requestor Controls menu. The main dashboard provides a tabular overview of active and historical biospecimen requests initiated by the user, offering interactive sorting and filtering functionality. The upper portion of the interface includes visual summary counters displaying the total number of scheduled requests and upcoming appointments. These counters are dynamically updated based on system data.

The central sample request table contains multiple columns corresponding to individual request attributes. The “Status” column reflects the current lifecycle state of the request (e.g., Scheduled, Canceled), the “Job #” column displays a unique alphanumeric identifier associated with the request, and the “Project Name” field links each request to a designated study. The “Delivery Date” column shows the expected delivery timestamp for the collected samples, and the “Sample Types” column visually indicates the types of biospecimens required using labeled icons (e.g., blood, urine, stool). The “# Donors” column shows donor recruitment progress by indicating the number of donors matched to the total required (e.g., ¼). The “Actions” column, represented by an ellipsis icon, permits access to request-specific controls, including editing, duplication, cancellation, or exporting request data.

The scientist platform interface 1800 enables scientists to monitor the status of their sample recruitment efforts in real time and provides transparency into donor engagement metrics, request fulfillment, and delivery scheduling. Scientists may also create new requests using the “New Request” button, which triggers a multi-step input workflow to define sample specifications, inclusion criteria, and delivery constraints, All data on this interface are backed by system-generated updates originating from the Donor X scheduling, collection, and logistics engines.

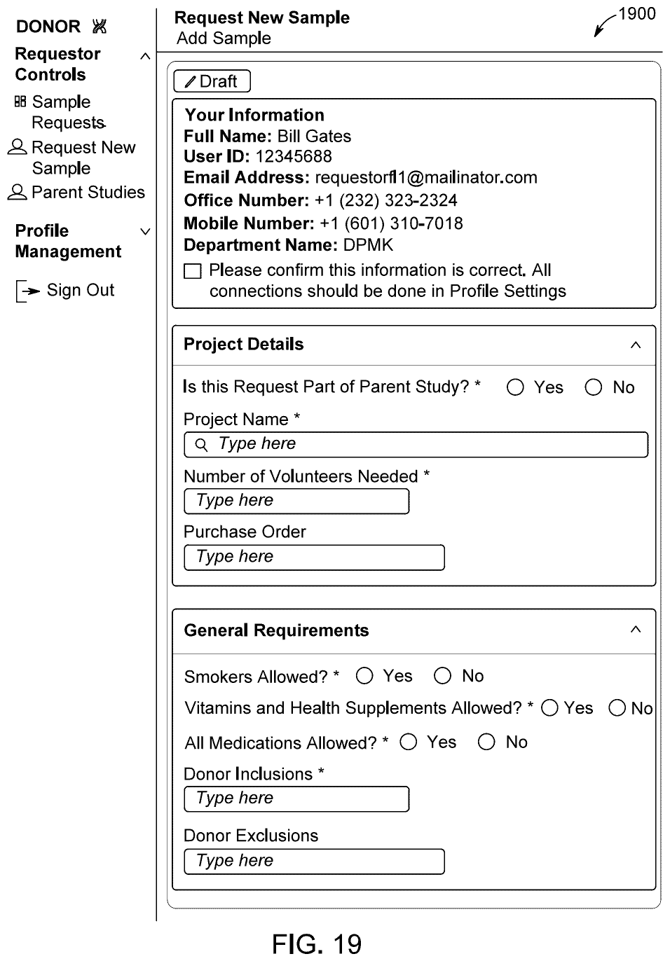

FIG. 19 illustrates an example interface 1900 presented to a scientist or requestor for initiating a new sample collection request within the biospecimen procurement system. The interface displays a section labeled “Your Information” which includes requestor-specific metadata such as full name, user ID, email address, office number, mobile number, and department name. The user is prompted to confirm or update this information through their profile settings. Below the identity section, the interface includes a “Project Details” module, where the scientist specifies whether the request is associated with a parent study, inputs the project name, the number of volunteers required, and optionally a purchase order reference. This data is used to correlate requests with institutional studies and financial tracking.

Further down, a “General Requirements” section allows the scientist to configure the biological and health-based inclusion and exclusion criteria for donors. This includes binary selections for whether smokers, vitamin and herbal supplement users, or individuals on medications are allowed. Additionally, free-text fields labeled “Donor Inclusions” and “Donor Exclusions” allow detailed entry of medical, behavioral, or demographic criteria that govern donor eligibility for the requested study. The configuration data entered in interface 1900 is used by the backend matching engine to recruit appropriate donors, trigger compliance checks, and schedule biospecimen collection logistics.

### Request New Sample Interface

The Request New Sample interface allows scientists to initiate a formal biospecimen collection request. The initial portion of the form presents the scientist's contact information, including name, email, phone number(s), and department. The scientist is required to validate the accuracy of this contact information by checking a confirmation box. This verification step ensures proper routing of samples and adherence to internal protocols regarding sample matching and delivery logistics.

### Project Details

The Project Details section enables the scientist to indicate whether the current request is associated with a Parent Study. If the request is linked to a Parent Study, the platform may support workflows involving repeat donors who previously contributed to that study. The scientist is further prompted to enter the project name, the number of volunteers needed for the request, and any applicable purchase order numbers for internal tracking and billing.

### General Requirements

This section allows the scientist to define basic eligibility criteria for donors. These include binary selections for smoker status, use of vitamins or herbal supplements, and medication allowances. Additionally, the scientist can input detailed inclusion and exclusion criteria into free-text fields. The current configuration does not support dynamic filtering based on longitudinal health data or specific disease states, but such capabilities may be enhanced in future iterations.

### Sample Request

In this portion of the form, the scientist specifies the biospecimen parameters, such as sample types (e.g., blood, saliva), collection tube types, and required processing instructions. The interface also supports input fields for donor-specific targeting, including the ability to recall particular donors or exclude certain individuals. Once fully implemented, this section will support list-based controls, allowing scientists to invite or exclude donors from curated favorites, tagged groups, or exclusion lists.

### Acknowledgement

Prior to finalizing the request, the scientist must review and acknowledge any conditions regarding sample quality, handling, and timing. This step may include a requirement to input initials or digitally sign the request to confirm agreement. Once complete, the scientist can choose to either select “Schedule Now” to proceed to the scheduling module or “Save for Later” to preserve the request as a draft for future completion.

### Schedule

Upon clicking “Schedule Now,” the scientist is directed to the calendar module, where they can browse available collection windows and select a preferred delivery date and time. This calendar-based scheduling supports standard workflows but does not accommodate complex special requests. For such cases, where specific donors must be contacted and availability aligned, the platform supports a separate workflow. In that process, an administrator coordinates directly with the donor, confirms availability, and sets a custom appointment time and location. This may include options for at-home test kits or collection by a mobile phlebotomist. Once the special appointment is confirmed, the job is updated in the system, and the scientist receives a notification including the scheduled delivery date and donor participation details.

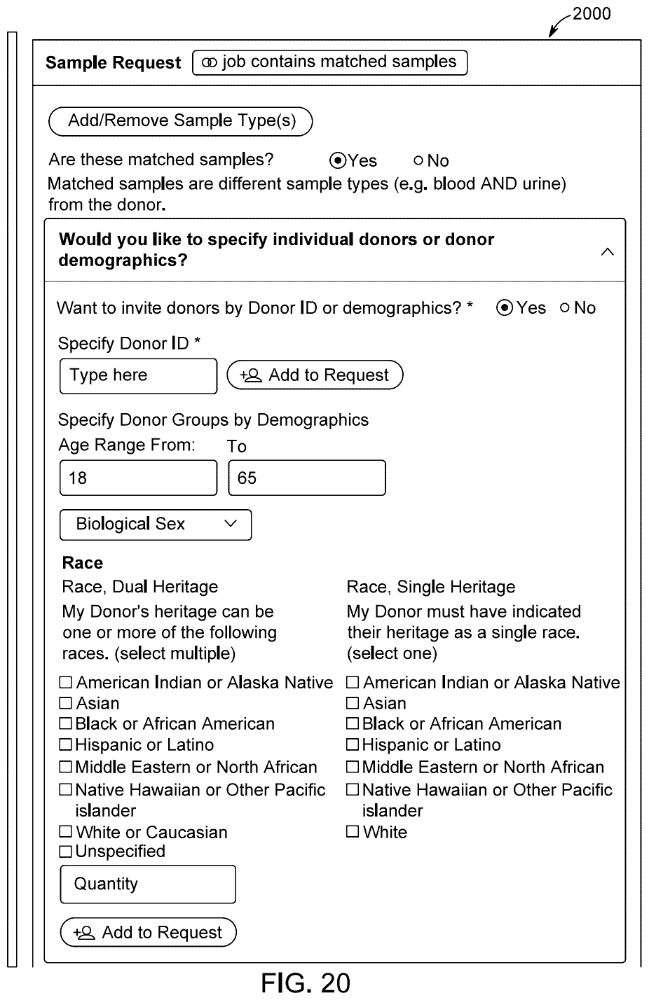

FIG. 20 shows an interface 2000 of a sample request configuration screen used by a scientist to define specimen matching criteria and select specific donors or donor populations based on customizable parameters. The top section includes a toggle field labeled “Job contains matched samples” and an interface control for adding or removing sample types. Matched samples refer to the simultaneous collection of different specimen types (e.g., blood and urine) from the same donor. Below this, the interface prompts the user to specify whether donors should be selected by individual Donor ID or by broader demographic criteria. If the user selects “Yes” to invite donors by Donor ID or demographics, two fields become active. The first field allows manual entry of one or more Donor IDs with an option to add the identified donor to the request. The second field enables demographic filtering by selecting a donor age range (e.g., 18 to 65), biological sex, and race.

The interface offers two parallel racial heritage categories: one for donors identifying with multiple racial groups (“Race, Dual Heritage”) and another for single heritage identification (“Race, Single Heritage”). The scientist can select one or more racial categories under each type. Examples include Asian, Black or African American, White, Hispanic or Latino, Native Hawaiian or Other Pacific Islander, and others. A quantity field is included to designate the number of donors desired that match the specified criteria. Once all relevant fields are populated, the user may select the “Add to Request” button, which will update the scientist's job requisition and initiate the donor recruitment workflow in the backend system based on the supplied demographic and specimen requirements.

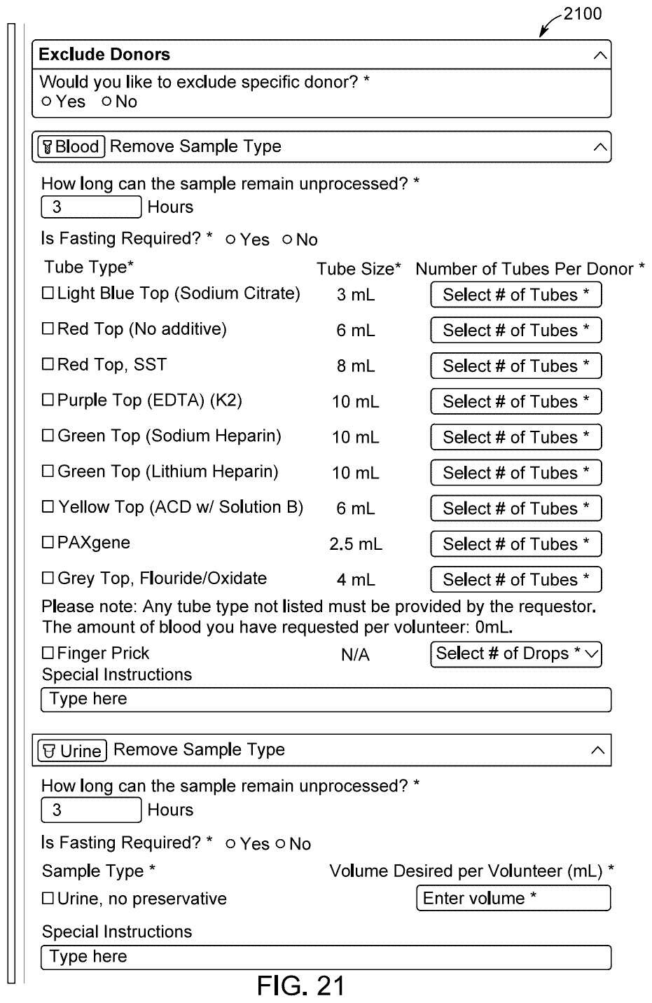

FIG. 21 depicts a user interface screen 2100 used by a scientist or requester to specify biospecimen collection preferences and restrictions. The interface begins with an “Exclude Donors” section, prompting the user to indicate whether a specific donor should be excluded from the request by selecting either “Yes” or “No.” Below this section, the interface contains a configurable module for the collection of blood samples. The sample type is identified as “Blood” with an option to remove the sample type if necessary. The user may define how long the blood sample may remain unprocessed, such as three hours, as well as whether fasting is required prior to collection.

The interface further provides a comprehensive listing of available blood tube types with associated tube sizes. The selectable options include Light Blue Top (Sodium Citrate) at 3 mL, Red Top (No additive) at 6 mL, Red Top SST at 8 mL, Purple Top (EDTA) [K2] at 10 mL, Green Top (Sodium Heparin) at 10 mL, Green Top (Lithium Heparin) at 10 ml., Yellow Top (ACD with Solution B) at 6 mL, PAXgene at 2.5 ml, and Gray Top (Fluoride/Oxalate) at 4 mL. For each selected tube type, the scientist must specify the number of tubes to be collected from each donor. The system automatically calculates the total amount of blood requested per volunteer based on these parameters. An additional option is provided for collecting blood via finger prick, allowing the user to specify the number of drops required. A free-text input field is available for entering special instructions related to the blood collection.

A separate module for urine collection appears below the blood collection section. Within this section, the scientist can specify how long the urine sample may remain unprocessed, whether fasting is required prior to collection, the desired sample type (such as urine without preservatives), and the target volume to be collected per volunteer in milliliters. A text field is also provided for entering any additional collection instructions specific to the urine sample. The interface depicted in FIG. 21 enables detailed configuration of sample collection protocols to ensure compliance with research requirements and safeguard the scientific utility and integrity of the specimens collected.

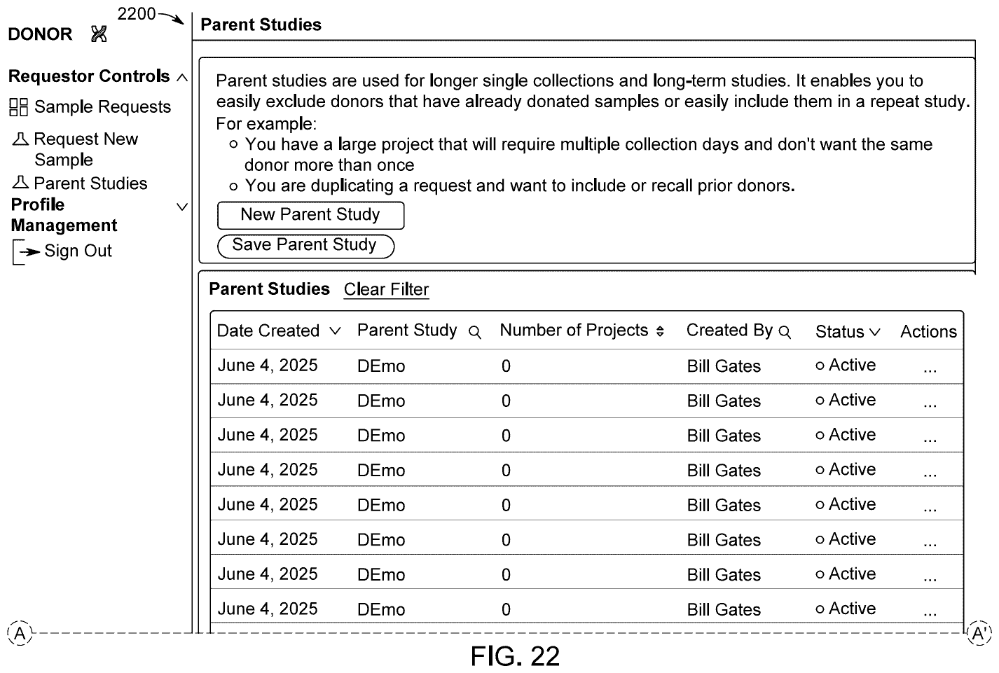

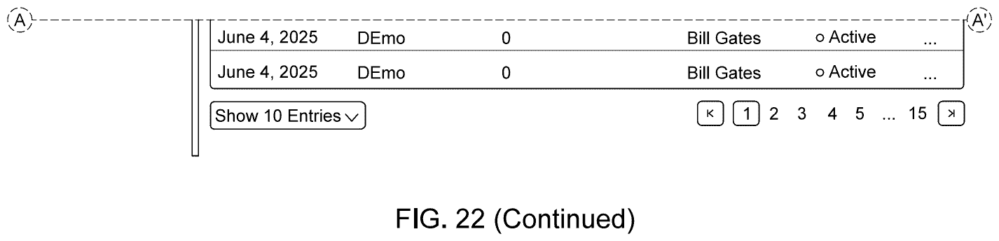

FIG. 22 shows a user interface screen 2200 enabling scientists or authorized users to manage parent studies within the biospecimen procurement platform. The parent study feature is intended for larger studies that require multiple collections or for repeat research where donor history and inclusion or exclusion decisions need to be tracked efficiently. At the top of the interface, explanatory text describes the utility of parent studies. It clarifies that this feature allows users to exclude previously participating donors or include specific donors from earlier jobs, particularly when conducting longitudinal studies or duplicating existing requests. A button labeled “New Parent Study” enables creation of a new parent study record, while a “Save Parent Study” button allows the user to finalize and store the study entry.

Below this, the interface displays a table listing existing parent studies. Each row of the table includes the date created, indicating when the parent study record was entered (e.g., June 4, 2025); the parent study name (e.g., “Demo”); the number of projects currently linked to that study; the name of the user who initiated the study (e.g., “Bill Gates”); the status, such as “Active,” to indicate that the parent study is ongoing; and an actions menu, which allows the user to access options for editing or managing the parent study. The list is paginated, with navigation controls allowing users to browse multiple pages of parent study records. This module facilitates traceability and streamlined donor management across multiple related biospecimen requests under a unified research objective.

### Parent Studies

The Parent Studies dashboard presents a centralized view of all parent studies associated with a scientist's account, including those created by the scientist and those to which the scientist has been granted collaborator access for sample requests. From this interface, the scientist may initiate the creation of a new Parent Study or manage existing studies by executing commands such as Edit, Pause, or Delete. The dashboard is sortable by column headers including the date the parent study was created, the study name, the total number of linked projects, the creator's name, and the current status (e.g., active or inactive). Additionally, the user may access and review individual projects grouped under each Parent Study, enabling coordinated management of longitudinal or multi-collection research initiatives.

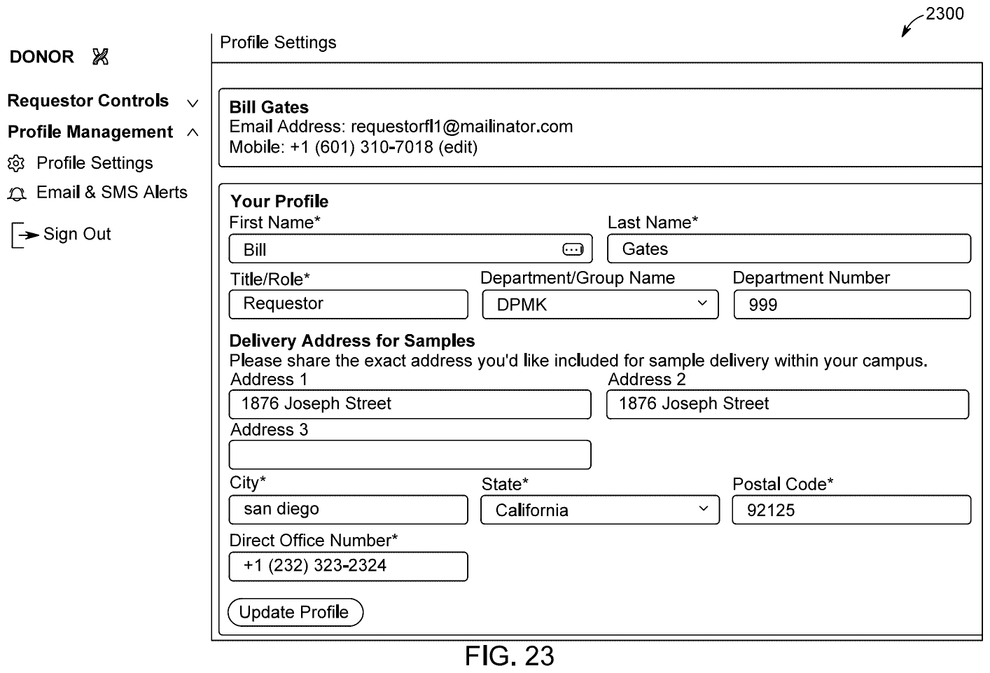

FIG. 23 shows an interface 2300 in which a user, such as a requestor or scientist, may manage their profile settings. At the top of the screen, the user's name (Bill Gates), email address (e.g., requestor #1@mailinator.com), and mobile number are displayed, with an option to edit the contact number. The “Your Profile” section includes editable input fields for the user's first and last name, title or role (e.g., “Requestor”), and department or group name (e.g., “DPMK”), along with a department number. This profile section ensures correct attribution of biospecimen requests and alignment with internal organization structures. Below, the “Delivery Address for Samples” section collects logistics information relevant to shipping and receiving biospecimens. The user may input one or more address lines, including street address, city, state, and postal code. Additionally, a direct office number is provided to facilitate courier communication or sample drop-off coordination. An “Update Profile” button allows users to save any changes made to the entered information. This configuration screen ensures that communication preferences and delivery logistics remain current and tailored to the operational needs of the requesting party.

### Profile Settings

Scientists have access to a dedicated profile management interface within the platform, which enables them to maintain and update essential account and logistical information. This includes editing their mobile number for SMS notifications, specifying their delivery address for biospecimen shipments, entering a direct office contact number, and updating professional identifiers such as title, group name, and group number. These fields ensure accurate communications and logistical coordination aligned with the scientist's affiliated institution or research entity.

### Email & SMS Alerts

Scientists may configure alert preferences through a communication settings panel. This functionality allows them to manage which types of alerts they wish to receive and to designate the preferred communication method, such as email, SMS, or both, for each category of notification (e.g., job confirmations, donor participation updates, or sample shipment tracking).

### Requestor Controls: Sample Requests

The primary dashboard interface for scientists is accessible under the “Sample Requests” section of the requestor controls. This dashboard provides a comprehensive view of all sample requests the scientist has submitted or initiated. Each entry includes key metadata such as job number, project name, current status (e.g., scheduled, draft, canceled), delivery date, requested sample types, and associated donor information. The dashboard includes features for sorting and filtering entries, and it supports export functionality to generate reports in Excel or CSV formats. Additional controls are embedded within each request entry via an action menu (represented by three dots). Through this menu, scientists may access the full job details, edit draft entries, cancel scheduled jobs, or duplicate existing requests for reuse. At the top of the dashboard interface, scientists are provided with a summary snapshot displaying the number of upcoming scheduled requests and associated appointments. A prominently positioned “New Request”button enables direct navigation to the sample request order form.

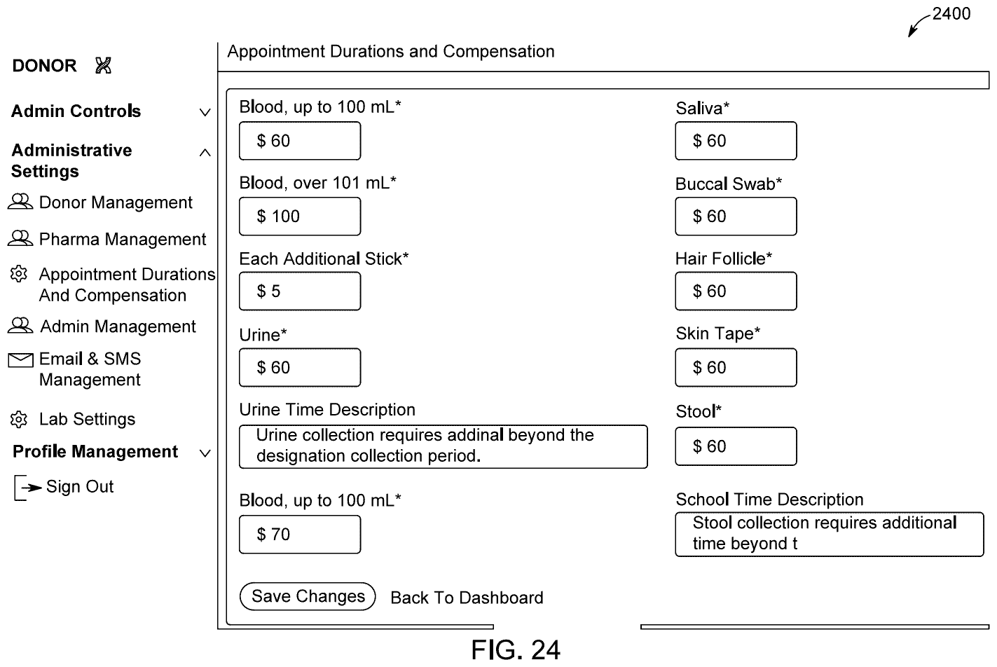

FIG. 24 presents a graphical user interface 2400 accessible through the “Appointment Durations and Compensation” section under administrative controls. This interface allows administrators to configure payment values associated with different biospecimen collection procedures. The screen is divided into multiple labeled fields corresponding to specimen types, each field accepting a monetary value representing donor compensation. For blood collections, different compensation tiers are shown based on volume, such as up to 100 mL (e.g., $60) or over 101 mL (e.g., $100). Additional blood draws via a secondary needle insertion (“Each Additional Stick”) may be compensated separately (e.g., $5). Compensation values are also assigned to other specimen types, including urine, exhaled breath (e.g., $70), saliva, buccal swabs, hair follicles, skin tape, and stool (e.g., $60 per collection for each).

Text boxes are provided to describe special timing or collection logistics that might impact compensation, such as additional time requirements for urine or stool sample collection. At the bottom of the screen, administrative users may save any modified compensation rates or return to the main dashboard. This administrative interface supports flexible, specimen-specific compensation configuration for research organizations or laboratories, ensuring transparency and operational consistency across various biospecimen collection types.

### List Management

List Management is a feature that is planned for future development within the scientist interface of the Donor X platform. This feature will allow scientists to create and manage categorized donor lists such as “Favorites,” “Exclude from All Studies,” “Healthy Donors,” “Diabetic Donors,” and other user-defined classifications. These lists will be referenced during the donor selection process when a scientist is completing a sample request order form, enabling targeted recruitment and streamlined donor matching based on past participation or specific health traits.

### Donor Dashboard

The Donor Dashboard provides scientists with a centralized view of all donors who have participated in their sample requests. The interface displays donor-related metadata including Donor ID, gender, race, and sample types provided. Scientists may export donor health histories and assign individual donors to lists created in the List Management module. This dashboard serves as an overview and control center for tracking donor engagement and characteristics across multiple studies.

### Payment

The Donor X platform supports multiple scientist payment options, depending on account setup during onboarding. Scientists may be authorized to pay via Purchase Order, Credit Card, or through integrated third-party research marketplaces such as Scientist.com or ScienceExchange.com. Pricing structures and payment terms are configured during the initial onboarding process and reflected in the scientist's account settings.

### Sign Out

Scientists may manually sign out of the platform at any time. Additionally, the system is configured to automatically log users out following a predetermined period of inactivity to protect account security and sensitive data.

### EXAMPLE 1

### On-Campus Biospecimen Collection Platform

In one embodiment, the biospecimen procurement platform is deployed in an on-campus configuration, enabling a research organization, such as a pharmaceutical company, university, hospital, or biotechnology firm, operate the system internally for the collection of biospecimens from its employees. In this implementation, both scientist users and donor users are affiliated with the same organization; however, the platform is configured to maintain strict de-identification between the roles. Donors are assigned system-generated donor IDs composed of alphanumeric strings, and these identifiers are stored in a secure database accessible only to a designated program administrator. The identity of each donor remains hidden from the requesting scientist throughout the lifecycle of the specimen job.

Access to the system is managed by the administrator, who invites authorized scientists to join the platform and submit biospecimen requests through a structured, authenticated interface. Simultaneously, the organization promotes the platform internally and invites employees to enroll as de-identified donors by completing a digital consent process and structured health profile. The administrator may be an internal employee of the organization or a third-party technician, such as a phlebotomist or external research coordinator, with administrative privileges.

When an organization licenses the on-campus platform, the software is configured to reflect institution-specific parameters. These include predefined biospecimen types (e.g., whole blood, saliva, urine, skin tape), associated collection protocols, approved collection locations, standard appointment durations, and internal donor compensation policies. The organization may also define collection methods (e.g., in-clinic draws, self-collection kits), shipping instructions, and institutional branding within the user interfaces. All of these variables can be updated by the administrator through a secure configuration module at any time.

Biospecimens are typically collected in designated on-campus laboratories or health units staffed by a company-employed phlebotomist, a contracted technician, or a service provider authorized by the platform vendor. Collection locations are configured with hours of operation, equipment capabilities, and geographic metadata for scheduling optimization. In some cases, at-home collection kits may also be issued by the organization, allowing donors to self-collect certain sample types and return them via internal drop-off points or prearranged courier pickup.

When a scientist submits a biospecimen request through the on-campus platform, the request is processed by the system's scheduling engine and transformed into one or more structured jobs. Each job includes a specific appointment time, collection location, biospecimen metadata, and logistical instructions. The system uses donor-matching logic to identify eligible participants within the organization's donor pool, applying demographic and health-based filters, appointment availability, and collection constraints. Qualified donors receive automated invitations that include the job details, compensation information, and the option to accept, decline, or reschedule. Upon acceptance, the appointment is reserved and removed from availability to other donors.

At the time of collection, donors check in to the assigned location, where their identity is verified and consent is re-confirmed. The system retrieves the relevant job record, and labels are generated containing the de-identified donor ID, job number, collection date and time, and sample type. These labels are printed on demand and affixed to each biospecimen container in accordance with institutional procedures. The collection is marked complete within the platform, triggering downstream updates. The requesting scientist is notified through their dashboard that the sample is collected and available for pickup, or, if configured, that it is being routed to another internal or external location. The administrator has visibility into all completed and scheduled collections, enabling oversight of specimen throughput, resource allocation, and billing. In the on-campus model, each department may be billed internally for collected specimens in a manner defined by the organization's finance team. The platform supports billing configuration to allocate costs per department, project code, or research unit.

The system includes built-in safeguards to prevent conflicts of interest or violations of data protection protocols. For example, a scientist can be both a requestor and a donor, so the platform checks for organizational hierarchy conflicts and automatically blocks jobs from being shown to a donor that works within the same group as the requestor, as that would violate this policy. In addition, the donor identity remains inaccessible to the scientist throughout the process, ensuring ethical boundaries and IRB compliance are upheld. Overall, the on-campus deployment mode provides a secure, scalable, and privacy-preserving method for research organizations to operate internal biospecimen collection programs. The platform enables streamlined coordination of specimen logistics, automated donor matching, de-identified sample labeling, and real-time communication across stakeholders, all while maintaining institutional control over compensation, compliance, and infrastructure.

### Audience

In one embodiment, the system comprises an on-campus biospecimen collection platform designed for use by research organizations such as pharmaceutical companies, universities, biotechnology firms, or individual researchers. The platform enables these entities to collect human biospecimens directly from their own employees. In this configuration, both the scientists (also referred to as requestors) and the donors are internal members of the same organization. However, donor identities are de-identified with respect to the scientists. Each donor is assigned a unique donor identifier, which is only accessible to the program administrator, thereby maintaining privacy and compliance with institutional and regulatory standards.

### Accessing the System

The platform is accessed via invitation by a program administrator, who may be an internal employee of the organization or a designated third-party administrator such as a contracted phlebotomist or a representative from Donor X. Scientists are invited to join the platform and are granted the ability to submit biospecimen requests. Simultaneously, employees across the organization are informed about the opportunity to participate as donors and are provided instructions on how to enroll. Upon enrolling, their identities are pseudonymized through assignment of a donor ID.

### Roles

The system recognizes three primary roles in the on-campus implementation: scientists (requestors), donors, and administrators. Scientists are internal researchers submitting biospecimen requests. Donors are internal employees who voluntarily participate by donating samples. Administrators are individuals responsible for managing the platform, configuring workflows, and ensuring the proper separation of donor and scientist identities. The administrator role may be performed by company personnel or outsourced to a third-party contractor, such as a phlebotomist or a service provider affiliated with Donor X.

### Customization

When a research organization acquires a license to deploy the on-campus platform, the software is tailored to meet the organization's operational needs. Customizable parameters include sample types (such as blood, saliva, urine, or skin tape), approved collection methods (such as in-clinic or at-home kits), laboratory locations within the organization's premises, appointment durations, and donor compensation rules. Additionally, the platform allows configuration of internal billing mechanisms. All such settings are controlled through the administrative interface and may be modified in real-time to reflect changes in organizational policy or project requirements.

### Collection Locations

Sample collections are conducted within a designated on-campus laboratory. The facility may be staffed by an internal phlebotomist employed by the organization, a contract medical professional, or personnel provided through a service agreement with Donor X. The organization may also authorize the distribution of at-home sample collection kits, at its discretion. These kits are prepared with appropriate labeling instructions and are returned via pre-approved delivery channels.

### Differences

The on-campus configuration differs significantly from the marketplace model. In the on-campus version, both the scientists and the donors are members of the same organization, and the entire biospecimen workflow is executed within the organization's internal infrastructure, Although sample collection is physically localized, the system permits shipping of biospecimens to external facilities if necessary. The compensation for sample donation is determined and administered by the organization, and employees are paid in accordance with internal policies, which may include cash equivalents, points, or other approved incentives. Organizational departments are billed for the samples collected in a manner consistent with the organization's financial procedures. To safeguard against potential conflicts of interest, the platform is designed with built-in safeguards that prevent scientists from placing sample requests that might result in the collection of their own biospecimens or those of their immediate collaborators. These controls are enforced through identity-blind request matching and audit logs maintained within the system. This architecture ensures operational integrity, donor anonymity, and alignment with ethical and regulatory requirements.

### EXAMPLE 2

### Marketplace Human Biospecimen Collection Platform

In another embodiment, the biospecimen procurement platform operates in a marketplace configuration, allowing scientists and research organizations to acquire human biospecimens directly from members of the general public through a distributed, cloud-based system. This implementation is optimized for decentralized specimen sourcing and leverages third-party collection infrastructure through secure system integrations and programmable APIs.

The marketplace model supports participation from pharmaceutical companies, universities, diagnostic firms, and independent researchers. Scientist users are typically employees of external organizations authorized to submit biospecimen requests. Donor users are unaffiliated members of the public who voluntarily register on the platform to be matched with specimen jobs. System administrators are employees or agents of the platform provider, responsible for managing users, approving special requests, coordinating with collection partners, and ensuring regulatory compliance.

Access to the platform is invitation-based and identity-controlled. Each scientist or donor must be invited to register and complete a multi-step identity verification process. Identity verification may include knowledge-based authentication, partial social security number validation, or a third-party digital identity tool configured to verify personal information against banking or medical records. Verified users receive role-specific access credentials and are presented with dashboards tailored to their participation type.

Unlike the on-campus configuration, the marketplace platform does not rely on proprietary brick-and-mortar infrastructure. Instead, it is integrated with external biospecimen collection networks, including national laboratory chains, hospital systems, outpatient clinics, mobile phlebotomy services, dialysis centers, and diagnostic imaging providers. These third-party facilities serve as authorized collection locations. The platform establishes direct data connections with partner systems through RESTful APIs or secure data exchange layers. These integrations allow the platform to read and write scheduling data, check appointment availability in real time, receive confirmation of completed collections, and optionally retrieve relevant donor lab data if consent is granted.

When a scientist submits a biospecimen request, the platform processes the request using internal job orchestration logic and performs a real-time query of third-party collection site calendars. Available appointment times and locations are returned to the scientist interface, where the user selects their preferred delivery timeframe. The system accounts for multiple dynamic factors when generating delivery options, including donor proximity to collection sites, expected sample processing time, delivery route estimates, and buffer time for courier dispatch.

To ensure operational reliability, the platform automatically overbooks each request by adding one or more backup donors to every job. For example, if a scientist requests two blood samples from males aged 40-60, the platform may match four donors who meet those criteria and stagger invitation timing to ensure redundancy. Backup logic is dynamically adjusted based on historical no-show data, sample type, and geographic collection patterns. This algorithmic overbooking strategy minimizes disruption due to donor cancellations or missed appointments.

In certain cases, donors may be eligible for at-home collection workflows. For minimally invasive sample types such as stool, skin tapes, nasal swabs, or saliva, the platform can dispatch a self-test kit to the donor's residence. The kit includes pre-labeled containers, printed instructions, a scannable code, and a QR code to be scanned at a drop off location. Once the sample is collected, the donor delivers the kit to an authorized shipping partner. At check-in, the shipping partner scans the code, affixes a machine-generated label containing the donor ID, job number, and collection timestamp, and dispatches the specimen for delivery,

Alternatively, for phlebotomy-based sample types, the platform can assign a mobile collector to visit the donor's home. The collector brings the appropriate containers and pre-printed labels, verifies donor identity at check-in, and performs the draw under protocol-compliant conditions. The collector then completes the chain-of-custody documentation and hands off the sample to a courier or drops it at a designated shipping location. In either case, collection data and label metadata are pushed automatically to the platform, which updates the job records across donor, scientist, and admin dashboards.

The platform supports dynamic integration of donor health history. With explicit consent, a donor may link their profile to a third-party provider, such as a diagnostic lab, health system, or personal health record service, to import longitudinal lab data. This imported data becomes part of the donor's profile and may be used to enhance matching accuracy or allow scientists to request samples from donors with recent diagnostic test results (e.g., hemoglobin A1C, CRP, or liver function panels). After the sample is collected, a completion record is generated by the integrated collection site or mobile collector. This record is ingested by the platform's backend services, which triggers downstream operations. The requesting scientist is notified that the sample is in transit or available for retrieval. Sample tracking metadata is made available in real time, including shipping provider status, delivery ETA, and confirmation of chain-of-custody compliance.

Donors are compensated following verified collection. The platform includes a payment module that supports a variety of disbursement methods, including direct bank transfers, digital wallets, or physical checks. Compensation values are defined at the job level and may vary depending on sample type, rarity of donor profile, collection complexity, or turnaround urgency. Once the system receives confirmation of collection, the donor's compensation is released via the preferred method. The platform logs all payment events and reconciles them against job records for auditing and tax reporting purposes.

### Audience

The marketplace model of the Donor X platform is designed for external research organizations, including pharmaceutical companies, academic institutions, biotechnology firms, and individual researchers, to obtain fresh human biospecimens directly from public donors. Scientists affiliated with these organizations place structured biospecimen orders through the system and receive matched specimens via a coordinated network of collection partners and shipping services.

### Accessing the System

Access to the Donor X system requires an invitation and verified identity. Scientists, donors, and administrative users (admins) must undergo authentication, including identity validation using tools such as Plaid or similar verification APIs that confirm identity via government-issued ID, contact details, and financial institutions. Donor X employees, acting as program administrators, are responsible for initiating invitations to both scientists and donors to onboard them into the platform environment.

### Roles

There are three primary roles within the platform: (i) scientists who are affiliated with organizations that submit biospecimen orders, (ii) donors who are members of the public providing samples, and (iii) admins who are employees of Donor X. Although the scientist identity and associated institution are not revealed to the donor during the ordering process, the platform allows for the possibility that a donor may coincidentally be employed by the requesting institution. Access controls and de-identification prevent conflicts of interest and preserve confidentiality.

### Collection Locations

Donor X does not operate its own physical collection facilities. Instead, it integrates with an ecosystem of third-party collection partners using secure software APIs. These partners include national diagnostic laboratory chains (e.g., Quest Diagnostics, LabCorp), hospital-affiliated labs, university clinical units, dialysis centers, physician networks, outpatient collection centers, and mobile phlebotomy services. By embedding API endpoints, the platform can scan availability, book appointments, transmit job details, retrieve lab test results, and confirm successful collections.

### Differences in Collection Integration

A distinguishing feature of the Donor X marketplace is its technical integration with existing biospecimen collection infrastructure. Using a combination of REST APIs, webhook-based notifications, and custom scheduling logic, Donor X's platform can: (1) query third-party calendars to identify available appointment windows, (ii) confirm bookings with real-time feedback, (iii) ingest donor screening results into structured records, and (iv) support identity-based longitudinal data linking when a donor provides consent. This enables continuous synchronization with clinical laboratory systems and automated delivery of results to scientists and administrators.

### At-Home Collection and Direct-to-Consumer Workflows

For minimally invasive samples such as skin tapes, fecal specimens, or buccal swabs, the platform supports direct-to-consumer shipping of self-collection kits. Each kit includes a scannable identifier. Donors transport completed kits to a designated shipping partner, whereupon system-generated labels are printed with key metadata: Donor ID, Job Number, Collection Date, and Time. Labels are affixed to both sample containers and outer shipping packages to ensure traceability. Alternatively, mobile phlebotomists may visit the donor's location to collect blood or other samples, affix labels in compliance with chain-of-custody requirements, and dispatch the shipment using authorized couriers. Both workflows are fully traceable, with updates pushed to the platform interfaces used by scientists, donors, and admins.

### Integrated Scheduling and Courier Coordination

The system dynamically calculates the logistics of each job using a scheduling algorithm that incorporates donor availability, lab hours, courier windows, and sample viability constraints. Delivery timeframes shown to scientists reflect not just collection time but also total expected time to delivery at the scientist's designated receiving location. Backup donors are automatically assigned to account for expected no-show rates. Initially, the platform applies a 2:1 donor-to-job ratio, which is modifiable as attendance patterns are learned by the system.

### System Integration with National Laboratory Chain

Donor X has finalized integration with a national clinical laboratory network comprising more than 2,000 service locations in the U.S. and select locations in Canada. Through direct API connection, the platform facilitates appointment selection, confirmation, sample collection logging, and automatic courier dispatch to fulfill biospecimen orders. Each interaction is logged and updated in the system, allowing full visibility into the biospecimen lifecycle.

### Real-Time Matching and Overbooking Logic

When a scientist places a biospecimen order, Donor X conducts a real-time search of connected lab calendars. The system retrieves eligible appointment slots and allows the scientist to select from among those results. To ensure fulfillment, the platform uses predictive overbooking logic to recruit alternate donors in anticipation of cancellations or missed appointments. This logic is designed to adapt as the platform collects real-world data on donor behavior.

### Donor Payment

Once a sample is successfully collected and verified, the system issues payment to the donor using the payment method specified in the donor's profile. Currently supported methods include direct bank deposit via one or more third party credit card processing vendors, and/or a physical check (with processing time and fee applied). The platform is modular and supports integration with additional payment providers as the ecosystem evolves.

This technical configuration transforms biospecimen acquisition into an automated, scalable, and compliant process, eliminating the need for bespoke recruitment efforts or internal lab infrastructure while ensuring high-quality, traceable, and ethically sourced human samples.

### EXAMPLES

### Rare Disease Donor Recruitment and Revenue Sharing Integration

In some embodiments, the disclosed system includes functionality for integrating third-party donor recruitment organizations specializing in rare diseases into the Donor X platform through a permissioned administrator account interface. These third-party entities, referred to as referral companies, may maintain proprietary databases of donors with rare, genetically confirmed, or otherwise medically verified conditions that are in high demand for biomedical research. Through a revenue-sharing agreement, such companies are enabled to contribute their donor cohorts into the Donor X system for use in scientific study recruitment. Each referral company is provisioned with administrative access credentials allowing them to log in to a secure portal of the Donor X platform. Within this portal, they may upload verified donor information to the centralized donor database. The uploaded records may include health history documentation, eligibility criteria, and diagnostic confirmation data, which are subject to validation workflows within the system. Upon upload, each donor record is tagged with a unique association to the originating referral company. This association is persistent and used to track participation, attribution, and financial distribution events.

When a scientist or sample requestor utilizes the platform's sample ordering interface and selects a donor tagged to a referral company, the system programmatically logs the donor-company relationship and triggers a referral credit. In response to fulfillment of the sample request and confirmation of donor participation, the Donor X platform executes a revenue-sharing protocol whereby a predefined portion of the transaction value is allocated to the associated referral company. The system is further configured to provide the referral company with non-identifying analytics and engagement metrics concerning their donor population. In some embodiments, the referral company may access a secure dashboard displaying the number of completed sample opportunities involving their referred donors. However, to preserve research confidentiality and prevent reverse identification of studies, the platform does not expose the identity of the scientist or organization initiating the request to the referral company. The rare disease recruitment module supports data integrity and compliance with healthcare privacy standards by maintaining access control, encryption of donor health history, and role-specific permissioning for referral company administrators. This implementation enables a scalable and ethically managed revenue-sharing model while increasing the availability of rare disease biospecimens for qualified research institutions.

### EXAMPLES

### Market Research Participant Recruitment System

In some embodiments, the system includes a market research integration module that enables third-party market research organizations to recruit eligible participants from among the registered donor population within the Donor X platform. This functionality is enabled through role-based access control and data segmentation protocols that preserve donor confidentiality while facilitating targeted outreach by external research entities. Market research companies are provisioned with limited-access administrator accounts configured to execute search operations across a subset of the donor database. These search operations are constrained to those donor profiles that have explicitly opted in to participate in market research initiatives. Opt-in status is stored as a structured data field within each donor's profile and is accessible only for accounts authorized for market research operations.

Upon logging into the system, authorized market research users are presented with a search interface that enables filtered queries based on donor metadata, including but not limited to age range, race, biological sex, gender identity, relevant health history attributes, and declared interests or behavioral characteristics. The system returns anonymized results matching the search criteria, with each donor represented by a unique market research identifier distinct from the clinical donor ID used for biological sampling purposes. This market research ID is used to track eligibility and engagement without revealing personally identifiable information (PII) at the initial stage. The platform further enables market research companies to generate curated candidate lists based on search results. These lists may be exported via structured data files or API endpoints for use in downstream participant management systems. Upon export, and only after the research organization has selected specific individuals for outreach, the system authorizes the release of donor contact details, including name, email address, and any additional information previously agreed to by the donor for disclosure, according to the terms of their opt-in preferences.

To support the market research functionality, the system includes an opt-in workflow for donors during registration or via subsequent profile updates. Donors electing to participate in market research studies are prompted to complete additional data fields relevant to recruitment targeting, including consent to be contacted and compensation preferences. These data fields are stored in a secure, access-controlled portion of the donor profile and are included in search indexing for authorized users. Once a market research company initiates contact and the donor agrees to participate, the Donor X platform logs the engagement and may facilitate or track compensation events in accordance with the terms established during donor onboarding. Compensation may be rendered directly by the market research company or via integration with third-party fulfillment providers. This system architecture provides both donor privacy and operational transparency while enabling market research firms to efficiently recruit participants for surveys, product testing, focus groups, and other commercial research applications.

### EXAMPLES

Clause 1. A system for on-demand biospecimens comprising. a server comprising at least a processor, a data store, a transceiver, and code stored on the data store, which is executable by the processor, wherein the transceiver connects the server to a network; at least one donor computing device communication linked to the server via the network; and at least one specimen requestor computing device community linked to the server via the network, wherein the server coordinates timing and delivery of biospecimens provided by a set of donors, each utilizing a corresponding one of the at least one donor computing device, to a set of biospecimen requestors, each utilizing a corresponding one of the at least one specimen requestor computing device, wherein the biospecimens are provided by the specimen donors within three days of a request from the specimen requestor without any of the biospecimens being stored in a biobank or equivalent intermediary.
Clause 2. The system of clause 1, wherein the biospecimens as received by and requested from the specimen requestor are fresh and are never frozen between a period extracted from any of the specimen donors and received by the specimen requestor,
Clause 3. The system of clause 1, wherein each of the specimen donors are employees of the specimen requestor, said specimen donors having privacy and medical information protected in an FDA and HIPAA compliant manner, facilitated by the server.
Clause 4. The system of clause 1, wherein the specimens are collected and delivered in a same building, wherein specimen requestors receive the specimens within one hour of collection.
Clause 5. The system of clause 1, wherein the specimen donors are volunteers who are paid for each specimen provided, wherein payment amounts are displayed within request messages prior to the specimen donors agreeing to provide a related biospecimen.
Clause 6. A computer-implemented method executed by a biospecimen procurement platform comprising one or more processors and memory storing instructions that, when executed, cause a system to perform operations comprising; receiving, via an authenticated user interface, a structured electronic request from a scientist client device, the request including one or more of a biospecimen type, collection instructions, a subject demographic, or health criteria, and a desired delivery timeframe; generating, using a scheduling engine, a biospecimen job comprising one or more digitally indexed appointment slots, each slot including metadata specifying a collection location, collection time, biospecimen type, and packaging specification; executing a donor eligibility determination using a matching engine configured to apply rule-based and machine-learned models to filter a donor dataset based on demographic, geographic, and physiological safety thresholds, including longitudinal blood draw volume analysis; transmitting, via a push-notification messaging module, a set of donor-specific job invitations to mobile devices of eligible donors, the invitations including appointment metadata and system-generated response options, while concealing an identity of the scientist; receiving, from at least one donor device, a secure acceptance of an appointment slot, subject to confirmation that a digitally signed consent form of the donor is current, a travel radius constraint is met, and the donor is within a safe donation threshold; reserving the accepted appointment slot and generating an appointment confirmation object comprising a cryptographic job token, appointment timestamp, and location metadata, which is transmitted to the donor device; prior to the appointment time, initiating a timed notification workflow that includes calendar sync, secure rescheduling tokens, and SMS/email reminders to the donor; in response to a cancellation, reinitiating a donor matching and an invitation process via an automated feedback loop until the appointment is reassigned; receiving, from a laboratory information management system or integrated collection terminal, a structured data packet indicating collection confirmation, the packet including scanned container label identifiers, job ID, timestamp, and donor ID; updating, using a backend processing module, a scientist dashboard associated with the job to reflect the collection confirmation, de-identified donor metadata, and specimen tracking status; initiating a donor payment transaction via a digitally integrated payment processor upon the confirmation of collection, wherein the transaction is linked to a verified payment method of the donor stored in encrypted form; and logging a job lifecycle including one or more of a consent, an eligibility match, an appointment assignment, a specimen collection, or a payment confirmation in a queryable system database configured for regulatory auditing and data export.
Clause 7. The method of clause 6, wherein the donor eligibility determination comprises calculating a safe blood draw volume using historical donation data stored in a time-indexed database and automatically disqualifying donors exceeding regulatory limits.
Clause 8. The method of clause 6, wherein the cryptographic job token is a signed identifier generated using a private key stored on a hardware security module to verify integrity of the job metadata.
Clause 9. The method of clause 6, wherein the push-notification module delivers invitations via a third-party API supporting multi-channel delivery and records delivery status and interaction metrics for adaptive donor engagement.
Clause 10. The method of clause 6, wherein the collection confirmation includes scanning a machine-readable label affixed to each biospecimen container, the label encoding the donor ID, job ID, and timestamp using a QR or barcode format.
Clause 11. The method of clause 6, wherein the payment processor executes transactions via an API call to a third-party platform and logs payment status in real time.
Clause 12. The method of clause 6, further comprising assigning one or more backup donors to the job using a predictive model trained on historical no-show data and dynamically adjusting backup ratios based on region and sample type.
Clause 13, The method of clause 6, wherein the scientist dashboard includes an export function configured to generate a digitally signed report file comprising donor health metadata and chain-of-custody timestamps.
Clause 14. The method of clause 6, wherein the platform verifies user identity using a multi-factor authentication process that includes a biometric or knowledge-based identity check and an SMS-based one-time password.
Clause 15. The method of clause 6, wherein the system executes a consent verification engine to confirm that the donor's digitally signed informed consent form is valid, current, and linked to the requested biospecimen type.
Clause 16. A computer system with a platform for coordinating biospecimen collection, comprising: one or more processors and a non-transitory memory storing executable instructions configured to: receive a biospecimen request from a scientist interface; create a job comprising appointment slots with metadata including biospecimen types, collection location, and timing constraints; execute a donor matching engine to evaluate donor eligibility using profile data, longitudinal health history, and spatial-temporal constraints; generate and transmit dynamic electronic invitations to eligible donors while maintaining de-identification of the scientist; receive donor responses and reserve accepted appointment slots; synchronize calendar and notification systems for donor reminders and rescheduling; interface with laboratory systems to receive structured collection confirmations; update a scientist-facing dashboard with collection status and de-identified donor information; initiate secure donor payment using verified financial data; and store job lifecycle data in a secure, queryable database for reporting and compliance.
Clause 17. The system of clause 16, further comprising a container label generator configured to produce machine-readable specimen labels using specimen-specific and donor-specific encrypted metadata.
Clause 18. The system of clause 16, wherein the donor matching engine applies a trained neural network model to predict donor acceptance likelihood based on historical participation, location, and compensation preferences.
Clause 19. The system of clause 16, wherein the platform integrates with external laboratory systems using RESTful APIs and transmits real-time updates regarding appointment bookings, specimen readiness, and courier status.
Clause 20. The system of clause 16, wherein the scientist dashboard supports one or more of real-time job filtering, longitudinal tracking of participating donors, or tagging of donors to curated study lists for future recruitment.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The computer-readable instructions are provided to the processor 334 of a general purpose computer, special purpose computer, or other programmable data processing apparatus (e.g., the computing device 336) to produce a machine, such that the instructions, which execute via the processor 334 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 336), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages. including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions,

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions,

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for on-demand biospecimens comprising. a server comprising at least a processor, a data store, a transceiver, and code stored on the data store, which is executable by the processor, wherein the transceiver connects the server to a network;
at least one donor computing device communication linked to the server via the network; and
at least one specimen requestor computing device community linked to the server via the network, wherein the server coordinates timing and delivery of biospecimens provided by a set of donors, each utilizing a corresponding one of the at least one donor computing device, to a set of biospecimen requestors, each utilizing a corresponding one of the at least one specimen requestor computing device, wherein the biospecimens are provided by the specimen donors within three days of a request from the specimen requestor without any of the biospecimens being stored in a biobank or equivalent intermediary.

2. The system of claim 1, wherein the biospecimens as received by and requested from the specimen requestor are fresh and are never frozen between a period extracted from any of the specimen donors and received by the specimen requestor.

3. The system of claim 1, wherein each of the specimen donors are employees of the specimen requestor, said specimen donors having privacy and medical information protected in an FDA and HIPAA compliant manner, facilitated by the server.

4. The system of claim 1, wherein the specimens are collected and delivered in a same building, wherein specimen requestors receive the specimens within one hour of collection.

5. The system of claim 1, wherein the specimen donors are volunteers who are paid for each specimen provided, wherein payment amounts are displayed within request messages prior to the specimen donors agreeing to provide a related biospecimen.

6. A computer-implemented method executed by a biospecimen procurement platform comprising one or more processors and memory storing instructions that, when executed, cause a system to perform operations comprising:
receiving, via an authenticated user interface, a structured electronic request from a scientist client device, the request including one or more of a biospecimen type, collection instructions, a subject demographic, or health criteria, and a desired delivery timeframe;
generating, using a scheduling engine, a biospecimen job comprising one or more digitally indexed appointment slots, each slot including metadata specifying a collection location, collection time, biospecimen type, and packaging specification;
executing a donor eligibility determination using a matching engine configured to apply rule-based and machine-learned models to filter a donor dataset based on demographic, geographic, and physiological safety thresholds, including longitudinal blood draw volume analysis;
transmitting, via a push-notification messaging module, a set of donor-specific job invitations to mobile devices of eligible donors, the invitations including appointment metadata and system-generated response options, while concealing an identity of the scientist;
receiving, from at least one donor device, a secure acceptance of an appointment slot, subject to confirmation that a digitally signed consent form of the donor is current, a travel radius constraint is met, and the donor is within a safe donation threshold;
reserving the accepted appointment slot and generating an appointment confirmation object comprising a cryptographic job token, appointment timestamp, and location metadata, which is transmitted to the donor device;
prior to the appointment time, initiating a timed notification workflow that includes calendar sync, secure rescheduling tokens, and SMS/email reminders to the donor;
in response to a cancellation, reinitiating a donor matching and an invitation process via an automated feedback loop until the appointment is reassigned;
receiving, from a laboratory information management system or integrated collection terminal, a structured data packet indicating collection confirmation, the packet including scanned container label identifiers, job ID, timestamp, and donor ID;
updating, using a backend processing module, a scientist dashboard associated with the job to reflect the collection confirmation, de-identified donor metadata, and specimen tracking status;
initiating a donor payment transaction via a digitally integrated payment processor upon the confirmation of collection, wherein the transaction is linked to a verified payment method of the donor stored in encrypted form; and
logging a job lifecycle including one or more of a consent, an eligibility match, an appointment assignment, a specimen collection, or a payment confirmation in a queryable system database configured for regulatory auditing and data export.

7. The method of claim 6, wherein the donor eligibility determination comprises calculating a safe blood draw volume using historical donation data stored in a time-indexed database and automatically disqualifying donors exceeding regulatory limits.

8. The method of claim 6, wherein the cryptographic job token is a signed identifier generated using a private key stored on a hardware security module to verify integrity of the job metadata.

9. The method of claim 6, wherein the push-notification module delivers invitations via a third-party API supporting multi-channel delivery and records delivery status and interaction metrics for adaptive donor engagement.

10. The method of claim 6, wherein the collection confirmation includes scanning a machine-readable label affixed to each biospecimen container, the label encoding the donor ID, job ID, and timestamp using a QR or barcode format.

11. The method of claim 6, wherein the payment processor executes transactions via an API call to a third-party platform and logs payment status in real time.

12. The method of claim 6, further comprising assigning one or more backup donors to the job using a predictive model trained on historical no-show data and dynamically adjusting backup ratios based on region and sample type.

13. The method of claim 6, wherein the scientist dashboard includes an export function configured to generate a digitally signed report file comprising donor health metadata and chain-of-custody timestamps.

14. The method of claim 6, wherein the platform verifies user identity using a multi-factor authentication process that includes a biometric or knowledge-based identity check and an SMS-based one-time password.

15. The method of claim 6, wherein the system executes a consent verification engine to confirm that the donor's digitally signed informed consent form is valid, current, and linked to the requested biospecimen type.

16. A computer system with a platform for coordinating biospecimen collection, comprising:
one or more processors and a non-transitory memory storing executable instructions configured to:
receive a biospecimen request from a scientist interface;
create a job comprising appointment slots with metadata including biospecimen types, collection location, and timing constraints;
execute a donor matching engine to evaluate donor eligibility using profile data, longitudinal health history, and spatial-temporal constraints;
generate and transmit dynamic electronic invitations to eligible donors while maintaining de-identification of the scientist;
receive donor responses and reserve accepted appointment slots;
synchronize calendar and notification systems for donor reminders and rescheduling;
interface with laboratory systems to receive structured collection confirmations;
update a scientist-facing dashboard with collection status and de-identified donor information;
initiate secure donor payment using verified financial data;
and store job lifecycle data in a secure, queryable database for reporting and compliance.

17. The system of claim 16, further comprising a container label generator configured to produce machine-readable specimen labels using specimen-specific and donor-specific encrypted metadata.

18. The system of claim 16, wherein the donor matching engine applies a trained neural network model to predict donor acceptance likelihood based on historical participation, location, and compensation preferences.

19. The system of claim 16, wherein the platform integrates with external laboratory systems using RESTful APIs and transmits real-time updates regarding appointment bookings, specimen readiness, and courier status.

20. The system of claim 16, wherein the scientist dashboard supports one or more of real-time job filtering, longitudinal tracking of participating donors, or tagging of donors to curated study lists for future recruitment.
